(19) United States
(12) Patent Application Publication
(10) Pub. No.: US 2004/0146896 A1
(43) Pub. Date: Jul. 29, 2004

(54) Methods and compositions for diagnosing and treating companion animal cancer

(76) Inventors: Sing Rong, Kalamazoo, MI (US); Jenny Hong Cai, Niantic, CT (US)

Correspondence Address: SCULLY SCOTT MURPHY & PRESSER, PC, 400 GARDEN CITY PLAZA, GARDEN CITY, NY, 11530

(21) Appl. No.: 10/696,935

(22) Filed: Oct. 30, 2003

Related U.S. Application Data
(60) Provisional application No. 60/422,342, filed on Oct. 30, 2002.

(51) Int. Cl. C12Q 1/68; G01N 33/574; C07H 21/04; C07K 14/705

(52) U.S. Cl. 435/6; 435/7.23; 435/69.1; 435/320.1; 435/325; 530/350; 536/23.5

(57) ABSTRACT

The present invention relates to polynucleotides associated with apoptosis-related disorders. The present invention also relates to canine and feline TRAIL genes, novel genes associated with apoptosis-related disorders, such as cancer. The invention encompasses TRAIL nucleic acids, recombinant DNA molecules, cloned genes or degenerate variants thereof, TRAIL gene products and antibodies directed against such gene products, cloning vectors containing mammalian TRAIL gene molecules, and hosts that have been genetically engineered to express such molecules. The invention further relates to methods for the identification of compounds that modulate the expression of TRAIL genes and gene products and to the use of such compounds as therapeutic agents in the treatment of apoptosis-related disorders, e.g., cancer. The invention also relates to methods for the diagnostic evaluation, genetic testing and prognosis of apoptosis-related disorders, e.g., cancer, and to methods and compositions for the treatment these disorders.

### CROSS REFERENCE TO RELATED APPLICATION

This application claims priority from U.S. Provisional Application No. 60/422,342, filed on Oct. 30, 2002.

### FIELD OF THE INVENTION

The present invention relates to polynucleotide sequences which are shown herein to be associated with the induction of apoptosis of cancer cells. More specifically, the present invention relates to novel polynucleotide sequences which encode the cancer cell apoptosis inducer, TRAIL (TNF-related apoptosis-inducing ligand). The invention encompasses nucleic acids which encode TRAIL protein, recombinant DNA molecules, cloned genes and degenerate variants thereof, vectors containing such TRAIL-encoding nucleic acids, and hosts that have been genetically engineered to express and/or contain such molecules. The invention further relates to TRAIL gene products and antibodies directed against such gene products. The invention further relates to methods of identifying compounds that modulate the expression, synthesis and activity of such TRAIL-encoding nucleic acids, and to methods of using compounds such as those identified herein as therapeutic agents in the treatment of apoptosis-related disorders, including, but not limited to, cancer as well as neurodegenerative disease, lupus erythematosus, rheumatoid arthritis, multiple sclerosis, for example. The invention also relates to methods for the diagnostic evaluation, genetic testing and prognosis of apoptosis-related disorders, including, but not limited to, cancer as well as neurodegenerative disease, lupus erythematosus, rheumatoid arthritis, multiple sclerosis, for example.

### BACKGROUND OF THE INVENTION

The apoptosis process is crucial to various biological processes including embryo development and organism homeostasis. Apoptosis plays very important physiological roles such as elimination of virus-infected cells, deletion of activated lymphocytes at the end of immune responses and removal of abnormal cells in the body (Jacobson et al, 1997, Cell 88: 347-354). Through evolution, biological systems, especially mammals, developed apoptosis-signaling mechanisms that actively instruct certain cells to die. Apoptosis can be induced by a variety of stimuli that include anti-cancer drugs, deprivation of growth factors as well as death factors (Nagata, 1997, Cell 88:355-365). The mechanisms relating to death factors involve the interactions between cell-surface death receptors and death ligands. Three of these ligands, TNF (tumor necrosis factor), Fas/CD95L, and LT-alpha have received particular attention because they can induce apoptosis of tumor cells. However, the potential use of these three proteins is limited because of their acute toxic effect on normal tissues in vivo.

TRAIL is a type II transmembrane protein which was initially identified as a homolog of its extracellular domain with TNF, CD95L and LT-alpha (Wiley et al, 1995, Immunity 3:673-682). The C-terminal extracellular region of TRAIL is conserved among the family members, has a “jelly-roll” structure and forms a trimer (Ashkenazi & Dixit, 1998, Science 281:1305-1308). TRAIL protein has been reported to induce apoptosis in various tumor cell lines but not in nontransformed, normal cells (Walczak et al, 1999, Nature Med. 5:157-163; Wiley et al, 1995, Immunity 3:673-682: Pitti et al, 1996, J. Biol. Chem. 271:12687-12690). In addition, preclinical studies in mice and nonhuman primates have shown that administration of TRAIL can induce apoptosis in human tumors, but that no cytotoxicity in normal organs or tissues is found (Walczak et al, 1999, Nature Med. 5:157-163; Ashkenzi et al, 1999, J. Clin. Invest. 104:155-162).

In 2000, a polyhistidine-tagged recombinant human TRAIL (residues 114-281) was found to induce apoptosis in vitro in isolated human heptocytes, but not in non-human hepatocytes (Jo et al. 2000, Nature Med. 6: 564-567). This has raised concerns that using TRAIL in cancer therapy might cause liver toxicity in vivo (Nagata, 2000, Nature Med. 6 (5): 502-503). Recently, however, differential hepatocyte toxicity and other biochemical properties of recombinant TRAIL versions were reported (Lawrence et al, 2001, Nature Med. 7 (4): 384-385). The native form of soluble TRAIL protein showed no effect while His-tagged TRAIL induced substantial apoptosis in the assays.

Cancer is not only devastating to humans, but also the most common cause of natural death in dogs (Bronson, 1982, Am J Vet Res, 43(11) 2057-9). Dogs develop tumors twice as frequently as humans do, and it has been reported that 45-50% of dogs that live to 10 years or older succumb to cancer, regardless of age; and that 23% of dogs that present for necropsy died of cancer (Bronson, 1982, Am J Vet Res, 43(11) 2057-9). Surgical removal of the tumor is the most common treatment, but the prognosis for dogs having invasive/metastatic tumor is very poor, with median survival time ranging from weeks to months. Other treatments, such as radiation therapy and chemotherapy, have only very limited success (Bostock, 1986, Br Vet J 142(6):506-15; Bostock, 1986, Br Vet J 142(1):1-19; MacEwen, 1990, Cancer Metastasis Rev 9(2): 125-36). Thus, more effective treatments for angiogenic diseases, such as, for example, canine cancers, are necessary.

### SUMMARY OF THE INVENTION

The present invention encompasses novel nucleotide sequences that are associated with apoptosis related disorders, e.g., cancer. Specifically, the present invention is directed to nucleotide sequences that encode TRAIL. One aspect of the present invention is directed to TRAIL-encoding nucleic acids, recombinant DNA molecules, cloned genes or degenerate variants thereof. Another aspect of the present invention is directed to vectors, including expression vectors, containing TRAIL-encoding nucleic acid molecules, and hosts that have been genetically engineered to express and/or contain such TRAIL gene products.

The present invention further encompasses novel TRAIL gene products and antibodies directed against such gene products, or variants or fragments thereof.

In one aspect, the present invention is directed to methods for modulation of TRAIL-mediated processes and for the treatment of disorders involving apoptosis, such as cancer, including the amelioration or prevention of at least one symptom of the disorders, wherein such methods comprise administering a compound which modulates the expression of a TRAIL gene and/or the synthesis or activity of a TRAIL gene product. A particular aspect of the present invention is directed to methods for the use of a TRAIL gene product or fragment, analog, or mimetic thereof, or an antibody or antibody fragment directed against a TRAIL gene product, to treat or ameliorate a symptom of such disorders.

In another aspect, the present invention is directed to methods for modulation of TRAIL-mediated processes and for the treatment of disorders involving abnormal apoptosis of cells, including the amelioration or prevention of at least one symptom of the disorders, wherein such methods comprise administering a compound which modulates the expression of a TRAIL gene and/or the synthesis or activity of a TRAIL gene product. The term “TRAIL-mediated process” as used in the present invention, includes processes dependent on and/or responsive to, either directly or indirectly, the level of expression, gene product synthesis and/or gene product activity of TRAIL genes.

A particular aspect of the present invention is directed to methods for the use of a novel TRAIL gene product or fragment, analog, or mimetic thereof, or an antibody or antibody fragment directed against a TRAIL gene product, to treat or ameliorate a symptom of such disorders.

In still another aspect, the present invention is directed to methods for blocking interactions between TRAIL and its respective receptors with analogs that act as receptor antagonists. These antagonists can promote apoptosis. Such effects are desirable in situations including, but not limited to, inadequate apoptosis of activated T cells and autoimmune diseases.

In a particular aspect, methods of the present invention can comprise modulating the level of expression or the activity of a TRAIL gene product in a cell such that the TRAIL-mediated process or the disorder is treated, e.g., a symptom is ameliorated. In another particular aspect, such methods can comprise supplying a nucleic acid molecule encoding a TRAIL gene product to increase the level, expression or activity of the TRAIL gene product within the cell such that the TRAIL-mediated process or the disorder is treated, e.g., a symptom is ameliorated. The nucleic acid molecule encoding the TRAIL gene product can encode a mutant TRAIL gene product with increased activity or expression levels.

In yet another aspect, the present invention is directed to methods for modulation of TRAIL-mediated processes or the treatment of TRAIL-related disorders, such as cancer, including, but not limited to, disorders resulting from TRAIL gene mutations, and/or an abnormal levels of TRAIL expression or activity and disorders involving one or more TRAIL genes or gene products, wherein treatment includes the amelioration or prevention of at least one symptom of such disorders. The term “TRAIL-related disorder” as used in the present invention, refers to disorders involving a TRAIL gene or gene product, or an aberrant level of TRAIL gene expression, gene product synthesis and/or gene product activity, respectively, relative to levels found in normal, unaffected, unimpaired individuals, levels found in clinically normal individuals, and/or levels found in a population whose levels represent baseline, average TRAIL levels. In a particular aspect, such methods can comprise supplying a mammal in need of treatment with a nucleic acid molecule encoding an unimpaired TRAIL gene product such that the unimpaired TRAIL gene product is expressed and the disorder is treated, e.g., a symptom is ameliorated. By “unimpaired TRAIL gene product” is meant the unchanged or native form of TRAIL gene product. In another particular aspect, such methods can comprise supplying a mammal in need of treatment with a cell comprising a nucleic acid molecule that encodes an unimpaired TRAIL gene product such that the cell expresses the unimpaired TRAIL gene product and the disorder is treated, e.g., a symptom is ameliorated. In yet another particular aspect, such methods comprise supplying a mammal in need of treatment with a modulatory compound, such as, for example, a small molecule, peptide or antibody that is capable of modulating the activity of a TRAIL gene or gene product.

In addition, the present invention is directed to methods that utilize TRAIL gene sequences and/or TRAIL gene product sequences for diagnostic evaluation, genetic testing and/or prognosis of angiogenesis-related disorders, such as cancer. For example, the invention relates to methods for diagnosing apoptosis-related disorders, e.g., cancer, wherein such methods can comprise measuring TRAIL gene expression in a patient sample, or detecting a TRAIL mutation that correlates with the presence or development of such a disorder, in the genome of a mammal suspected of exhibiting such a disorder.

The present invention is also directed to utilizing the TRAIL gene sequences and/or gene products as markers for mapping of the human chromosome.

The invention is further directed to methods for identifying compounds capable of modulating the expression of an TRAIL gene and/or the synthesis or activity of an TRAIL gene product, wherein such methods comprise contacting a compound with a cell that expresses such a TRAIL gene, measuring the levels of TRAIL gene expression, gene product expression or gene product activity, and comparing such levels to the levels of TRAIL gene expression, gene product, or gene product activity produced by the cell in the absence of such compound, such that if the level obtained in the presence of the compound differs from that obtained in its absence, a compound capable of modulating the expression of the TRAIL gene and/or the synthesis or activity of the TRAIL gene product is identified.

Definitions

As used herein, the following terms shall have the abbreviations indicated.

BAC: bacterial artificial chromosome

bp: base pair(s)

dbEST: expressed sequence tag data base (National Center for Biotechnology Information)

EST: expressed sequence tag

Oligos: deoxyoligonucleotides

RT-PCR: reverse transcriptase PCR

SNP: single nucleotide polymorphism

SSCP: single-stranded conformational polymorphism

YAC: yeast artificial chromosome

### BRIEF DESCRIPTION OF THE FIGURES

FIG. 1: TRAIL gene expression in canine and feline cell lines. Total RNA was isolated from cell lines CRL-6130 (Fc28.Lu, feline lung), CRL-6252 (ECF50.HT, canine heart), CRL-6569 ((Fc2.Lu, feline lung). Two pairs (SEQ ID NO: 1 with SEQ ID NO: 2 for Pair 1; SEQ ID NO: I with SEQ ID NO: 3 for Pair 2) of primers were used to amplify internal regions of canine and feline TRAIL. 1 ug of total RNA was added to the RT-PCR beads (Amersham Pharmacia, cat no. 27-9267-01, Illinois) together with 1 ug of pd(N)₆ as the first strand primer. The PCR reaction was carried out after inactivating the reverse transcriptase and completely denaturing the template. The PCR products were analyzed by electrophoresis on 1.2% of agarose gel.

FIG. 2: The nucleotide sequence of canine TRAIL gene (SEQ ID NO: 20). The sequence in bold indicates the sequences for open reading frame. The sequences underlined indicate the start codon and termination codon.

FIG. 3: Canine TRAIL protein. The amino acid sequence of canine TRAIL translated from the sequence of FIG. 2 (SEQ ID NO: 21).

FIG. 4: The nucleotide sequence of feline TRAIL gene (SEQ ID NO: 22). The sequence in bold indicates the sequences for open reading frame. The sequences underlined indicate the start codon and termination codon.

FIG. 5: Feline TRAIL protein (SEQ ID NO: 23).

FIGS. 6A-6D: Alignment of all known (human, mouse, canine and feline) amino acid sequences of TRAIL. Bold-faced residues indicate difference from consensus.

FIGS. 7A-7B: Alignment of all known (human, mouse, canine and feline) amino acid sequences of soluble TRAIL. Bold-faced residues indicate difference from consensus.

FIGS. 8A-8B: Western immunoblot analysis of TRAIL proteins expressed in mammalian cells. Human 293 cells were transfected with plasmids encoding canine or feline TRAIL using CalPhos Mammalian Transfection Kit (CLONTECH Laboratories, Inc., Palo Alto, Calif.). 2 days post-transfection, both culture supernatant and cell lysate were prepared. The proteins were separated by 4-20% SDS-PAGE and transferred to PVDF membrane (NOVEX, San Diego, Calif.). For immunoblot analysis, either HA antibody (FIG. 8A) or anti-human TRAIL antisera (R & D Systems, FIG. 8B) were used as probes. The bound antibody was detected using phosphatase substrate BCIP/NBT (KPL, Gaithersburg, Md.). Marker, molecular weight marker; caTRAIL, canine TRAIL; feTRAIL, feline TRAIL; pGL2, a plasmid used as transfection control.

FIGS. 9A-9C: Apoptosis assays for mammalian expressed TRAIL. U937 human tumor cells were grown in the presence or absence of TRAIL proteins produced from conditioned media of transfected 293 cells. FIG. 9A shows the growth inhibition by mammalian expressed TRAIL in MTT growth inhibition assay. FIG. 9B shows the apoptosis inducing activity in Cell Death ELISA assay. FIG. 9C shows the apoptosis inducing activity in Annexin V FACS apoptosis analysis. Stau, staurosporin (0.4 uM), a potent apoptosis inducer; huTRAIL, human TRAIL (Upstate Biotechnology, 100 ng/ml); caTRAIL, canine TRAIL; feTRAIL, feline TRAIL; vector, a plasmid used as transfection control.

FIGS. 10A-10E: Expression and solubility of TRAIL protein expressed from pBAD-thio promoter in bacteria. E. coli TOP 10 cells expressing pBAD-Thio-TRAIL plasmids under various arabinose induction conditions (a, 0.2% arobinose; b, 0.02%; c, 0.002%; and d, no arobinose) were collected and sonicated to obtain soluble and insoluble fractions of the lysates. Equal volumes of each fraction were loaded onto 10% Bis-Tris gel (Invitrogen, NuPAGE 1.0 mm×15 well, NP0303) and stained with 0.25% (w/v) Brillant Blue R. FIGS. 10A-10B, TRAIL expression under various inducible conditions. FIGS. 10C-10E, TRAIL protein solubility (P, pellet; S, soluble fractions) under various inducible conditions. Hu-T, human TRAIL with V5-His tag; Hu, human TRAIL without tag; Ca-T, canine TRAIL with tag; Ca, canine TRAIL without tag; Fe-T, feline TRAIL with tag; Fe, feline TRAIL without tag.

FIGS. 11A-11B: His-Band Quick 900 cartridges column fractions of human and canine TRAIL-Thio. Bacterial cell lysates containing human and canine TRAIL-Thio proteins were loaded onto His-band Quick 900 cartridges (Novagen). Eight fractions (Elute, 0.5 ml/fraction) were collected and loaded onto 10% Bis-Tris gel (Invitrogen, NuPAGE 1.0 mm×15 well, NP0303) and stained with 0.25% (w/v) Brillant Blue R. F 1, flowthrough of lysate passed column once; F2, flowthrough of lysate passed column twice; W1, column wash with binding buffer; W2, column wash with washing buffer.

FIG. 12: Apoptosis assays for bacteria expressed TRAIL-Thio proteins. U937 human tumor cells were grown in the presence or absence of human and canine TRAIL-Thio proteins expressed and purified from bacterial cells. Apoptosis inducing activity was measured in Annexin V FACS analysis. Staurosporin (0.4 uM), Fas Ligand (100 ng/ml, Upstate Biotechnlogy) and commercial human TRAIL (100 ng/ml, Upstate Biotechnology) were used as positive controls. Two human TRAIL-Thio preps (huTRAIL E3 and E4) and two canine TRAIL-Thio preps (caTRAIL E3 and E2+E4) were used in the assay.

FIGS. 13A-13B: Expression of TRAIL protein expressed from T7 promoter in bacteria. E. coli BL21 cells expressing pT7-TRAIL plasmids in the presence (+) or absence (−) of IPTG induction were collected and lysed into the SDS sample buffer, followed by loading onto 10% Bis-Tris gel (Invitrogen, NuPAGE 1.0 mm×15 well, NP0303) and transferred to Immobilon P (Millipore) for Western analysis. FIG. 13A, anti-V5 antibody (Invitrogen) was used as probe. FIG. 13B, anti-TRAIL protein (AF375, R & D Systems) was used as probe. T, TRAIL proteins with V5-His tag; N, TRAIL proteins without tag; Hu, human TRAIL; Ca, canine TRAIL; Fe, feline TRAIL; CAT gene, chloramphenicol acetyltransferase gene, used as a positive control. TRAIL-H, TRAIL protein with V5-His tag; TRAIL, TRAIL protein without tag.

FIGS. 14A-14B: Solubility of TRAIL protein expressed from T7 promoter in bacteria. E. coli BL21 cells expressing pT7-huTRAIL plasmids were lysed and centrifuged three times, followed by loading onto an anion exchange Q Fast-Flow column and a cation exchange S-sepharose column. Each samples were electrophoresed on 10% Bis-Tris gel (Invitrogen, NuPAGE 1.0 mm×15 well, NP0303) and stained with 0.25% (w/v) Brillant Blue R (FIG. 14A). Or the gel was transferred to Immobilon P (Millipore) for Western analysis using anti-TRAIL protein (AF375, R & D Systems) as probe (FIG. 14B). Lane 1, lysate; 2, elute of Q column; 3, flowthrough of Q column; 4, sample after dialysis; 5, supernatant from after the second centrifugation; 6, pellet from the first centrifugation; 7, pellet from the second centrifugation; 8, pellet from the third centrifugation; 9, flowthrough of SP column; 10, wash of SP column; 11, elute of SP column.

FIGS. 15A-15C: Expression of TRAIL protein expressed from pBAD promoter in bacteria. E. coli TOP 10 cells expressing canine and feline pBAD-TRAIL plasmids in the presence (+, 0.02%) or absence (−) arabinose induction were lysed into the SDS sample buffer, followed by loading onto 10% Bis-Tris gel (Invitrogen, NuPAGE 1.0 mm×15 well, NP0303) and stained with 0.25% (w/v) Brillant Blue R (FIG. 15A). Or the gel was transferred to Immobilon P (Millipore) for Western analysis (FIGS. 15B & 15C). Panel B, anti-TRAIL protein (AF375, R & D Systems) antibody was used as probe. Panel C, anti-V5 (Invitrogen) was used as probe. Two clones of each plasmid constructs were tested. Ca, canine TRAIL; Fe, feline TRAIL; CaH, canine TRAIL with V5-His tag; FeH, feline TRAIL with V5-His tag. TRAIL-H, TRAIL protein with V5-His tag; TRAIL, TRAIL protein without any tag.

FIGS. 16A-16B: Solubility of TRAIL protein expressed from pBAD promoter in bacteria. E. coli TOP 10 cells expressing both canine and feline pBAD-TRAIL plasmids under 0.02% arabinose induction were collected and sonicated to obtain soluble (S) and insoluble (P) fractions of the lysates. Two different growth temperatures (30° C. and 37° C.) were also used. Equal volumes of each fraction were loaded onto 10% Bis-Tris gel (Invitrogen, NuPAGE 1.0 mm×15 well, NP0303) and stained with 0.25% (w/v) Brillant Blue R (FIG. 16A). Or the gel was transferred to Immobilon P (Millipore) for Western analysis with anti-TRAIL antibody (AF375, R & D Systems, FIG. 16B). Ca, canine TRAIL; Fe, feline TRAIL; CaH, canine TRAIL with V5-His tag; FeH, feline TRAIL with V5-His tag. TRAIL-H, TRAIL protein with V5-His tag; TRAIL, TRAIL protein without any tag.

FIGS. 17A-17H: His-Band Quick 900 cartridges column fractions of canine and feline TRAIL. Bacterial cell lysates containing canine (FIGS. 17A & 17B) and feline TRAIL (FIGS. 17C & 17D) proteins were loaded onto His-band Quick 900 cartridges (Novagen). Eight to ten fractions (Elute, 0.5 ml/fraction) were collected and 6 ul was loaded onto 10% Bis-Tris gel (Invitrogen, NuPAGE 1.0 mm×15 well, NP0303) and stained with SilverXpress staining kit (FIGS. 17A-17D, Invitrogen). Or the gel was transferred to Immobilon P (Millipore) for Western immunoblot analysis with anti-TRAIL antibody (FIGS. 17E-17H, AF375, R & D Systems, FIG. 17F). M, molecular weight marker; F, flowthrough of lysate; W1, wash of column with binding buffer; W2, wash of column with wash buffer. TRAIL-H, TRAIL protein with V5-His tag; TRAIL, TRAIL protein without any tag.

FIGS. 18A-18C: Apoptosis assays for bacteria expressed TRAIL proteins. U937 human tumor cells were grown in the presence or absence of TRAIL proteins produced from bacterial cells. Apoptosis inducing activity was measured in MTT growth inhibition assay (FIG. 18A); Cell Death ELISA apoptosis assay (FIG. 18B); Annexin V FACS apoptosis assay (FIG. 18C). PBS, phosphate-buffered saline; Staurosporin (0.4 uM) and commercial human TRAIL (huTRAIL, 100 ng/ml, Upstate Biotechnology) were used as positive controls. Also used as positive controls for the assays are the mammalian expressed TRAIL proteins supernatant (caTRAIL, feTRAIL and vector plasmid as control). Bacterial expressed samples are S5-S9. S5, bacterial column fractions which do not contain TRAIL, serve as negative control; S6, purified feline TRAIL from His-Band Quick 900 cartridges column. S7, purified feline TRAIL with V5-His tag. S8, semi-purified canine TRAIL, wash from the column. S9, semi-purified feline TRAIL, wash from the column.

FIGS. 19A-19B:Apoptosis assays of mammalian expressed TRAIL proteins for various human cancer cell lines. Human tumor cells (HELA, PT-3, HT-29, SW480 and U937) were grown in the presence or absence of mammalian expressed TRAIL protein supernatants. Apoptosis inducing activity was measured in MTT growth inhibition assay (FIG. 19A) and Cell Death ELISA apoptosis assay (FIG. 19B). Staurosporin (0.4 uM) and commercial human TRAIL (100 ng/ml, Upstate Biotechnology) were used as positive controls. The supernatant from vector plasmid transfected cells was used as a negative control. Data shown are the mean of 8.

FIGS. 20A-20B: Apoptosis assays of mammalian expressed TRAIL proteins for various canine cancer cell lines. Canine cells (D22, D17, CF21.T, CF11.T, MDCK, DH82, 0309 and 030E, human tumor cells U937 were used as control) were grown in the presence or absence of mammalian expressed TRAIL protein supernatants. Apoptosis inducing activity was measured in MTT growth inhibition assay (FIG. 20A) and Cell Death ELISA assay (FIG. 20B); Staurosporin (0.4 uM) and commercial human TRAIL (Upstate Biotechnology, 100 ng/ml for Cell Death ELISA assay only) were used as positive controls. The supernatant from vector transfected cells was used as a negative control. Data shown are the means of triplicates for MTT assay and duplicates for Cell Death ELISA assay.

FIGS. 21A-21B: Apoptosis assays of mammalian expressed TRAIL proteins for dog hepatocyte cells. Normal dog hepatocytes were grown in the presence or absence of mammalian expressed canine and feline TRAIL protein supernatants (caTRAIL and feTRAIL). Both Fas ligand (100 ng/ml, Upstate Biotechnology) as well as commercial human TRAIL (100 ng/ml, Upstate Biotechnology) were used as controls for the assay. Apoptosis inducing activity was measured in MTT growth inhibition assay (FIG. 21A); Cell Death ELISA apoptosis assay (FIG. 21B).

### BRIEF DESCRIPTION OF THE TABLE

Table 1. Cross-reactivity of canine and feline TRAIL proteins with various anti-human TRAIL antibodies.

### DETAILED DESCRIPTION OF THE INVENTION

Compositions and methods relating to nucleic acid sequences associated with disorders involving apoptosis are described herein. Novel genes which are associated with apoptosis-related disorders have been identified. Such genes encode TRAIL. In particular, described below are TRAIL nucleic acid molecules, as well as vectors comprising these molecules, host cells engineered to contain and/or express such molecules, TRAIL gene products, and antibodies that specifically recognize such gene products. Also described are various uses of these nucleic acids, polypeptides, and antibodies, as well as methods for their detection. For example, methods for the use of these molecules for modulation of apoptosis-related processes and for treatment of angiogenesis-related disorders, such as cancer, are described. Screening assays for compounds that interact with a TRAIL gene or gene product, or modulate TRAIL gene or gene product activity are also described below. Methods of treatment of an apoptosis-related disorder using the compositions of the invention and compositions identified by the methods of the invention are further described. Finally, pharmaceutical compositions for use with the compositions of the invention are described.

TRAIL nucleic acid molecules are described in this section. Unless otherwise stated, the term “TRAIL nucleic acid” refers collectively to the sequences described herein.

The TRAIL nucleic acid molecules of the invention include:

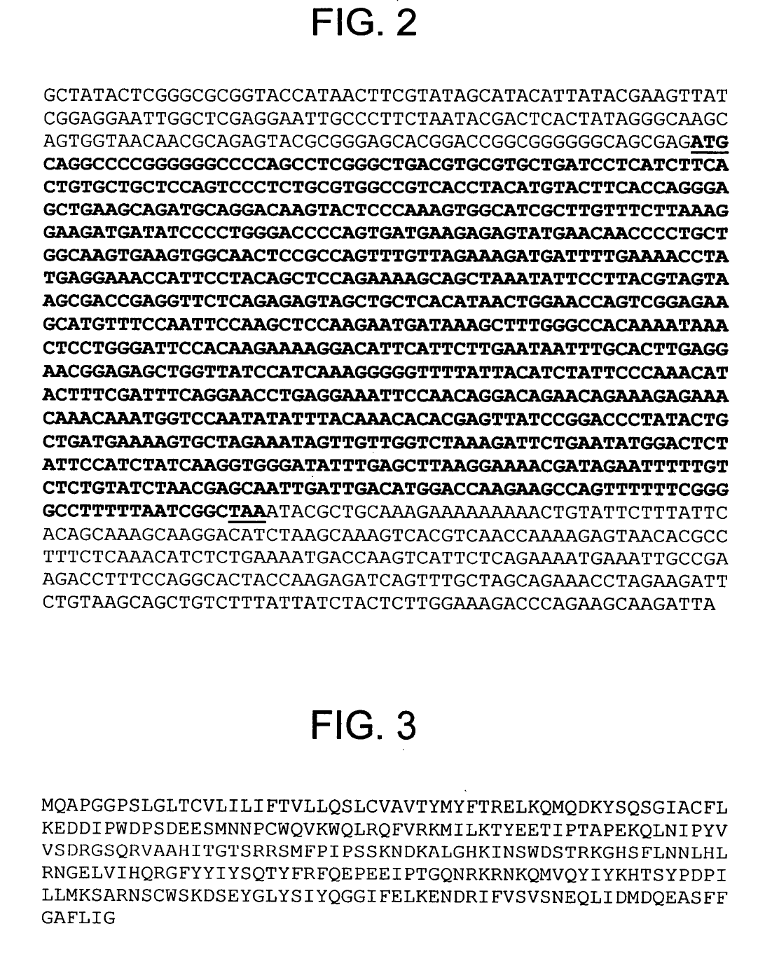

(a) a nucleic acid molecule containing the DNA sequence of canine TRAIL (FIG. 2, SEQ ID NO: 20) and fragments thereof;

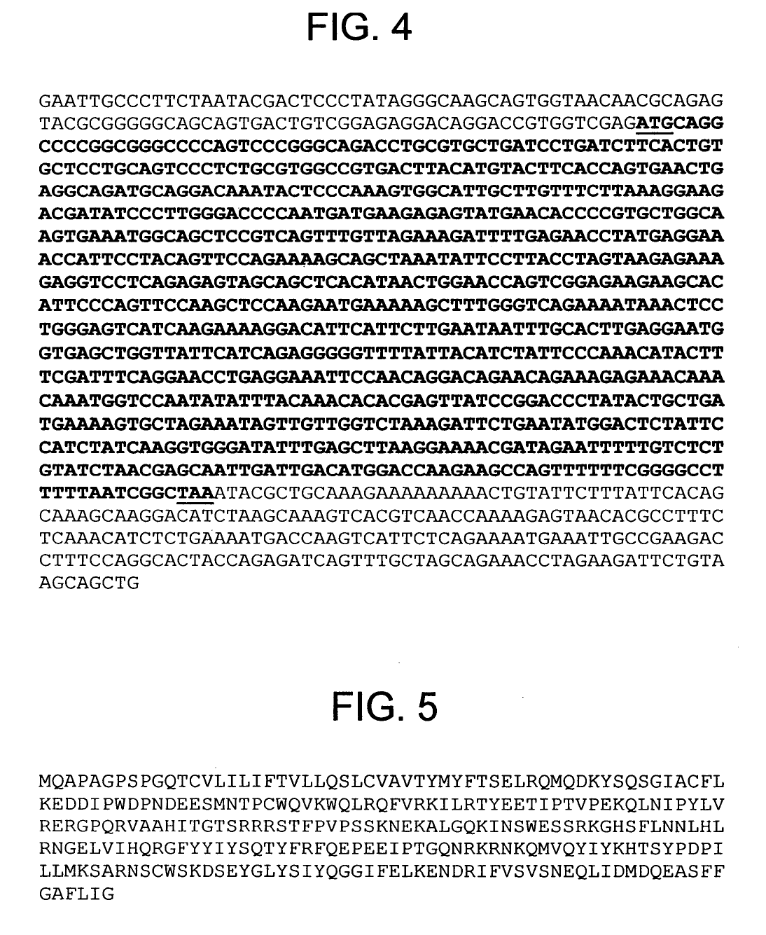

(b) a nucleic acid molecule containing the DNA sequence of feline TRAIL (FIG. 4, SEQ ID NO: 22) and fragments thereof;

(c) a nucleic acid molecule comprising a canine TRAIL nucleic acid sequence (e.g., the nucleic acid sequences depicted in FIG. 2 (SEQ ID NO: 20)) or a fragment thereof;

(d) a nucleic acid molecule comprising a feline TRAIL nucleic acid sequence (e.g., the nucleic acid sequences depicted in FIG. 4 (SEQ ID NO: 22) or a fragment thereof;

(e) a nucleic acid molecule that encodes a canine TRAIL gene product;

(f) a nucleic acid molecule that encodes a feline TRAIL gene product;

(g) a nucleic acid molecule that comprises at least one exon of a canine TRAIL gene;

(h) a nucleic acid molecule that comprises at least one exon of a feline TRAIL gene;

(i) a nucleic acid molecule that comprises TRAIL gene sequences of upstream untranslated regions, intronic regions, and/or downstream untranslated regions, or fragments thereof, of the canine TRAIL nucleotide sequences in (c) above;

(j) a nucleic acid molecule that comprises TRAIL gene sequences of upstream untranslated regions, intronic regions, and/or downstream untranslated regions, or fragments thereof, of the feline TRAIL nucleotide sequences in (d) above;

(k) a nucleic acid molecule comprising the novel TRAIL sequences disclosed herein that encodes mutants of the canine TRAIL gene products in which all or a part of one or more of the domains is deleted or altered, and fragments thereof;

(l) a nucleic acid molecule comprising the novel TRAIL sequences disclosed herein that encodes mutants of the feline TRAIL gene products in which all or a part of one or more of the domains is deleted or altered, and fragments thereof;

(m) nucleic acid molecules that encode fusion proteins comprising a canine TRAIL gene product, or a fragment thereof, fused to a heterologous polypeptide;

(n) nucleic acid molecules that encode fusion proteins comprising a feline TRAIL gene product, or a fragment thereof, fused to a heterologous polypeptide;

(o) nucleic acid molecules within the canine and feline TRAIL genes described in c) and d), above (e.g., primers), or within chromosomal nucleotide sequences flanking the TRAIL gene, which can be utilized as part of the methods of the invention for identifying and diagnosing individuals at risk for, or exhibiting an apoptosis-related disorder, such as cancer, or can be used for mapping human chromosomes; and;

(p) nucleic acid molecules within the canine and feline TRAIL genes described in c) and d), above, or within chromosomal nucleotide sequences flanking the canine and feline TRAIL genes, which correlate with an apoptosis-related disorder, such as cancer.

The TRAIL nucleotide sequences of the invention further include nucleotide sequences corresponding to the nucleotide sequences of (a)-(p) above wherein one or more of the exons, or fragments thereof, have been deleted.

The TRAIL nucleotide sequences of the invention also include nucleotide sequences greater than 20, 30, 40, 50, 60, 70, 80, 90, 100, or more base pairs long that have at least 75%, 80%, 85%, 90%, 95%, 98%, or more nucleotide sequence identity to the TRAIL nucleotide sequences of (a)-(p) above. However, it is understood that the nucleic acid molecules of the invention do not include nucleic acid molecules that consist solely of the nucleotide sequences from dbEST.

The TRAIL nucleotide sequences of the invention further include nucleotide sequences that encode polypeptides having at least 75%, 80%, 85%, 90%, 95%, 98%, or higher amino acid sequence identity to the polypeptides encoded by the TRAIL nucleotide sequences of (a)-(p) above.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of overlapping positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The TRAIL nucleotide sequences of the invention further include: (a) any nucleotide sequence that hybridizes to a TRAIL nucleic acid molecule of the invention under stringent conditions, e.g., hybridization to filter-bound DNA in 6× sodium chloride/sodium citrate (SSC) at about 45° C. followed by one or more washes in 0.2×SSC/0.1% SDS at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the TRAIL nucleic acid molecule that hybridizes under conditions described under (a) and (b), above, is one that comprises the complement of a nucleic acid molecule that encodes a TRAIL gene product. In a preferred embodiment, nucleic acid molecules that hybridize under conditions (a) and (b), above, encode gene products, e.g., gene products functionally equivalent to a TRAIL gene product.

In another embodiment the present invention includes any nucleotide sequence that hybridizes to a TRAIL nucleic acid of the invention under moderately stringent conditions, e.g. hybridization to filter-bound DNA in a buffer containing 6M urea at about 42° C. followed by one or more washes in 0.1×SSC/0.1% SDS at about 55° C.

Functionally equivalent TRAIL gene products include naturally occurring TRAIL gene products present in the same or different species. Functionally equivalent TRAIL gene products also include gene products that retain at least one of the biological activities of a TRAIL gene product, and/or which are recognized by and bind to antibodies (polyclonal or monoclonal) directed against such gene product.

Among the nucleic acid molecules of the invention are deoxyoligonucleotides (“oligos”) which hybridize under highly stringent or stringent conditions to the TRAIL nucleic acid molecules described above. In general, for probes between 14 and 70 nucleotides in length the melting temperature (Tm) is calculated using the formula: Tm(° C.)=81.5+16.6 (log [monovalent cations (molar)])+0.41 (% G+C) (500/N) where N is the length of the probe. If the hybridization is carried out in a solution containing formamide, the melting temperature is calculated using the equation Tm(° C.)=81.5+16.6 (log[monovalent cations (molar)])+0.41(%(G+C))−0.61(% formamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions may refer, e.g., to washing in 6×SSC/0.05% sodium pyrophosphate at 37° C. (for about 14-base oligos), 48° C. (for about 17-base oligos), 55° C. (for about 20-base oligos), and 60° C. (for about 23-base oligos).

The nucleic acid molecules of the invention further comprise the complements of the nucleic acids described above. Such molecules can, for example, act as antisense molecules, useful, for example, in TRAIL gene regulation, and/or as antisense primers in amplification reactions of TRAIL gene nucleic acid sequences.

The nucleic acid sequences of the invention may be used as part of ribozyme and/or triple helix sequences, also useful for TRAIL gene regulation. Still further, such molecules may be used as components of diagnostic methods whereby, for example, the presence of a particular TRAIL allele involving in an apoptosis-related disorder, e.g., cancer, may be detected, or whereby the methods involve mapping the human chromosomal region spanned by the alleles.

Fragments of the TRAIL nucleic acid molecules refer to TRAIL nucleic acid sequences that can be at least 10, 12, 15, 20, 30, 40, 50, 60, 70, 80, 90, 100, 200, 300, 400, 500, 600, 700, 800, 900, 1000, 1250, 1500, 1750, 2000, 2250, 2500, 2750, 3000, 3250, 3500, 3750, 4000, 4250, 4500, 4750, 5000, or more contiguous nucleotides in length. Alternatively, the fragments can comprise sequences that encode at least 10, 20, 30, 40, 50, 100, 150, 200, 250, 300, 350, 400, 450 or more contiguous amino acid residues of the TRAIL gene products. In one embodiment, the TRAIL nucleic acid molecules encode a gene product exhibiting at least one biological activity of a corresponding TRAIL gene product, e.g., a TRAIL gene product. Fragments of the TRAIL nucleic acid molecules can also refer to TRAIL exons or introns, and, further, to portions of TRAIL coding regions that encode domains of TRAIL gene products.

With respect to identification and isolation of TRAIL nucleotide sequences, such sequences can be readily obtained, for example, by utilizing standard sequencing and bacterial artificial chromosome (BAC) technologies.

As will be appreciated by those skilled in the art, DNA sequence polymorphisms of an TRAIL gene will exist within a population of individual organisms (e.g., within a human or canine population). Such polymorphisms may exist, for example, among individual organisms within a population due to natural allelic variation. Such polymorphisms include ones that lead to changes in an amino acid sequence. As used herein, the phrase “allelic variant” refers to a nucleotide sequence which occurs at a given locus or to a gene product encoded by that nucleotide sequence. Such natural allelic variations can result in 1-5%, 5-20%, or 20-50% variance in the nucleotide sequence of a given gene. An allele is one of a group of genes which occur alternatively at a given genetic locus. Alternative alleles can be identified by sequencing the gene of interest in a number of different individual organisms. This can be readily carried out by using hybridization probes to identify the same genetic locus in a variety of individual organisms. As used herein, the terms “gene” and “recombinant gene” refer to nucleic acid molecules comprising an open reading frame encoding a polypeptide of the invention. The terms “gene” and “recombinant gene” can further include nucleic acid molecules comprising upstream and/or exon/intron sequences and structures, i.e. secondary structures, such as stem-loop structures.

With respect to the cloning of additional allelic variants of the human TRAIL gene and homologs and orthologs from other species (e.g., guinea pig, cow, mouse, canine, feline), the isolated TRAIL gene sequences disclosed herein may be labeled and used to screen a cDNA library constructed from mRNA obtained from appropriate cells or tissues (e.g., spleen) derived from the organism (e.g., guinea pig, cow, mouse, canine, feline) of interest. The hybridization conditions used should generally be of a lower stringency when the cDNA library is derived from an organism different from the type of organism from which the labeled sequence was derived, and can routinely be determined based on, e.g., relative relatedness of the target and reference organisms.

Alternatively, the labeled fragment may be used to screen a genomic library derived from the organism of interest, again, using appropriately stringent conditions. Appropriate stringency conditions are well known to those of skill in the art as discussed above, and will vary predictably depending on the specific organisms from which the library and the labeled sequences are derived. For guidance regarding such conditions see, for example, Sambrook, et al., 1989, Molecular Cloning, A Laboratory Manual, Second Edition, Cold Spring Harbor Laboratory Press, N.Y.; and Ausubel, et al., 1989-1999, Current Protocols in Molecular Biology, Green Publishing Associates and Wiley Interscience, N.Y., both of which are incorporated herein by reference in their entirety.

Further, a TRAIL gene allelic variant may be isolated from, for example, human or canine nucleic acid, by performing PCR using two degenerate oligonucleotide primer pools designed on the basis of amino acid sequences within the TRAIL gene products disclosed herein. The template for the reaction may be cDNA obtained by reverse transcription of mRNA prepared from, for example, human or non-human cell lines or tissue known or suspected to express a wild type or mutant TRAIL gene allele (such as, for example, spleen cells).

The PCR product may be subcloned and sequenced to ensure that the amplified sequences represent the sequences of a TRAIL gene nucleic acid sequence. The PCR fragment may then be used to isolate a full length cDNA clone by a variety of methods. For example, the amplified fragment may be labeled and used to screen a bacteriophage cDNA library. Alternatively, the labeled fragment may be used to isolate genomic clones via the screening of a genomic library.

PCR technology also may be utilized to isolate full length cDNA sequences, as well as cDNA sequences corresponding to alternatively spliced mRNA species. For example, RNA may be isolated, following standard procedures, from an appropriate cellular or tissue source (i.e., one known, or suspected, to express the TRAIL gene, such as, spleen tissue samples obtained through biopsy or post-mortem). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNase H, and second strand synthesis may then be primed with a poly-C primer. Thus, cDNA sequences upstream of the amplified fragment may easily be isolated. For a review of cloning strategies that may be used, see e.g., Sambrook et al., 1989, supra, or Ausubel et al., supra.

A cDNA of an allelic, e.g., mutant, variant of the TRAIL gene may be isolated, for example, by using PCR, a technique that is well known to those of skill in the art. In this case, the first cDNA strand may be synthesized by hybridizing an oligo-dT oligonucleotide to mRNA isolated from tissue known or suspected to express a mutant TRAIL allele in an individual organism, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant TRAIL allele to that of the normal TRAIL allele, the mutation(s) responsible for the loss or alteration of function of the mutant TRAIL gene product can be ascertained.

Alternatively, a genomic library can be constructed using DNA obtained from an individual organism suspected of or known to carry a mutant TRAIL allele, or a cDNA library can be constructed using RNA from a tissue known, or suspected, to express a mutant TRAIL allele. An unimpaired TRAIL gene, or any suitable fragment thereof, may then be labeled and used as a probe to identify the corresponding mutant TRAIL allele in such libraries. Clones containing the mutant TRAIL gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant TRAIL allele in an individual organism suspected of or known to carry such a mutant allele. In this manner, gene products made by the putatively mutant tissue may be expressed and screened using standard antibody screening techniques in conjunction with antibodies raised against the normal TRAIL gene product, as described, below. (For screening techniques, see, for example, Harlow and Lane, eds., 1988, “Antibodies: A Laboratory Manual”, Cold Spring Harbor Laboratory Press, Cold Spring Harbor.)

In cases where a TRAIL mutation results in an expressed gene product with altered function (e.g., as a result of a missense or a frameshift mutation), a polyclonal set of anti-TRAIL gene product antibodies are likely to cross-react with the mutant TRAIL gene product. Library clones detected via their reaction with such labeled antibodies can be purified and subjected to sequence analysis according to methods well known to those of skill in the art.

TRAIL mutations or polymorphisms can further be detected using PCR amplification techniques. Primers can routinely be designed to amplify overlapping regions of the whole TRAIL sequence including the promoter regulating region. In one embodiment, primers are designed to cover the exon-intron boundaries such that, coding regions can be scanned for mutations.

The invention also includes nucleic acid molecules, preferably DNA molecules, that are the complements of the nucleotide sequences of the preceding paragraphs.

In certain embodiments, the nucleic acid molecules of the invention are present as part of nucleic acid molecules comprising nucleic acid sequences that contain or encode heterologous (e.g., vector, expression vector, or fusion protein) sequences.

TRAIL gene products include those gene products encoded by nucleic acid molecules comprising the TRAIL gene sequences described above. In addition, TRAIL gene products may include proteins that represent functionally equivalent gene products. Such an equivalent TRAIL gene product may contain deletions, including internal deletions, additions, including additions yielding fusion proteins, or substitutions of amino acid residues within and/or adjacent to the amino acid sequence encoded by the TRAIL gene sequences described above, but that result in a “silent” change, in that the change produces a functionally equivalent TRAIL gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Alternatively, where alteration of function is desired, deletion or non-conservative alterations can be engineered to produce altered TRAIL gene products. Such alterations can, for example, alter one or more of the biological functions of the TRAIL gene product. Further, such alterations can be selected so as to generate TRAIL gene products that are better suited for expression, scale up, etc. in the host cells chosen. For example, cysteine residues can be deleted or substituted with another amino acid residue in order to eliminate disulfide bridges.

Peptides and/or proteins corresponding to one or more domains of a TRAIL protein, as well as fusion proteins, in which a TRAIL protein or a portion of a TRAIL protein, such as a truncated TRAIL protein or peptide, or a TRAIL protein domain, is fused to an unrelated protein are also within the scope of this invention. Such proteins and peptides can be designed on the basis of the TRAIL nucleotide sequence disclosed, above, and/or on the basis of the TRAIL amino acid sequence disclosed herein. Fusion proteins include, but are not limited to, IgFc fusions which stabilize the TRAIL protein or peptide and prolong half life in vivo; or fusions to any amino acid sequence that allows the fusion proteins to be anchored to the cell membrane; or fusions of TRAIL protein domains to a marker which include, but are not limited to, an enzyme, a fluorescent protein, a luminescent protein, a flag or V5, a His epitope protein or peptide.

TRAIL proteins of the invention also include TRAIL protein sequences wherein domains encoded by at least one exon of the cDNA sequence, or fragments thereof, have been deleted.

The TRAIL protein sequences described above can include a domain which comprises a signal sequence that targets the TRAIL gene product for secretion. As used herein, a signal sequence includes a peptide of at least about 15 or 20 amino acid residues in length which occurs at the N-terminus of secretory and membrane-bound proteins and which contains at least about 70% hydrophobic amino acid residues such as alanine, leucine, isoleucine, phenylalanine, proline, tyrosine, tryptophan, or valine. In a preferred embodiment, a signal sequence contains at least about 10 to 40 amino acid residues, preferably about 19-34 amino acid residues, and has at least about 60-80%, more preferably 65-75%, and more preferably at least about 70% hydrophobic residues. A signal sequence serves to direct a protein containing such a sequence to a lipid bilayer.

A signal sequence of a polypeptide of the invention can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described TRAIL polypeptides having a signal sequence (that is, “immature” polypeptides), as well as to the TRAIL signal sequences themselves and to the TRAIL polypeptides in the absence of a signal sequence (i.e., the “mature” TRAIL cleavage products). It is to be understood that TRAIL polypeptides of the invention can further comprise polypeptides comprising any signal sequence having characteristics as described above and a mature TRAIL polypeptide sequence.

The TRAIL polypeptides of the invention can further comprise posttranslational modifications, including, but not limited to, glycosylations, acetylations, myristylations, and phosphorylations. If the native TRAIL protein does not have recognition motifs that allow such modifications, it would be routine for one skilled in the art to introduce into a TRAIL gene nucleotide sequences that encode motifs such as enzyme recognition signals so as to produce a modified TRAIL gene product.

The TRAIL gene products, peptide fragments thereof and fusion proteins thereof, may be produced by recombinant DNA technology using techniques well known in the art. Thus, methods for preparing the TRAIL gene polypeptides, peptides, fusion peptides and fusion polypeptides of the invention by expressing nucleic acid containing TRAIL gene sequences are described herein. Methods that are well known to those skilled in the art can be used to construct expression vectors containing TRAIL gene product coding sequences and appropriate transcriptional and translational control signals. These methods include, for example, in vitro recombinant DNA techniques, synthetic techniques, and in vivo genetic recombination. See, e.g., the techniques described in Sambrook, et al., 1989, supra, and Ausubel, et al., 1989, supra. Alternatively, RNA capable of encoding TRAIL gene product sequences may be chemically synthesized using, for example, synthesizers. See, e.g., the techniques described in “Oligonucleotide Synthesis”, 1984, Gait, ed., IRL Press, Oxford.

A variety of host-expression vector systems may be utilized to express the TRAIL gene coding sequences of the invention. Such host-expression systems represent vehicles by which the coding sequences of interest may be produced and subsequently purified, but also represent cells that may, when transformed or transfected with the appropriate nucleotide coding sequences, exhibit the TRAIL gene product of the invention in situ. These include, but are not limited to, microorganisms such as bacteria (e.g., E. coli, B. subtilis) transformed with recombinant bacteriophage DNA, plasmid DNA or cosmid DNA expression vectors containing TRAIL gene product coding sequences; yeast (e.g., Saccharomyces, Pichia) transformed with recombinant yeast expression vectors containing the TRAIL gene product coding sequences; insect cell systems infected with recombinant virus expression vectors (e.g., baculovirus) containing the TRAIL gene product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing TRAIL gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors can be advantageously selected depending upon the use intended for the TRAIL gene product to be expressed. For example, when a large quantity of such a protein is to be produced, for the generation of pharmaceutical compositions of TRAIL protein or for raising antibodies to TRAIL protein, for example, vectors that direct the expression of high levels of fusion protein products that are readily purified are desirable. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica, nuclear polyhedrosis virus (AcNPV) is used as a vector to express foreign genes. The virus grows in Spodoptera frugiperda cells. TRAIL gene coding sequences can be cloned individually into non-essential regions (for example the polyhedrin gene) of the virus and placed under control of an AcNPV promoter (for example the polyhedrin promoter). Successful insertion of TRAIL gene coding sequences will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith, et al., 1983, J. Virol. 46, 584; Smith, U.S. Pat. No. 4,215,051).

In mammalian host cells, a number of viral-based expression systems can be utilized. In cases where an adenovirus is used as an expression vector, a TRAIL gene coding sequence of interest may be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing TRAIL gene product in infected hosts. (e.g., See Logan and Shenk, 1984, Proc. Natl. Acad. Sci. USA 81, 3655-3659). Specific initiation signals may also be required for efficient translation of inserted TRAIL gene product coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner, et al., 1987, Methods in Enzymol. 153, 516-544).

In addition, a host cell strain can be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include but are not limited to CHO, VERO, BHK, HeLa, COS, MDCK, 293, 3T3, and WI38.

For long-term, high-yield production of recombinant proteins, stable expression is preferred. For example, cell lines that stably express the TRAIL gene product can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells are allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method can advantageously be used to engineer cell lines that express the TRAIL gene product. Such engineered cell lines may be particularly useful in screening and evaluation of compounds that affect the endogenous activity of the TRAIL gene product.

A number of selection systems can be used, including, but not limited to, the herpes simplex virus thymidine kinase (Wigler, et al., 1977, Cell 11, 223), hypoxanthine-guanine phosphoribosyltransferase (Szybalska and Szybalski, 1962, Proc. Natl. Acad. Sci. USA 48, 2026), and adenine phosphoribosyltransferase (Lowy, et al., 1980, Cell 22, 817) genes can be employed in tk-, hgprt- or aprt-cells, respectively. Also, antimetabolite resistance can be used as the basis of selection for the following genes: dhfr, which confers resistance to methotrexate (Wigler, et al., 1980, Natl. Acad. Sci. USA 77, 3567; O'Hare, et al., 1981, Proc. Natl. Acad. Sci. USA 78, 1527); gpt, which confers resistance to mycophenolic acid (Mulligan and Berg, 1981, Proc. Natl. Acad. Sci. USA 78, 2072); neo, which confers resistance to the aminoglycoside G-418 (Colberre-Garapin, et al., 1981, J. Mol. Biol. 150, 1); and hygro, which confers resistance to hygromycin (Santerre, et al., 1984, Gene 30, 147).

Alternatively, any fusion protein can be readily purified by utilizing an antibody specific for the fusion protein being expressed. For example, a system described by Janknecht, et al. allows for the ready purification of non-denatured fusion proteins expressed in human cell lines (Janknecht, et al., 1991, Proc. Natl. Acad. Sci. USA 88, 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

Alternatively, the expression characteristics of an endogenous TRAIL gene within a cell, cell line, or microorganism can be modified by inserting a heterologous DNA regulatory element into the genome of a stable cell line or cloned microorganism such that the inserted regulatory element is operatively linked with the endogenous TRAIL gene. For example, an endogenous TRAIL gene which is normally “transcriptionally silent”, i.e., a TRAIL gene which is normally not expressed, or is expressed only at very low levels in a cell, cell line, or microorganism, may be activated by inserting a regulatory element which is capable of promoting the expression of a normally expressed gene product in that cell, cell line, or microorganism. Alternatively, a transcriptionally silent, endogenous TRAIL gene can be activated by insertion of a promiscuous regulatory element that works across cell types.

A heterologous regulatory element can be inserted into a stable cell line or cloned microorganism, such that it is operatively linked to an endogenous TRAIL gene, using techniques, such as targeted homologous recombination, which are well known to those of skill in the art, and described e.g., in Chappel, U.S. Pat. No. 5,272,071; PCT publication No. WO 91/06667, published May 16, 1991.

TRAIL gene products also can be expressed in transgenic animals. Animals of any species, including, but not limited to, mice, rats, rabbits, guinea pigs, pigs, micro-pigs, goats, sheep, and non-human primates, e.g., baboons, monkeys, and chimpanzees can be used to generate TRAIL transgenic animals. The term “transgenic,” as used herein, refers to animals expressing TRAIL gene sequences from a different species (e.g., mice expressing human or canine TRAIL sequences), as well as animals that have been genetically engineered to overexpress endogenous (i.e., same species) TRAIL sequences, or animals that have been genetically engineered to no longer express endogenous TRAIL gene sequences (i.e., “knock-out” animals), and their progeny.

Any technique known in the art can be used to introduce a TRAIL gene transgene into animals to produce the founder lines of transgenic animals. Such techniques include, but are not limited to, pronuclear microinjection (Hoppe and Wagner, 1989, U.S. Pat. No. 4,873,191); retrovirus mediated gene transfer into germ lines (van der Putten, et al., 1985, Proc. Natl. Acad. Sci., USA 82, 6148-6152); gene targeting in embryonic stem cells (Thompson, et al., 1989, Cell 56, 313-321); electroporation of embryos (Lo, 1983, Mol. Cell. Biol. 3, 1803-1814); and sperm-mediated gene transfer (Lavitrano et al., 1989, Cell 57, 717-723). (For a review of such techniques, see Gordon, 1989, Transgenic Animals, Intl. Rev. Cytol. 115, 171-229.)

Any technique known in the art can be used to produce transgenic animal clones containing a TRAIL transgene, for example, nuclear transfer into enucleated oocytes of nuclei from cultured embryonic, fetal or adult cells induced to quiescence (Campbell, et al., 1996, Nature 380, 64-66; Wilmut, et al., Nature 385, 810-813).

The present invention provides transgenic animals that carry a TRAIL transgene in all their cells, as well as animals that carry the transgene in some, but not all their cells, i.e., mosaic animals. The transgene can be integrated as a single transgene or in concatamers, e.g., head-to-head tandems or head-to-tail tandems. The transgene can also be selectively introduced into and activated in a particular cell type by following, for example, the teaching of Lasko et al. (Lasko, et al., 1992, Proc. Natl. Acad. Sci. USA 89, 6232-6236). The regulatory sequences required for such a cell-type specific activation will depend upon the particular cell type of interest, and will be apparent to those of skill in the art. When it is desired that the TRAIL gene transgene be integrated into the chromosomal site of the endogenous TRAIL gene, gene targeting is preferred. Briefly, when such a technique is to be utilized, vectors containing some nucleotide sequences homologous to the endogenous TRAIL gene are designed for the purpose of integrating, via homologous recombination with chromosomal sequences, into and disrupting the function of the nucleotide sequence of the endogenous TRAIL gene. The transgene can also be selectively introduced into a particular cell type, thus inactivating the endogenous TRAIL gene in only that cell type. The regulatory sequences required for such a cell-type specific inactivation will depend upon the particular cell type of interest, and will be apparent to those of skill in the art.

Once transgenic animals have been generated, the phenotypic expression of the recombinant TRAIL gene may be assayed utilizing standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques that include, but are not limited to, Northern blot analysis of tissue samples obtained from the animal, in situ hybridization analysis, and RT-PCR (reverse transcriptase PCR). Samples of TRAIL gene-expressing tissue, can also be evaluated immunocytochemically using antibodies specific for the TRAIL transgene product.

TRAIL gene products, or peptide fragments thereof, can be prepared for a variety of uses. For example, such gene products, or peptide fragments thereof, can be used for the generation of antibodies, in diagnostic assays, or for mapping and the identification of other cellular or extracellular gene products involved in the regulation of an angiogenesis-related disorder, such as cancer. Such TRAIL gene products include, but are not limited to, soluble derivatives such as peptides or polypeptides corresponding to one or more domains of the TRAIL gene product, particularly TRAIL gene products that are modified such that they are deleted for one or more hydrophobic domains. Alternatively, antibodies to the TRAIL protein or anti-idiotypic antibodies that mimic the TRAIL gene product (including Fab fragments), antagonists or agonists can be used to treat angiogenesis-related disorders, such as cancer. In yet another approach, nucleotide constructs encoding such TRAIL gene products can be used to genetically engineer host cells to express such TRAIL gene products in vivo; these genetically engineered cells can function as “bioreactors” in the body delivering a continuous supply of TRAIL gene product, TRAIL peptides or soluble TRAIL polypeptides.

The present invention provides methods for the production of antibodies capable of specifically recognizing one or more TRAIL gene product epitopes or epitopes of conserved variants or peptide fragments of the gene products.

Such antibodies can include, but are not limited to, polyclonal antibodies, monoclonal antibodies (mAbs), canine and caninized. Or chimeric antibodies, single chain antibodies, Fab fragments, F(ab′)2 fragments, fragments produced by a Fab expression library, anti-idiotypic (anti-Id) antibodies, and epitope-binding fragments of any of the above. Such antibodies may be used, for example, in the detection of a TRAIL gene product in a biological sample and can, therefore, be utilized as part of a diagnostic or prognostic technique whereby subjects can be tested for abnormal levels of TRAIL gene products, and/or for the presence of abnormal forms of such gene products. Such antibodies can also be utilized in conjunction with, for example, compound screening schemes, as described below, for the evaluation of the effect of test compounds on TRAIL gene product levels and/or activity. Additionally, such antibodies can be used in conjunction with the gene therapy techniques described below, for example, to evaluate the normal and/or engineered TRAIL -expressing cells prior to their introduction into the subject.

Anti-TRAIL gene product antibodies can additionally be used as a method for the inhibition of abnormal TRAIL gene product activity. Thus, such antibodies can be utilized as part of treatment methods for an apoptosis-related disorder, e.g., cancer.

For the production of antibodies against a TRAIL gene product, various host animals can be immunized by injection with a TRAIL gene product, or a portion thereof. Such host animals can include, but are not limited to, rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a TRAIL gene product, or an antigenic functional derivative thereof. For the production of polyclonal antibodies, host animals such as those described above, can be immunized by injection with a TRAIL gene product supplemented with adjuvants as also described above.

Monoclonal antibodies, which are homogeneous populations of antibodies to a particular antigen, can be obtained by any technique that provides for the production of antibody molecules by continuous cell lines in culture. These include, but are not limited to, the hybridoma technique of Kohler and Milstein, (1975, Nature 256, 495-497; and U.S. Pat. No. 4,376,110), the human B-cell hybridoma technique (Kosbor et al., 1983, Immunology Today 4, 72; Cole et al., 1983, Proc. Natl. Acad. Sci. USA 80, 2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

Additionally, recombinant antibodies, such as chimeric and humanized monoclonal antibodies, comprising both canine and non-canine portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816397, which are incorporated herein by reference in their entirety.) Caninized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and caninized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Fremont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

Alternatively, techniques described for the production of single chain antibodies (U.S. Pat. No. 4,946,778; Bird, 1988, Science 242, 423-426; Huston, et al., 1988, Proc. Natl. Acad. Sci. USA 85, 5879-5883; and Ward, et al., 1989, Nature 334, 544-546) can be adapted to produce single chain antibodies against TRAIL gene products. Single chain antibodies are formed by linking the heavy and light chain fragments of the Fv region via an amino acid bridge, resulting in a single chain polypeptide.

Antibody fragments that recognize specific epitopes can be generated by known techniques. For example, such fragments include, but are not limited to, the F(ab′)2 fragments, which can be produced by pepsin digestion of the antibody molecule and the Fab fragments, which can be generated by reducing the disulfide bridges of the F(ab′)2 fragments. Alternatively, Fab expression libraries may be constructed (Huse, et al., 1989, Science, 246, 1275-1281) to allow rapid and easy identification of monoclonal Fab fragments with the desired specificity.

Described herein are various applications of TRAIL gene sequences, TRAIL gene products, including peptide fragments and fusion proteins thereof, and of antibodies directed against TRAIL gene products and peptide fragments thereof. Such applications include, for example, prognostic and diagnostic evaluation of an angiogenesis-related disorder, e.g., cancer, and the identification of subjects with a predisposition to such disorders, as described herein. Additionally, such applications include methods for the identification of compounds that modulate the expression of a TRAIL gene and/or the synthesis or activity of a TRAIL gene product, as described below, and for the treatment of an apoptosis-related disorder, e.g. cancer, as described, below.

In addition, TRAIL gene sequences and gene products, including peptide fragments and fusion proteins thereof, and antibodies directed against TRAIL gene products and peptide fragments thereof, have applications for purposes independent of the role TRAIL may have in apoptosis-related disorders and processes. For example, TRAIL gene products, including peptide fragments, as well as TRAIL -specific antibodies, can be used for construction of fusion proteins to facilitate recovery, detection, or localization of another protein of interest. In addition, TRAIL genes and gene products can be used for genetic mapping, i.e., refining the genetic map. For example, antibodies specific to TRAIL can be used as probes to detect expression of human TRAIL in somatic cell hybrids containing human chromosomes, or portions of human chromosomes. Such TRAIL-specific antibodies can be used to identify cells that contain the TRAIL chromosomal region. This method can be used, for example, to localize a gene or trait of interest to this region of the chromosome.

TRAIL gene sequences and gene products can be used for mapping and refining a chromosomal map. The TRAIL sequence can be used to develop new genetic markers to further refine chromosomal intervals that are associated with various apoptosis-related disorders, including, but not limited to, cancer. As will be apparent to the skilled artisan, nucleic acid sequences within a genetic interval associated with a disease can be scanned for new markers, such as microsatellites. Microsatellites, also known as simple-sequence repeats (SSRs), are hypervariable tandem-sequence repeats consisting of di-, tri-, or tetranucleotide repeats of 1-5 nucleotides. Such microsatellites make excellent genetic markers for linkage studies since they are distributed ubiquitously throughout the human genome, are highly variable in repeat length, and tend to be highly polymorphic. Relatively common microsatellites (e.g., (CA)n dinucleotide repeats) occur approximately every 300-500 kb. In addition to microsatellite repeats, the region can be scanned for other types of polymorphic sites useful for fine mapping, such as minisatellites (9-64 nucleotide repeats), restriction fragment length polymorphisms (RFLPs), and single nucleotide polymorphisms, which occur much less frequently. Once a polymorphic site is identified in a new sequence, PCR primers that flank the polymorphic site can be synthesized and used to amplify the microsatellite or other polymorphic site. The length of the repeat can then determined by resolving the PCR product on a polyacrylamide sequencing gel. Genomic DNA from human populations can then be analyzed for the simple-sequence length polymorphisms (SSLPs) to determine the frequency and variability of the repeat. Once a high quality SSLP is found, linkage analysis can be performed on an affected population to determine whether an angiogenesis-related disorder, such as cancer, is linked to the new marker. Other techniques, such as Southern blot hybridization and ligase-chain reaction (LCR), can be used in addition to, or in conjunction with, PCR-based methods to analyze polymorphisms in genomic populations (see, Current Protocols in Human Genetics, Dracopoli et al. (eds.) John Wiley & Sons, 1998).

In another embodiment, a TRAIL gene, protein or a fragment or domain thereof, can be used for construction of fusion proteins. Finally, TRAIL nucleic acids and gene products have generic uses, such as supplemental sources of nucleic acids, proteins and amino acids for food additives or cosmetic products.

Portions or fragments of the cDNA sequences identified herein (and the corresponding complete gene sequences) can be used in numerous ways as polynucleotide reagents. For example, these sequences can be used to: (i) screen for TRAIL gene-specific mutations or polymorphisms, (ii) map their respective genes on a chromosome and, thus, locate gene regions associated with genetic disease; (iii) identify an individual organism from a minute biological sample (tissue typing); and (iv) aid in forensic identification of a biological sample. These applications are described in the subsections below.

A variety of methods can be employed to screen for the presence of TRAIL gene-specific mutations or polymorphisms (including polymorphisms flanking a TRAIL gene, e.g., ones that cosegregate with a particular TRAIL allele) and to detect and/or assay levels of TRAIL nucleic acid sequences.

Mutations or polymorphisms within or flanking the TRAIL gene can be detected by utilizing a number of techniques. Nucleic acid from any nucleated cell, or any cell that expresses the TRAIL gene of interest, can be used as the starting point for such assay techniques, and may be isolated according to standard nucleic acid preparation procedures that are well known to those of skill in the art.

TRAIL nucleic acid sequences can be used in hybridization or amplification assays of biological samples to detect abnormalities involving TRAIL gene structure, including point mutations, insertions, deletions, inversions, translocations and chromosomal rearrangements. Such assays can include, but are not limited to, Southern analyses, single-stranded conformational polymorphism analyses (SSCP), and PCR analyses.

Diagnostic methods for the detection of TRAIL gene-specific mutations or polymorphisms can involve, for example, contacting and incubating nucleic acids obtained from a sample, e.g., derived from a patient sample or other appropriate cellular source with one or more labeled nucleic acid reagents including recombinant DNA molecules, cloned genes or degenerate variants thereof, such as described, above, under conditions favorable for the specific annealing of these reagents to their complementary sequences within or flanking the TRAIL gene. The diagnostic methods of the present invention further encompass contacting and incubating nucleic acids for the detection of single nucleotide mutations or polymorphisms of the TRAIL gene. Preferably, these nucleic acid reagent sequences within the TRAIL gene, or chromosome nucleotide sequences flanking the TRAIL gene are 15 to 30 nucleotides in length.

After incubation, all non-annealed nucleic acids are removed from the nucleic acid: TRAIL molecule hybrid. The presence of nucleic acids that have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents of the type described above, are easily removed. Detection of the remaining, annealed, labeled TRAIL nucleic acid reagents is accomplished using standard techniques well-known to those in the art. The TRAIL gene sequences to which the nucleic acid reagents have annealed can be compared to the annealing pattern expected from a normal TRAIL gene sequence in order to determine whether a TRAIL gene mutation is present.

In a preferred embodiment, TRAIL mutations or polymorphisms can be detected by using a microassay TRAIL nucleic acid sequences immobilized to a substrate or “gene chip” (see, e.g. Cronin et al., 1996, Human Mutation 7:244-255).

Alternative diagnostic methods for the detection of TRAIL gene-specific nucleic acid molecules (or TRAIL flanking sequences), in patient samples or other appropriate cell sources, can involve their amplification, e.g., by PCR (the experimental embodiment set forth in Mullis, 1987, U.S. Pat. No. 4,683,202), followed by the analysis of the amplified molecules using techniques well known to those of skill in the art, such as, those listed above. The resulting amplified sequences can be compared to those that would be expected if the nucleic acid being amplified contained only normal copies of the TRAIL gene in order to determine whether a TRAIL gene mutation or polymorphism in linkage disequilibrium with a disease-causing TRAIL allele exists.

Additionally, well-known genotyping techniques can be performed to identify individual organisms carrying TRAIL gene mutations. Such techniques include, for example, the use of restriction fragment length polymorphisms (RFLPs), which involve sequence variations in one of the recognition sites for the specific restriction enzyme used.

Further, improved methods for analyzing DNA polymorphisms, which can be utilized for the identification of TRAIL gene-specific mutations, have been described that capitalize on the presence of variable numbers of short, tandemly repeated DNA sequences between the restriction enzyme sites. For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers that are so closely spaced exhibit a high frequency co-inheritance, and are extremely useful in the identification of genetic mutations, such as, for example, mutations within the TRAIL gene, and the diagnosis of diseases and disorders related to TRAIL mutations.

Also, Caskey et al. (U.S. Pat. No. 5,364,759) describe a DNA profiling assay for detecting short tri and tetra nucleotide repeat sequences. The process includes extracting the DNA of interest, such as the TRAIL gene, amplifying the extracted DNA, and labeling the repeat sequences to form a genotypic map of the individual organism's DNA.

Other methods well known in the art can be used to identify single nucleotide polymorphisms (SNPs), including biallelic SNPs or biallelic markers which have two alleles, both of which are present at a fairly high frequency in a population. Conventional techniques for detecting SNPs include, e.g., conventional dot blot analysis, SSCP analysis (see, e.g., Orita et al., 1989, Proc. Natl. Acad. Sci. USA 86:2766-2770), denaturing gradient gel electrophoresis (DGGE), heteroduplex analysis, mismatch cleavage detection, and other routine techniques well known in the art (see, e.g., Sheffield et al., 1989, Proc. Natl. Acad. Sci. 86:5855-5892; Grompe, 1993, Nature Genetics 5:111-117). Alternative, preferred methods of detecting and mapping SNPs involve microsequencing techniques wherein an SNP site in a target DNA is detecting by a single nucleotide primer extension reaction (see, e.g., Goelet et al., PCT Publication No. WO92/15712; Mundy, U.S. Pat. No. 4,656,127; Vary and Diamond, U.S. Pat. No. 4,851,331; Cohen et al., PCT Publication No. WO91/02087; Chee et al., PCT Publication No. WO95/11995; Landegren et al., 1988, Science 241:1077-1080; Nicerson et al., 1990, Proc. Natl. Acad. Sci. U.S.A. 87:8923-8927; Pastinen et al.,1997, Genome Res. 7:606-614; Pastinen et al., 1996, Clin. Chem. 42:1391-1397; Jalanko et al., 1992, Clin. Chem. 38:39-43; Shumaker et al., 1996, Hum. Mutation 7:346-354; Caskey et al., PCT Publication No. WO 95/00669).

The level of TRAIL gene expression also can be assayed. For example, RNA from a cell type or tissue, such as spleen, known, or suspected, to express the TRAIL gene, can be isolated and tested utilizing hybridization or PCR techniques such as are described, above. The isolated cells can be derived from cell culture or from a subject. The analysis of cells taken from culture may be a necessary step in the assessment of cells to be used as part of a cell-based gene therapy technique or, alternatively, to test the effect of compounds on the expression of the TRAIL gene. Such analyses may reveal both quantitative and qualitative aspects of the expression pattern of the TRAIL gene, including activation or inactivation of TRAIL gene expression.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the TRAIL gene nucleic acid reagents described above. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such TRAIL gene expression assays in situ, i.e., directly upon tissue sections (fixed and/or frozen) of subject tissue obtained from biopsies or resections, such that no nucleic acid purification is necessary. Nucleic acid reagents such as those described above, can be used as probes and/or primers for such in situ procedures (see, for example, Nuovo, G. J., 1992, “PCR In Situ Hybridization: Protocols And Applications”, Raven Press, N.Y.).

Alternatively, if a sufficient quantity of the appropriate cells can be obtained, standard Northern analysis can be performed to determine the level of mRNA expression of the TRAIL gene.

Once the sequence (or a portion of the sequence) of a gene has been isolated, this sequence can be used to map the location of the gene on a chromosome. Accordingly, nucleic acid molecules described herein, or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus simplifying the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al., 1983, Science, 220:919-924.

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sublocalization can be achieved with panels of fragments from specific chromosomes. Other mapping strategies which can similarly be used to map a gene to its chromosome include in situ hybridization (described in Fan et al., 1990, Proc. Natl. Acad. Sci. USA 87:6223-27), pre-screening with labeled flow-sorted chromosomes (Popp S, et al., 1993, Hum Genet., 92(6):527-32) and pre-selection by hybridization to chromosome specific cDNA libraries. Fluorescence in situ hybridization (FISH) of a DNA sequence to a metaphase chromosomal spread can further be used to provide a precise chromosomal location in one step. (For a review of this technique, see Verma et al., Human Chromosomes: A Manual of Basic Techniques, Pergamon Press, New York, 1988.)

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al.,1987, Nature 325:783-787.

Moreover, differences in the DNA sequences between individual organisms affected and unaffected with a disease associated with a gene of the invention can be determined. If a mutation is observed in some or all of the affected individual organisms but not in any unaffected individual organisms, then the mutation is likely to be the causative agent of the particular disease. Comparison of affected and unaffected individual organisms generally involves first looking for structural alterations in the chromosomes such as deletions or translocations that are visible from chromosome spreads or detectable using PCR based on that DNA sequence. Ultimately, complete sequencing of genes from several individual organisms can be performed to confirm the presence of a mutation and to distinguish mutations from polymorphisms.

The nucleic acid sequences of the present invention also can be used to identify individual organisms from minute biological samples. The United States military, for example, is considering the use of restriction fragment length polymorphism (RFLP) for identification of its personnel. In this technique, an individual organism's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual organism's genome. Thus, the nucleic acid sequences described herein can be used to prepare two PCR primers from the 5′ and 3′ ends of the sequences. These primers can then be used to amplify an individual organism's DNA and subsequently sequence it.

Panels of corresponding DNA sequences from individual organisms, prepared in this manner, can provide unique individual identifications, as each individual organism will have a unique set of such DNA sequences due to allelic differences. The sequences of the present invention can be used to obtain such identification sequences from individual organisms and from tissues. The nucleic acid sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual organism can be compared for identification purposes. Because greater numbers of polymorphisms occur in the noncoding regions, fewer sequences are necessary to differentiate individuals.

If a panel of reagents from the nucleic acid sequences described herein is used to generate a unique identification database for an individual, those same reagents can later be used to identify tissue from that individual. Using the unique identification database, positive identification of the individual organism, living or dead, can be made from extremely small tissue samples.

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual organism). As mentioned above, actual base sequence information can be used for identification as an accurate alternative to patterns formed by restriction enzyme generated fragments. Sequences targeted to noncoding regions are particularly appropriate for this use as greater numbers of polymorphisms occur in the noncoding regions, making it easier to differentiate individual organisms using this technique. Examples of polynucleotide reagents include the nucleic acid sequences of the invention or portions thereof, e.g., fragments derived from noncoding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., liver tissue. This can be very useful in cases where a forensic pathologist is presented with a tissue of unknown origin. Panels of such probes can be used to identify tissue by species and/or by organ type.

Antibodies directed against unimpaired or mutant TRAIL gene products or conserved variants or peptide fragments thereof, which are discussed, above, can also be used as diagnostics and prognostics for an angiogenesis-related disorder, e.g., cancer, as described herein. Such methods can be used to detect abnormalities in the level of TRAIL gene product synthesis or expression, or abnormalities in the structure, temporal expression, and/or physical location of TRAIL gene product. The antibodies and immunoassay methods described below have, for example, important in vitro applications in purifying TRAIL gene products and in assessing the efficacy of treatments for angiogenesis-related disorders, e.g., cancer. Antibodies, or fragments of antibodies, such as those described below, can be used to screen potentially therapeutic compounds in vitro to determine their effects on TRAIL gene expression and TRAIL peptide production. The compounds that have beneficial effects on an angiogenesis-related disorder, e.g., cancer, can be identified, and a therapeutically effective dose determined.

In vitro immunoassays can also be used, for example, to assess the efficacy of cell-based gene therapy for an angiogenesis-related disorder, e.g., cancer. Antibodies directed against TRAIL peptides can be used in vitro to determine, for example, the level of TRAIL gene expression achieved in cells genetically engineered to produce TRAIL peptides. In the case of intracellular TRAIL gene products, such an assessment is done, preferably, using cell lysates or extracts. Such analysis allows for a determination of the number of transformed cells necessary to achieve therapeutic efficacy in vivo, as well as optimization of the gene replacement protocol.

The tissue or cell type to be analyzed will generally include those that are known, or suspected, to express the TRAIL gene. The protein isolation methods employed herein can, for example, be such as those described in Harlow and Lane (1988, “Antibodies: A Laboratory Manual”, Cold Spring Harbor Laboratory Press, Cold Spring Harbor, N.Y.). The isolated cells can be derived from cell culture or from a subject. The analysis of cells taken from culture may be a necessary step in the assessment of cells to be used as part of a cell-based gene therapy technique or, alternatively, to test the effect of compounds on the expression of the TRAIL gene.

Preferred diagnostic methods for the detection of TRAIL gene products or conserved variants or peptide fragments thereof, can involve, for example, immunoassays wherein the TRAIL gene products or conserved variants or peptide fragments are detected by their interaction with an anti-TRAIL gene product-specific antibody.

For example, antibodies, or fragments of antibodies, such as those described, above, useful in the present invention can be used to quantitatively or qualitatively detect the presence of TRAIL gene products or conserved variants or peptide fragments thereof. This can be accomplished, for example, by immunofluorescence techniques employing a fluorescently labeled antibody (see below, this section) coupled with light microscopic, flow cytometric, or fluorimetric detection. Such techniques are especially preferred for TRAIL gene products that are expressed on the cell surface.

The antibodies (or fragments thereof) useful in the present invention can, additionally, be employed histologically, as in immunofluorescence or immunoelectron microscopy, for in situ detection of TRAIL gene products or conserved variants or peptide fragments thereof. In situ detection may be accomplished by removing a histological specimen from a subject, and applying thereto a labeled antibody of the present invention. The antibody (or fragment) is preferably applied by overlaying the labeled antibody (or fragment) onto a biological sample. Through the use of such a procedure, it is possible to determine not only the presence of the TRAIL gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for TRAIL gene products or conserved variants or peptide fragments thereof will typically comprise incubating a sample, such as a biological fluid, a tissue extract, freshly harvested cells, or lysates of cells, that have been incubated in cell culture, in the presence of a detectably labeled antibody capable of identifying TRAIL gene products or conserved variants or peptide fragments thereof, and detecting the bound antibody by any of a number of techniques well-known in the art.

The biological sample can be brought in contact with and immobilized onto a solid phase support or carrier such as nitrocellulose, or other solid support that is capable of immobilizing cells, cell particles or soluble proteins. The support can then be washed with suitable buffers followed by treatment with the detectably labeled TRAIL gene specific antibody. The solid phase support can then be washed with the buffer a second time to remove unbound antibody. The amount of bound label on solid support can then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-TRAIL gene product antibody can be determined according to well known methods. Those skilled in the art will be able to determine operative and optimal assay conditions for each determination by employing routine experimentation.

One of the ways in which the TRAIL gene peptide-specific antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, β-glycerophosphate, dehydrogenase, triose phosphate isomerase, horseradish peroxidase, alkaline phosphatase, asparaginase, glucose oxidase, .-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection also can be accomplished using any of a variety of other immunoassays. For example, by radioactively labeling the antibodies or antibody fragments, it is possible to detect TRAIL gene peptides through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The following assays are designed to identify compounds that bind to a TRAIL gene product, proteins, e.g., intracellular proteins or portions of proteins that interact with an TRAIL gene product, compounds that interfere with the interaction of an TRAIL gene product with intracellular proteins and compounds that modulate the activity of an TRAIL gene (i.e., modulate the level of TRAIL gene expression and/or modulate the level of TRAIL gene product activity). Assays can additionally be utilized that identify compounds that bind to TRAIL gene regulatory sequences (e.g., promoter sequences; see e.g., Platt, 1994, J. Biol. Chem. 269, 28558-28562), and that can modulate the level of TRAIL gene expression. Compounds can include, but are not limited to, small organic molecules, such as ones that are able to cross the blood-brain barrier, gain entry into an appropriate cell and affect expression of the TRAIL gene or some other gene involved in an TRAIL regulatory pathway, or intracellular proteins.

Methods for the identification of such intracellular proteins are described, below. Such intracellular proteins may be involved in the control and/or regulation of mood. Further, among these compounds are compounds that affect the level of TRAIL gene expression and/or TRAIL gene product activity and that can be used in the therapeutic treatment of TRAIL disorders, e.g., cancer, as described, below.

Compounds can include, but are not limited to, peptides such as, for example, soluble peptides, including, but not limited to, Ig-tailed fusion peptides, and members of random peptide libraries; (see, e.g., Lam, et al., 1991, Nature 354, 82-84; Houghten, et al., 1991, Nature 354, 84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72, 767-778), antibodies (including, but not limited to, polyclonal, monoclonal, human, humanized, anti-idiotypic, chimeric or single chain antibodies, and FAb, F(ab′)2 and FAb expression library fragments, and epitope-binding fragments thereof), and small organic or inorganic molecules.

Such compounds can also comprise compounds, in particular drugs or members of classes or families of drugs, known to ameliorate or exacerbate the symptoms of an apoptosis-related disorder. Such compounds include, but are not limited to, angiogenesis inhibitors: metalloproteinase inhibitors, FGF and VEGF receptor inhibitors, COX-2 inhibitors, INF, IL-12, Taxol, vinblastine, thalidomide etc. Preferably such compounds are utilized in a manner (e.g., different dosage, mode of administration, and/or co-administration with one or more additional compounds) that differs from the manner in which such compounds have been administered previously.

Compounds identified via assays such as those described herein can be useful, for example, in elaborating the biological function of the TRAIL gene product, and for ameliorating apoptosis-related disorders, e.g., cancer. For example, compounds identified via such techniques can provide lead compounds to be tested for an ability to modulate a TRAIL-mediated process and/or to ameliorate symptoms of a apoptosis-related disorder. Assays for testing the effectiveness of compounds, identified by, for example, techniques such as those described above, are discussed, below.

In vitro systems can be designed to identify compounds that bind TRAIL gene products of the invention, such as, for example, TRAIL polypeptides. Compounds identified can be useful, for example, in modulating the activity of unimpaired and/or mutant TRAIL gene products, can be useful in elucidating the biological function of the TRAIL gene product, can be utilized in screens for identifying compounds that disrupt normal TRAIL gene product interactions, or can in themselves disrupt such interactions, and can provide lead compounds to be further tested for an ability to modulate a TRAIL-mediated process and/or to ameliorate symptoms of an apoptosis-related disorder.

The principle of the assays used to identify compounds that bind to TRAIL gene products involves preparing a reaction mixture of the TRAIL gene product and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring a TRAIL gene product or the test substance onto a solid phase and detecting TRAIL gene product/test compound complexes anchored on the solid phase at the end of the reaction. In one embodiment of such a method, the TRAIL gene product can be anchored onto a solid surface, and the test compound, which is not anchored, can be labeled, either directly or indirectly.

In practice, microtiter plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for TRAIL gene product or the test compound to anchor any complexes formed in solution, and a labeled antibody specific for the other component of the possible complex to detect anchored complexes.

Any method suitable for detecting protein-protein interactions can be employed for identifying TRAIL protein-protein interactions.

Among the traditional methods that can be employed are co-immunoprecipitation, cross-linking and co-purification through gradients or chromatographic columns. Utilizing procedures such as these allows for the identification of proteins, including intracellular proteins, that interact with TRAIL gene products. Once isolated, such a protein can be identified and can be used in conjunction with standard techniques, to identify proteins it interacts with. For example, at least a portion of the amino acid sequence of a protein that interacts with the TRAIL gene product can be ascertained using techniques well known to those of skill in the art, such as via the Edman degradation technique (see, e.g., Creighton, 1983, “Proteins: Structures and Molecular Principles,” W.H. Freeman & Co., N.Y., pp.34-49). The amino acid sequence obtained can be used as a guide for the generation of oligonucleotide mixtures that can be used to screen for gene sequences encoding such proteins. Screening made be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Additionally, methods can be employed that result in the simultaneous identification of genes that encode a protein which interacts with an TRAIL protein. These methods include, for example, probing expression libraries with labeled TRAIL protein, using TRAIL protein in a manner similar to the well known technique of antibody probing of lambda gt11 libraries.

One method that detects protein interactions in vivo, the two-hybrid system, is described in detail for illustration only and not by way of limitation. One version of this system has been described (Chien, et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to the TRAIL gene product and the other consists of the transcription activator protein's activation domain fused to an unknown protein that is encoded by a cDNA that has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology can be used to screen activation domain libraries for proteins that interact with the “bait” gene product. By way of example, and not by way of limitation, TRAIL gene products can be used as the bait gene product. Total genomic or cDNA sequences are fused to the DNA encoding an activation domain. This library and a plasmid encoding a hybrid of a bait TRAIL gene product fused to the DNA-binding domain are co-transformed into a yeast reporter strain, and the resulting transformants are screened for those that express the reporter gene. For example, and not by way of limitation, a bait TRAIL gene sequence, such as the open reading frame of the TRAIL gene, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with the bait TRAIL gene product can be made using methods routinely practiced in the art. According to the particular system described herein, for example, the cDNA fragments can be inserted into a vector such that they are translationally fused to the transcriptional activation domain of GAL4. This library can be co-transformed along with the bait TRAIL gene-GAL4 fusion plasmid into a yeast strain that contains a lacZ gene driven by a promoter that contains GAL4 activation sequence. A cDNA encoded protein, fused to GAL4 transcriptional activation domain, that interacts with bait TRAIL gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait TRAIL gene-interacting protein using techniques routinely practiced in the art.

TRAIL gene products of the invention may, in vivo, interact with one or more macromolecules, including cellular or extracellular macromolecules, such as proteins. Such macromolecules include, but are not limited to, other proteins, such as cellular receptors, or nucleic acid molecules and those proteins identified via methods such as those described, above. For example, the TRAIL gene product can interact with a receptor as a peptide hormone or neuropeptide. For purposes of this discussion, the macromolecules are referred to herein as “binding partners”. Compounds that disrupt TRAIL binding in this way are useful in regulating the activity of the TRAIL gene product, especially mutant TRAIL gene products. For example, such compounds can interfere with the interaction of the TRAIL gene product, with its receptor. Such compounds can include, but are not limited to, molecules such as peptides, and the like, as described, for example, above, which would be capable of gaining access to an TRAIL gene product.

The basic principle of the assay systems used to identify compounds that interfere with the interaction between the TRAIL gene product and its binding partner or partners involves preparing a reaction mixture containing the TRAIL gene product, and the binding partner under conditions and for a time sufficient to allow the two to interact and bind, thus forming a complex. In order to test a compound for inhibitory activity, the reaction mixture is prepared in the presence and absence of the test compound. The test compound can be initially included in the reaction mixture, or may be added at a time subsequent to the addition of the TRAIL gene product and its binding partner. Control reaction mixtures are incubated without the test compound or with a placebo. The formation of any complexes between the TRAIL gene protein and the binding partner is then detected. The formation of a complex in the control reaction, but not in the reaction mixture containing the test compound, indicates that the compound interferes with the interaction of the TRAIL gene protein and the interactive binding partner. Additionally, complex formation within reaction mixtures containing the test compound and normal TRAIL gene protein may also be compared to complex formation within reaction mixtures containing the test compound and a mutant TRAIL gene protein. This comparison may be important in those cases wherein it is desirable to identify compounds that disrupt interactions of mutant but not normal TRAIL gene proteins.

The assay for compounds that interfere with the interaction of the TRAIL gene products and binding partners can be conducted in a heterogeneous or homogeneous format. Heterogeneous assays involve anchoring either the TRAIL gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the TRAIL gene products and the binding partners, e.g., by competition, can be identified by conducting the reaction in the presence of the test substance; i.e., by adding the test substance to the reaction mixture prior to or simultaneously with the TRAIL gene protein and interactive intracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the TRAIL gene product or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the TRAIL gene product or binding partner and drying. Alternatively, an immobilized antibody specific for the species to be anchored may be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the TRAIL gene protein and the interactive binding partner is prepared in which either the TRAIL gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt a TRAIL gene protein/binding partner interaction can be identified.

In a particular embodiment, the TRAIL gene product can be prepared for immobilization using recombinant DNA techniques described above. For example, the TRAIL coding region can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art and described above. This antibody can be labeled with the radioactive isotope 1251, for example, by methods routinely practiced in the art. In a heterogeneous assay, e.g., the GST-TRAIL fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the TRAIL gene protein and the interactive binding partner can be detected by measuring the amount of radioactivity that remains associated with the glutathione-agarose beads. A successful inhibition of the interaction by the test compound will result in a decrease in measured radioactivity.

Alternatively, the GST-TRAIL gene fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the TRAIL gene product/binding partner interaction can be detected by adding the labeled antibody and measuring the radioactivity associated with the beads.

In another embodiment of the invention, these same techniques can be employed using peptide fragments that correspond to the binding domains of the TRAIL protein and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this section, above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the segments can be engineered to express peptide fragments of the protein, which can then be tested for binding activity and purified or synthesized.

For example, and not by way of limitation, a TRAIL gene product can be anchored to a solid material as described, above, in this section by making a GST-TRAIL fusion protein and allowing it to bind to glutathione agarose beads. The interactive binding partner obtained can be labeled with a radioactive isotope, such as 35S, and cleaved with a proteolytic enzyme such as trypsin. Cleavage products can then be added to the anchored GST-TRAIL fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

Compounds including, but not limited to, binding compounds identified via assay techniques such as those described, above, can be tested for the ability to ameliorate symptoms of an apoptosis-related disorder, e.g., apoptosis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; wound granulation; corornary collaterals; cerebral collaterals; arteriovenous malformations; ischemic limb angiogenesis; diabetic neovascularization; macular degeneration; fractures; vasculogenesis; hematopoiesis; ovulation; menstruation; placentation; intestinal adhesions; atherosclerosis; scleroderma; hypertrophic scars, i.e., keloids; cat scratch disease (Rochele minalia quintosa); and ulcers (Helobacter pylori). It TRAIL gene activity by either affecting TRAIL gene expression or by affecting the level of TRAIL gene product activity. For example, compounds can be identified that are involved in another step in the pathway in which the TRAIL gene and/or TRAIL gene product is involved and, by affecting this same pathway may modulate the effect of TRAIL on the development of an apoptosis-related disorder such as cancer. Such compounds can be used as part of a therapeutic method for the treatment of the disorder.

Described below are cell-based and animal model-based assays for the identification of compounds exhibiting such an ability to ameliorate symptoms of an apoptosis-related disorder, e.g., cancer.

First, cell-based systems can be used to identify compounds that can act to ameliorate symptoms of an apoptosis-related disorder, e.g., cancer. Such cell systems can include, for example, recombinant or non-recombinant cell, such as cell lines, that express the TRAIL gene.

In utilizing such cell systems, cells that express TRAIL can be exposed to a compound suspected of exhibiting an ability to ameliorate symptoms of an apoptosis-related disorder, e.g., cancer, at a sufficient concentration and for a sufficient time to elicit such an amelioration of such symptoms in the exposed cells. After exposure, the cells can be assayed to measure alterations in the expression of the TRAIL gene, e.g., by assaying cell lysates for TRAIL mRNA transcripts (e.g., by Northern analysis) or for TRAIL gene products expressed by the cell; compounds that modulate expression of the TRAIL gene are good candidates as therapeutics. Alternatively, the cells are examined to determine whether one or more cellular phenotypes associated with an apoptosis-related disorder, e.g., cancer, has been altered to resemble a more normal or unimpaired, unaffected phenotype, or a phenotype more likely to produce a lower incidence or severity of disorder symptoms.

In addition, animal-based systems or models for an apoptosis-related disorder, e.g., cancer, can be used to identify compounds capable of ameliorating symptoms of the disorder. Such animal-based systems or models can include, for example, transgenic mice, e.g., mice that have been genetically engineered to express exogenous or endogenous TRAIL sequences or, alternatively, to no longer express endogenous TRAIL gene sequences (i.e., “knock-out” mice). Such animal models can be used as test substrates for the identification of drugs, pharmaceuticals, therapies and interventions that can be effective in treating such disorders. For example, animal models can be exposed to a compound suspected of exhibiting an ability to ameliorate symptoms, at a sufficient concentration and for a sufficient time to elicit such an amelioration of symptoms of an apoptosis-related disorder, e.g., cancer, in the exposed animals. The response of the animals to the exposure can be monitored by assessing the reversal of such symptoms.

With regard to intervention, any treatments that reverse any aspect of symptoms of an apoptosis-related disorder, e.g., cancer, should be considered as candidates for human therapeutic intervention in such a disorder. Dosages of test agents may be determined by deriving dose-response curves, as discussed below.

A variety of methods can be employed for the diagnostic and prognostic evaluation of apoptosis-related disorders, such as cancer, and for the identification of subjects having a predisposition to such disorders.

Such methods can, for example, utilize reagents such as the TRAIL gene nucleotide sequences described above, and antibodies directed against TRAIL gene products, including peptide fragments thereof, as described, above. Specifically, such reagents can be used, for example, for:

(1) the detection of the presence of TRAIL gene mutations, or the detection of either over- or under-expression of TRAIL gene mRNA relative to the state of an apoptosis-related disorder, such as cancer;

(2) the detection of either an over- or an under-abundance of TRAIL gene product relative to the unaffected state; and

(3) the detection of an aberrant level of TRAIL gene product activity relative to the unaffected state.

TRAIL gene nucleotide sequences can, for example, be used to diagnose an apoptosis-related disorder using, for example, the techniques for TRAIL mutation detection described above.

The methods described herein can be performed, for example, by utilizing pre-packaged diagnostic kits comprising at least one specific TRAIL gene nucleic acid or anti-TRAIL gene antibody reagent described herein, which may be conveniently used, e.g., in clinical settings, to diagnose subjects exhibiting abnormalities of an apoptosis-related disorder, e.g., cancer.

For the detection of TRAIL mutations, any nucleated cell can be used as a starting source for genomic nucleic acid. For the detection of TRAIL gene expression or TRAIL gene products, any cell type or tissue in which the TRAIL gene is expressed can be utilized.

Nucleic acid-based detection techniques are described, above. Peptide detection techniques are described, above.

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention. Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention also can be used to detect genetic lesions or mutations in a gene of the invention, thereby determining if a subject with the lesioned gene is at risk for a disorder characterized by aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al., 1996, Human Mutation 7:244-255; Kozal et al., 1996, Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. (Examples of sequencing reactions include those based on techniques developed by Maxim and Gilbert, 1977, Proc. Natl. Acad. Sci. USA 74:560 or Sanger, 1977, Proc. Natl. Acad. Sci. USA 74:5463). It is also contemplated that any of a variety of automated sequencing procedures can be utilized when performing the diagnostic assays (1995, Bio/Techniques 19:448), including sequencing by mass spectrometry (see, e.g., PCT Publication No. WO 94/16101; Cohen et al., 1996, Adv. Chromatogr. 36:127-162; and Griffin et al., 1993, Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al., 1985, Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. (See, e.g., Cotton et al., 1988, Proc. Natl. Acad. Sci. USA 85:4397; Saleeba et al., 1992, Methods Enzymol. 217:286-295.) In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair enzymes”) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al., 1994, Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. (See, e.g., U.S. Pat. No.5,459,039.)

In other embodiments, alterations in electrophoretic mobility will be used to identify mutations in genes. For example, SSCP may be used to detect differences in electrophoretic mobility between mutant and wild type nucleic acids (Orita et al., 1989, Proc. Natl. Acad. Sci. USA 86:2766; see also Cotton, 1993, Mutat. Res. 285:125-144; Hayashi, 1992, Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., 1991, Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al., 1985, Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, 1987, Biophys. Chem. 265:12753).

Examples of other techniques for detecting point mutations include, but are not limited to, selective oligonucleotide hybridization, selective amplification, or selective primer extension. For example, oligonucleotide primers can be prepared in which the known mutation is placed centrally and then hybridized to target DNA under conditions which permit hybridization only if a perfect match is found (Saiki et al., 1986, Nature 324:163; Saiki et al., 1989, Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification may carry the mutation of interest in the center of the molecule (so that amplification depends on differential hybridization) (Gibbs et al., 1989, Nucleic Acids Res. 17:2437-2448) or at the extreme 3′ end of one primer where, under appropriate conditions, mismatch can prevent or reduce polymerase extension (Prossner, 1993, Tibtech 11:238). In addition, it may be desirable to introduce a novel restriction site in the region of the mutation to create cleavage-based detection (Gasparini et al., 1992, Mol. Cell Probes 6:1). It is anticipated that in certain embodiments amplification can also be performed using Taq ligase for amplification (Barany, 1991, Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which may be conveniently used, e.g., in clinical settings to diagnose subjects exhibiting symptoms or family history of a disease or illness involving a gene encoding a polypeptide of the invention. Furthermore, any cell type or tissue, preferably peripheral blood leukocytes, in which the polypeptide of the invention is expressed, can be utilized in the prognostic assays described herein.

The invention further provides kits that facilitate the use and/or detection of TRAIL genes and gene products described herein. The kits described herein can be conveniently used, e.g., in clinical settings to diagnose subjects exhibiting symptoms or family history of a disease or illness involving a gene encoding a polypeptide of the invention. Furthermore, any cell type or tissue in which the polypeptide of the invention is expressed can be utilized in the prognostic assays described herein.

In one embodiment, a diagnostic test kit for identifying cells or tissues which mis-express TRAIL genes or gene products is provided. In this embodiment, a diagnostic kit is provided, with one or more containers comprising an oligonucleotide, e.g., a detectably labeled oligonucleotide, which hybridizes to a nucleic acid sequence encoding a polypeptide of the invention. In another embodiment, a kit is provided, with one or more containers comprising a pair of primers useful for amplifying a nucleic acid molecule encoding a polypeptide of the invention. In various other embodiments, the kit can also comprise, e.g., a buffering agent, a preservative, or a protein stabilizing agent. The kit also can comprise components necessary for detecting the detectable agent (e.g., an enzyme or a substrate). The kit also can contain a control sample or a series of control samples which can be assayed and compared to the test sample. Each component of the kit is usually enclosed within an individual container and all of the various containers are within a single package along with instructions for observing whether the tested subject is suffering from, or is at risk of developing, a disorder associated with aberrant expression of the polypeptide. Such a kit can be used, for example, to measure the levels of a nucleic acid molecule encoding the protein in a sample of cells from a subject, e.g., detecting mRNA levels or determining whether a gene encoding the protein has been mutated or deleted.

In another embodiment, the invention provides kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention as discussed, for example, in sections above relating to uses of the sequences of the invention. In this embodiment, a kit is provided, with one or more containers comprising: (1) a first antibody (e.g., attached to a solid support) which binds to a polypeptide of the invention; and, optionally, (2) a second, different antibody which binds to either the polypeptide or the first antibody and is conjugated to a detectable agent. Such kits can be used to determine if a subject is suffering from, or is at increased risk of, an apoptosis-related disorder, such as cancer.

Described below are methods and compositions whereby a TRAIL-mediated process can be modulated and/or whereby an apoptosis-related disorder, e.g., cancer, can be treated.

For example, such methods can comprise administering compounds which modulate the expression of a TRAIL gene and/or the synthesis or activity of a TRAIL gene product so that the process is modulated or a symptom of the disorder is ameliorated.

Alternatively, in those instances whereby the apoptosis-related disorder, e.g., cancer, results from TRAIL gene mutations that lower or abolish TRAIL activity, respectively, such methods can comprise supplying a mammal with a nucleic acid molecule encoding an unimpaired TRAIL gene product such that an unimpaired TRAIL gene product is expressed and symptoms of the disorder are ameliorated.

In another embodiment of methods for the treatment of mammalian apoptosis-related disorder, e.g., cancer, resulting from TRAIL gene mutations, such methods can comprise supplying a mammal with a cell comprising a nucleic acid molecule that encodes an unimpaired TRAIL gene product such that the cell expresses the unimpaired TRAIL gene product and symptoms of the disorder are ameliorated.

In cases in which a loss of normal TRAIL gene product function results in the development of an apoptosis-related disorder phenotype, e.g., cancer, an increase in TRAIL gene product activity would facilitate progress towards an asymptomatic state in individual organisms exhibiting a deficient level of TRAIL gene expression and/or TRAIL gene product activity. Methods for enhancing the expression or synthesis of TRAIL can include, for example, methods such as those described below.

Alternatively, symptoms of apoptosis-related disorder phenotype, e.g., cancer, may be ameliorated by administering a compound that decreases the level of TRAIL gene expression and/or TRAIL gene product activity. Methods for inhibiting or reducing the level of TRAIL synthesis or expression can include, for example, methods such as those described below.

In one embodiment of treatment methods, the compounds administered do not comprise compounds, in particular drugs, reported to ameliorate or exacerbate the symptoms of an angiogenesis-related disorder. If such treatment methods do comprise such compounds, preferably such compounds are utilized in a manner (e.g., different dosage, mode of administration, and/or co-administration with one or more additional compounds) that differs from the manner in which such compounds have been administered previously.

In another embodiment, symptoms of a disorder described herein, e.g., cancer, can be ameliorated by TRAIL protein therapy methods, e.g., decreasing or increasing the level and/or activity of TRAIL using the TRAIL protein, fusion protein, and peptide sequences described above, or by the administration of proteins or protein fragments (e.g., peptides) which interact with a TRAIL gene or gene product and thereby inhibit or potentiate its activity.

Such protein therapy can include, for example, the administration of a functional TRAIL protein or fragments of a TRAIL protein (e.g., peptides) which represent functional TRAIL domains.

In one embodiment, TRAIL fragments or peptides representing a functional TRAIL binding domain are administered to an individual organism such that the peptides bind to a TRAIL binding protein, e.g., a TRAIL receptor. Such fragments or peptides can serve to inhibit TRAIL activity in an individual organism by competing with, and thereby inhibiting, binding of TRAIL to the binding protein, thereby ameliorating symptoms of a disorder described herein. Alternatively, such fragments or peptides can enhance TRAIL activity in an individual organism by mimicking the function of TRAIL in vivo, thereby ameliorating the symptoms of a disorder described herein.

The proteins and peptides which can be used in the methods of the invention include synthetic (e.g., recombinant or chemically synthesized) proteins and peptides, as well as naturally occurring proteins and peptides. The proteins and peptides can have both naturally occurring and non-naturally occurring amino acid residues (e.g., D-amino acid residues) and/or one or more non-peptide bonds (e.g., imino, ester, hydrazide, semicarbazide, and azo bonds). The proteins or peptides can also contain additional chemical groups (i.e., functional groups) present at the amino and/or carboxy termini, such that, for example, the stability, bioavailability, and/or inhibitory activity of the peptide is enhanced. Exemplary functional groups include hydrophobic groups (e.g. carbobenzoxyl, dansyl, and t-butyloxycarbonyl, groups), an acetyl group, a 9-fluorenylmethoxy-carbonyl group, and macromolecular carrier groups (e.g., lipid-fatty acid conjugates, polyethylene glycol, or carbohydrates) including peptide groups.

In another embodiment, symptoms of certain apoptosis-related disorders, such as cancer, can be ameliorated by decreasing the level of TRAIL gene expression and/or TRAIL gene product activity by using TRAIL gene sequences in conjunction with well-known antisense, gene “knock-out,” ribozyme and/or triple helix methods to decrease the level of TRAIL gene expression. Among the compounds that can exhibit the ability to modulate the activity, expression or synthesis of the TRAIL gene, including the ability to ameliorate the symptoms of an angiogenesis-related disorder, e.g., cancer, are antisense, ribozyme, and triple helix molecules. Such molecules can be designed to reduce or inhibit either unimpaired, or if appropriate, mutant target gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA can thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the TRAIL gene can be used in an antisense approach to inhibit translation of endogenous TRAIL mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86, 6553-6556; Lemaitre, et al., 1987, Proc. Natl. Acad. Sci. 84, 648-652; PCT Publication No. W088/09810, published December 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6, 958-976) or intercalating agents (see, e.g., Zon, 1988, Pharm. Res. 5, 539-549). To this end, the oligonucleotide can be conjugated to another molecule, e.g., a peptide, hybridization triggered cross-linking agent, transport agent, hybridization-triggered cleavage agent, etc.

The antisense oligonucleotide can comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide can also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier, et al., 1987, Nucl. Acids Res. 15, 6625-6641). The oligonucleotide is a 2′-0-methylribonucleotide (Inoue, et al., 1987, Nucl. Acids Res. 15, 6131-6148), or a chimeric RNA-DNA analogue (Inoue, et al., 1987, FEBS Lett. 215, 327-330).

Oligonucleotides of the invention can be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence can be used, those complementary to the transcribed, untranslated region are most preferred.

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the subject will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Benoist and Chambon, 1981, Nature 290, 304-310), the promoter contained in the 3′ long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences can be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity can be introduced into cells via gene therapy methods such as those described, below, that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention can be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

With respect to an increase in the level of normal TRAIL gene expression and/or TRAIL gene product activity, TRAIL gene nucleic acid sequences, described above, for example, can be utilized for the treatment of an angiogenesis-related disorder, e.g., cancer. Such treatment can be administered, for example, in the form of gene replacement therapy. Specifically, one or more copies of a normal TRAIL gene or a portion of the TRAIL gene that directs the production of a TRAIL gene product exhibiting normal TRAIL gene function, may be inserted into the appropriate cells within a subject, using vectors that include, but are not limited to, adenovirus, adeno-associated virus, herpesvirus and retrovirus vectors, in addition to other particles that introduce DNA into cells, such as liposomes.

Because TRAIL genes can be expressed in the brain, such gene replacement therapy techniques should be capable delivering TRAIL gene sequences to these cell types within subjects. Thus, in one embodiment, techniques that are well known to those of skill in the art (see, e.g., PCT Publication No. WO89/10134, published Apr. 25, 1988) can be used to enable TRAIL gene sequences to cross the blood-brain barrier readily and to deliver the sequences to cells in the brain. With respect to delivery that is capable of crossing the blood-brain barrier, viral vectors such as, for example, those described above, are preferable.

In another embodiment, techniques for delivery involve direct administration of such TRAIL gene sequences to the site of the cells in which the TRAIL gene sequences are to be expressed.

Additional methods that can be utilized to increase the overall level of TRAIL gene expression and/or TRAIL gene product activity include the introduction of appropriate TRAIL-expressing cells, preferably autologous cells, into a subject at positions and in numbers that are sufficient to ameliorate the symptoms of an apoptosis-related disorder, e.g., cancer. Such cells may be either recombinant or non-recombinant.

Among the cells that can be administered to increase the overall level of TRAIL gene expression in a subject are normal cells, preferably liver cells, that express the TRAIL gene.

Alternatively, cells, preferably autologous cells, can be engineered to express TRAIL gene sequences, and may then be introduced into a subject in positions appropriate for the amelioration of the symptoms of an apoptosis-related disorder, e.g., cancer. Alternately, cells that express an unimpaired TRAIL gene and that are from an MHC matched individual organism can be utilized, and may include, for example, liver cells. The expression of the TRAIL gene sequences is controlled by the appropriate gene regulatory sequences to allow such expression in the necessary cell types. Such gene regulatory sequences are well known to the skilled artisan. Such cell-based gene therapy techniques are well known to those skilled in the art, see, e.g., Anderson, U.S. Pat. No. 5,399,349.

When the cells to be administered are non-autologous cells, they can be administered using the well known techniques that prevent a host immune response against the introduced cells from developing. For example, the cells can be introduced in an encapsulated form which, while allowing for an exchange of components with the immediate extracellular environment, does not allow the introduced cells to be recognized by the host immune system.

Additionally, compounds, such as those identified via techniques such as those described above, that are capable of modulating TRAIL gene product activity can be administered using standard techniques that are well known to those of skill in the art. In instances in which the compounds to be administered are to involve an interaction with brain cells, the administration techniques should include the well known ones that allow for a crossing of the blood-brain barrier.

Agents, or modulators, which have a stimulatory or inhibitory effect on activity or expression of a polypeptide of the invention as identified by a screening assay described herein can be administered to individual organisms to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual organism may be considered. Differences in metabolism of therapeutics can lead to severe toxicity or therapeutic failure by altering the relation between dose and blood concentration of the pharmacologically active drug. Thus, the pharmacogenomics of the individual organism permits the selection of effective agents (e.g., drugs) for prophylactic or therapeutic treatments based on a consideration of the individual organism's genotype. Such pharmacogenomics can further be used to determine appropriate dosages and therapeutic regimens. Accordingly, the activity of a polypeptide of the invention, expression of a nucleic acid of the invention, or mutation content of a gene of the invention in an individual organism can be determined to thereby select appropriate agent(s) for therapeutic or prophylactic treatment of the individual organism.

Pharmacogenomics deals with clinically significant hereditary variations in the response to drugs due to altered drug disposition and abnormal action in affected individual organisms. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extremes are the so-called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual organism can be determined to thereby select appropriate agent(s) for therapeutic or prophylactic treatment of the individual organism. In addition, pharmacogenetic studies can be used to apply genotyping of polymorphic alleles encoding drug-metabolizing enzymes to the identification of an individual organism's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

Monitoring the influence of agents (e.g., drugs, compounds) on the expression or activity of a polypeptide of the invention (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual organism with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the preadministration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to increase the expression or activity of the polypeptide to higher levels than detected, i.e., to increase the effectiveness of the agent. Alternatively, decreased administration of the agent may be desirable to decrease expression or activity of the polypeptide to lower levels than detected, i.e., to decrease the effectiveness of the agent.

The compounds of this invention can be formulated and administered to inhibit a variety of angiogenesis-related disorders by any means that produces contact of the active ingredient with the agent's site of action in the body of a mammal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The dosage administered will be a therapeutically effective amount of the compound sufficient to result in amelioration of symptoms of the angiogenesis-related disorder and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Specific dosages may also be utilized for antibodies. Typically, the preferred dosage is 0.1 mg/kg to 100 mg/kg of body weight (generally 10 mg/kg to 20 mg/kg), and if the antibody is to act in the brain, a dosage of 50 mg/kg to 100 mg/kg is usually appropriate. If the antibody is partially human or fully human, it generally will have a longer half-life within the human body than other antibodies. Accordingly, lower dosages of partially human and fully human antibodies is often possible. Additional modifications may be used to further stabilize antibodies. For example, lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

A therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

Moreover, treatment of a subject with a therapeutically effective amount of a protein, polypeptide or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5 or 6 weeks.

The present invention further encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors known to those or ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about I microgram per kilogram to about 500 milligrams per kilogram, about 100 micrograms per kilogram to about 5 milligrams per kilogram, or about I microgram per kilogram to about 50 micrograms per kilogram.

Pharmaceutical compositions for use in accordance with the present invention can be formulated in conventional manner using one or more physiologically acceptable carriers or excipients.

Thus, the compounds and their physiologically acceptable salts and solvates can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g. gelatin, for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient can be in powder form for constitution with a suitable vehicle, e.g., sterile pyrogen-free water, before use. In general, water, a suitable oil, saline, aqueous dextrose (glucose), and related sugar solutions and glycols such as propylene glycol or polyethylene glycols are suitable carriers for parenteral solutions. Solutions for parenteral administration contain preferably a water soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations may be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release.

Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

The compositions can, if desired, be presented in a pack or dispenser device which can contain one or more unit dosage forms containing the active ingredient. The pack can for example comprise metal or plastic foil, such as a blister pack. The pack or dispenser device may be accompanied by instructions for administration.

Useful pharmaceutical dosage forms, for administration of the compounds of this invention can be illustrated as follows:

Capsules: Capsules are prepared by filling standard two-piece hard gelatin capsulates each with the desired amount of powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing the desired amount of the active ingredient. The capsules are then washed and dried.

Tablets: Tablets are prepared by conventional procedures so that the dosage unit is the desired amount of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings can be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 millimeters contain 100 milligrams of finely divided active ingredient, 200 milligrams of sodium carboxymethyl cellulose, 5 milligrams of sodium benzoate, 1.0 grams of sorbitol solution U.S.P. and 0.025 millimeters of vanillin.

Gene Therapy Administration: Where appropriate, the gene therapy vectors can be formulated into preparations in solid, semisolid, liquid or gaseous forms such as tablets, capsules, powders, granules, ointments, solutions, suppositories, injections, inhalants, and aerosols, in the usual ways for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds.

Accordingly, the pharmaceutical composition of the present invention can be delivered via various routes and to various sites in an animal body to achieve a particular effect (see, e.g., Rosenfeld et al. (1991), supra; Rosenfeld et al., Clin. Res., 3 9(2), 31 1A (1991 a); Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular; intravenous, peritoneal, subcutaneous, intradermal, as well as topical administration.

The compositions of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and animal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

Accordingly, the present invention also provides a method of transferring a therapeutic gene to a host, which comprises administering the vector of the present invention, preferably as part of a composition, using any of the aforementioned routes of administration or alternative routes known to those skilled in the art and appropriate for a particular application. The “effective amount” of the composition is such as to produce the desired effect in a host which can be monitored using several end-points known to those skilled in the art. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on individual differences in pharmacokinetics, drug disposition, and metabolism. Similarly, amounts can vary in in vitro applications depending on the particular cell line utilized (e.g., based on the number of adenoviral receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

The original clones in connection with the present invention isolated by using the Invitrogen kit were deposited on Apr. 15, 2003 with the American Type Culture Collection, 10801 University Boulevard, Manassas, Va. 20110-2209. These clones are E. coli PIR1 strain INV-1 containing plasmid p11caTRAIL (ATCC Deposit Number PTA-5137), E. coli PIR1 strain INV-2 containing plasmid p14feTRAIL (ATCC Deposit Number PTA-5138), E. coli PIR1 strain INV-3 containing plasmid p68caTRAILsh (ATCC Deposit Number PTA-5139) and E. coli PIR1 strain INV-4 containing plasmid p8feTRAILsh (ATCC Deposit Number PTA-5140). TRAILsh is the soluble form of TRAIL.

The following examples are offered by way of example, and are not intended to limit the scope of the invention in any manner.

### EXAMPLE 1

### Identification and Cloning of Trail Genes

In the Example presented in this section, studies are described that identify novel canine genes, referred to herein as TRAIL, which are involved in apoptosis-related disorders, e.g., cancer.

1. The isolation of RNA from canine and feline cell lines. Three cell lines were obtained from ATCC (American Type Culture Collection, Rockville, Md.): CRL-6130 (Fc28.Lu, feline lung), CRL-6252 (ECF50.HT, canine heart), CRL-6569 ((Fc2.Lu, feline lung). T75 flasks of confluent cells of above three cell lines were lysed and total RNA was purified using Sv Total RNA Isolation System following instructions from manufacturer (Promega Inc., Calif.). 10-45 ug of RNA was isolated and was suspended in 100 ul of H₂0.

2. RT-PCR amplification and detection of TRAIL in those cell lines. Two pairs of primers were designed to amplify internal regions of canine and feline TRAIL cDNA based on consensus sequences from human (accession number U37518) and mouse (accession number U37522). The 5′ primer of both sets: N4066E09-ACCATTTCTACAGTTCMAGAAAAGCA (SEQ ID NO: 1) corresponding to nucleotide # 382-408 of human TRAIL cDNA as well as nucleotide # 354-380 of mouse TRAIL cDNA. The 3′ primer of set 1: N4066E06-TCCTGAAATCGRAAGTATGTTTGGGAATACATGTA (SEQ ID NO: 2), corresponding to nucleotide # 634-669 of human TRAIL cDNA. The 3′ primer of set 2: N4066E1 1-ACAGAAACAAAAATYCTGTCATTTT (SEG ID NO: 3), corresponding to nucleotide #841-866 of human TRAIL cDNA. Those oligo primers were used for the amplification reaction. The RT-PCR reaction was performed using Ready-To-Go RT-PCR Kit from Amersham Pharmacia (cat no. 27-9267-01, Illinois). 1 ug of total RNA was added to the RT-PCR beads together with 1 ug of pd(N)₆ as the first strand primer. The reverse transcription reaction was performed at 42° C. for 30 minutes. The reaction was then incubated at 95° C. for 5 minutes to inactivate the reverse transcriptase and to completely denature the template. The PCR program was as follows: hold at 95° C. for 5 minutes, followed by 30 cycles of 30 seconds at 94° C., 1 minute at 52° C., 1 minutes at 68° C. and 7 minutes of extension at 74° C. The PCR products were analyzed by electrophoresis.

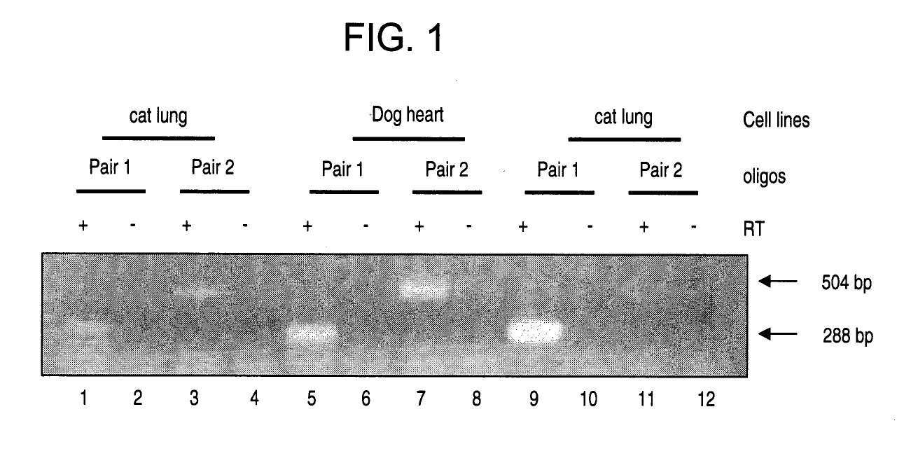

Total RNA was isolated from three canine and feline cell lines (CRL-6130, Fc28.Lu, feline lung; CRL-6252 ECF50.HT canine heart; CRL-6569 Fc2.Lu, feline lung). In order to test the gene expression of TRAIL in those cell lines, two pairs of primers amplifying internal regions of canine and feline TRAIL were used for the RT-PCR reactions. One pair of primers generated a 504 bp PCR fragment, while the other generated a 288 bp fragment. The result clearly showed the abundant expression of TRAIL in these three cell lines (FIG. 1).

3. RACE-PCR of canine and feline TRAIL fragments. Three internal TRAIL oligo primers were successfully used in PCR-RACE. They are: N4066E06-TCCTGAAATCGRAAGTATGTTTGGGAATACATGTA (SEQ ID NO: 2, see above), N4066E07-TACATCTATTCCCAAACATACTTYCGATTTCAGGA (SEQ ID NO: 4), corresponding to nucleotide # 634-669 of human TRAIL cDNA as well as nucleotide # 605-640 of mouse TRAIL cDNA. N4066E09-ACCATTTCTACAGTTCMAGAAAAGCA (SEQ ID NO: 1, see above). The PCR-RACE reaction was performed using SMART RACE cDNA Amplification Kit from Clontech Laboratories (cat no. K1811-1, Palo Alto, Calif.). The first-strand cDNA synthesis was carried out to prepare 5′-RACE-Ready cDNA and 3′-RACE Ready cDNA, respectively. 1 ug of total canine and feline RNA (see above) was mixed together with 5′-CDS primer as well as SMART II oligo for 5′-RACE-Ready DNA. For 3′-RACE Ready cDNA, RNA samples were mixed with 3′-CDS primer. The reactions were incubated at 70° C. for 2 minutes and then cool on ice for 2 minutes. First-Strand buffer, DTT, dNTP mix and MMLV reverse transcriptase were added to the mixture and incubated at 42° C. for 1.5 hours. 100 ul of Tricine-EDTA buffer was then added and stored at −20° C.

RACE (Rapid Amplification of cDNA Ends) was carried out according to CLONTECH's instruction manual. PCR master mix was prepared by mixing water with Advantage 2 PCR buffer, dNTP mix and Advantage 2 polymerase mix. 5′ RACE sample was obtained by mixing 5′-RACE-Ready cDNA (see above), 10×UPM, gene-specific primer (primers E07 or E09, see above) and PCR master mix. 3′ RACE sample was obtained by mixing 3′-RACE-Ready cDNA (see above), 10×UPM, gene-specific primer (primers E06, see above) and PCR master mix. The Touch Down PCR program was as follows: 5 cycles of 5 seconds at 94° C. and 3 minutes at 72° C., followed by 5 cycles of 5 seconds at 94° C. and 10 seconds at 70° C. and 3 minutes at 72° C., followed again by 35 cycles of 5 seconds at 94° C., 10 seconds at 65° C. and 3 minutes at 72° C. The PCR products were analyzed by electrophoresis. Possible RACE DNA bands were further analyzed by Southern analysis. The transfer was carried out with Turboblotter of Schleicher & Schuell Inc. Human TRAIL DNA probe was made with DIG kit of Boeringer Mannheim Inc.

4. 1 ul of human spleen marathon-ready cDNA (Clonetech Inc.) was mixed together with primers N4066E12 (5′-GCAGTCAGACTCTGACAGGATCATG, SEQ ID NO: 5) and N4066F02 (5′-CTTTTTCTTTCCAGGTCAGTTA, SEQ ID NO: 6), PCR buffer, DIG mix and enzyme mix. The PCR was carried out as follows: hold at 94° C. for 3 minutes, followed by 30 cycles of 30 seconds at 94° C., 1 minute at 55° C., 1.5 minutes at 68° C. and 7 minutes of extension at 74° C. The DIG-labeled PCR products were analyzed by electrophoresis.

Pre-hybridization was carried out at 42° C. for 3 hours. Hybridization with DIG-labeled TRAIL PCR DNA was carried out overnight at 42° C. The blot was washed twice in buffer containing 2×SSC and 0.1% SDS for 5 minutes, followed by washing twice in buffer containing 0.1×SSC and 0.1% SDS at 65° C.

ECL detection was performed by incubating the membrane in 100 ml blocking solution for 30 minutes, followed by incubating for 30 minutes in 20 ml antibody solution (anti-DIG-AP, 75 mU/ml). Then the blot was washed twice in washing buffer (Maleic acid buffer plus 0.3% Tween 20, v/v) for 15 minutes. After equilibrating the membrane in 20 ml detection buffer for 2-5 minutes, CDP-Star (at 1:100 dilution) was added to the membrane and exposed to X-ray film.

5. Cloning of TRAIL RACE DNA. The RACE PCR products were cloned into Eukaryotic TA cloning vector pUni/V5-His-TOPO (Invitrogen, Carlsbad, Calif.) following manufacturer's instructions and designated as pUni-caTrail-E06, pUni-caTrail-E07 and pUni-caTrail-E09, respectively for canine clones. For feline clones, they are designated as pUni-feTrail-E06, pUni-feTrail-E07 and pUni-feTrail-E09, respectively. Four independent clones containing inserts of the expected size (0.7 kb for RACE primer E06, 1.1 kb for RACE primer E07 and 0.7 kb for RACE primer E09) were sequenced (Advanced Genetic Analysis Center, St. Paul, Minn.). The sequences were assembled and analyzed using DNAStar (DNAStar Inc. Madison, Wis.).

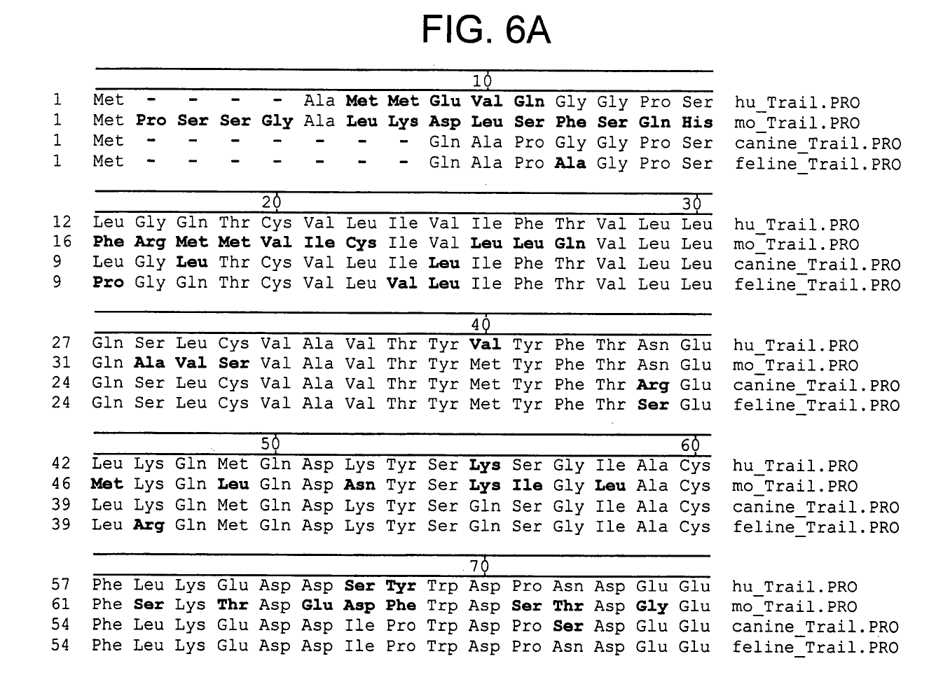

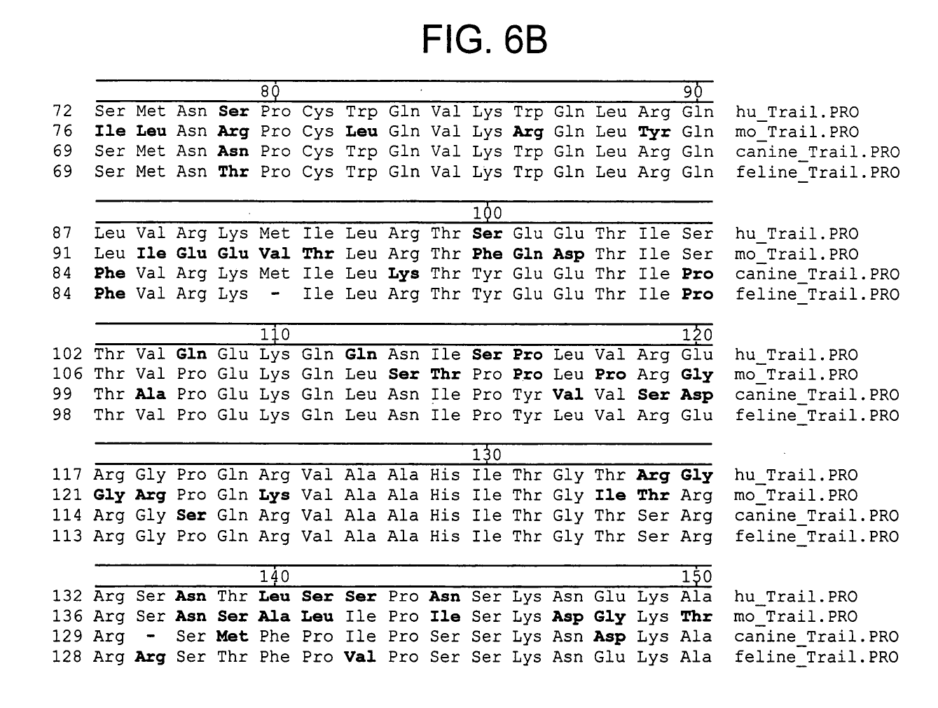

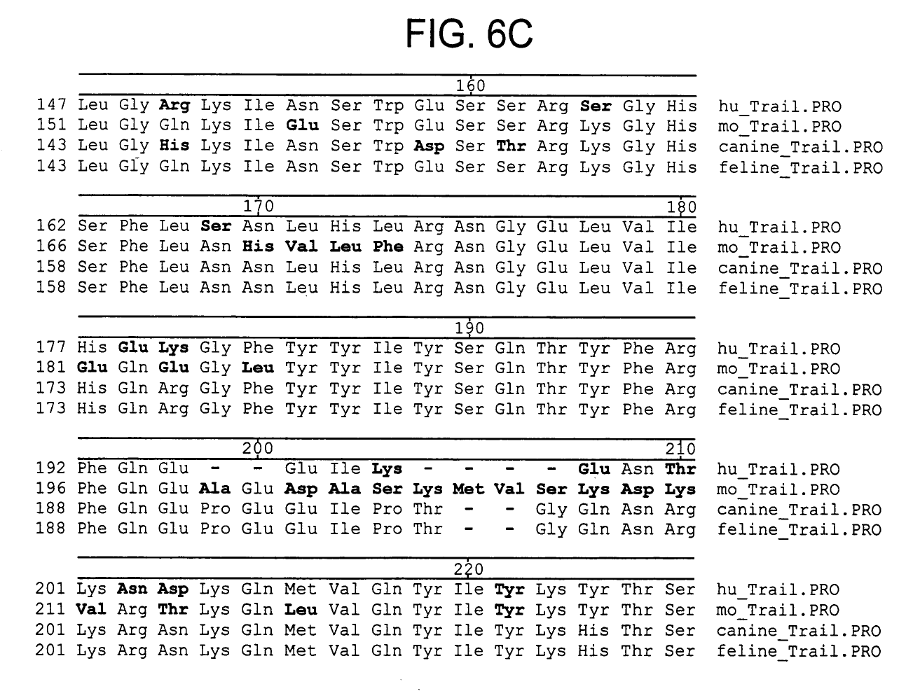

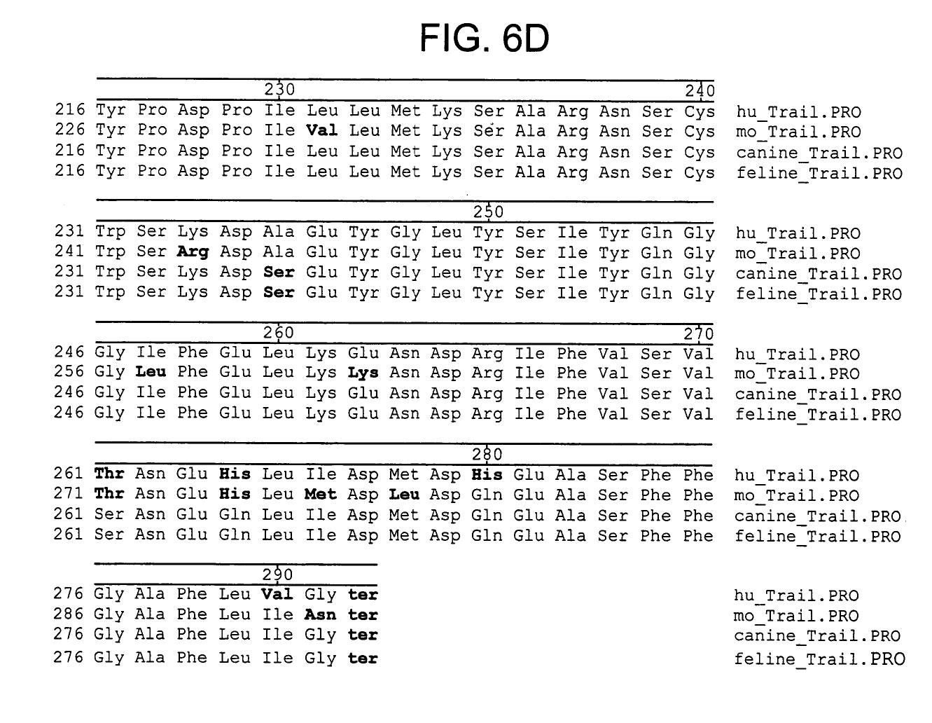

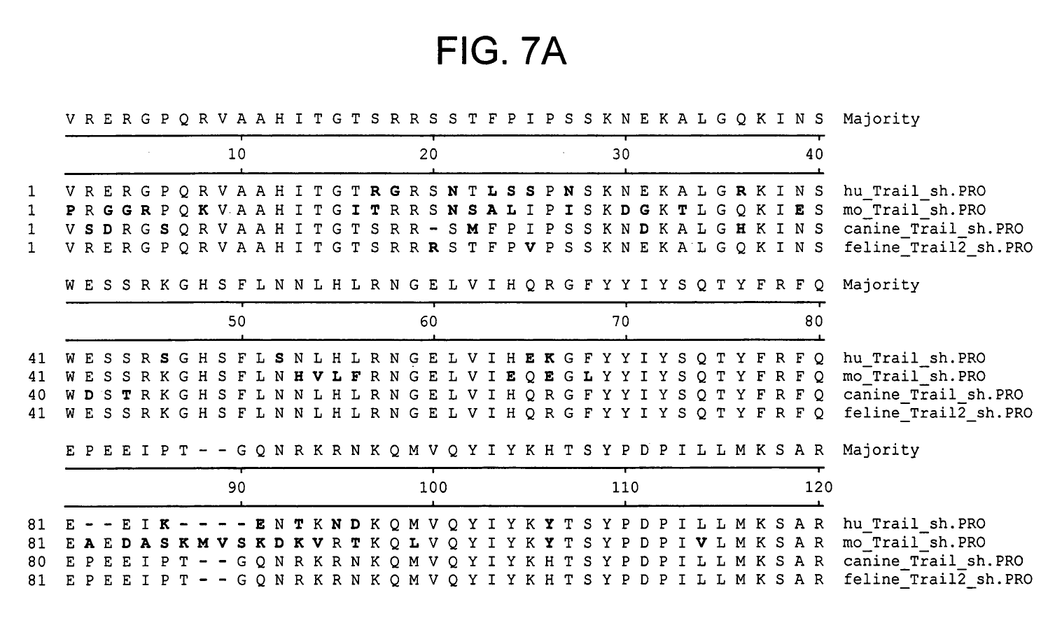

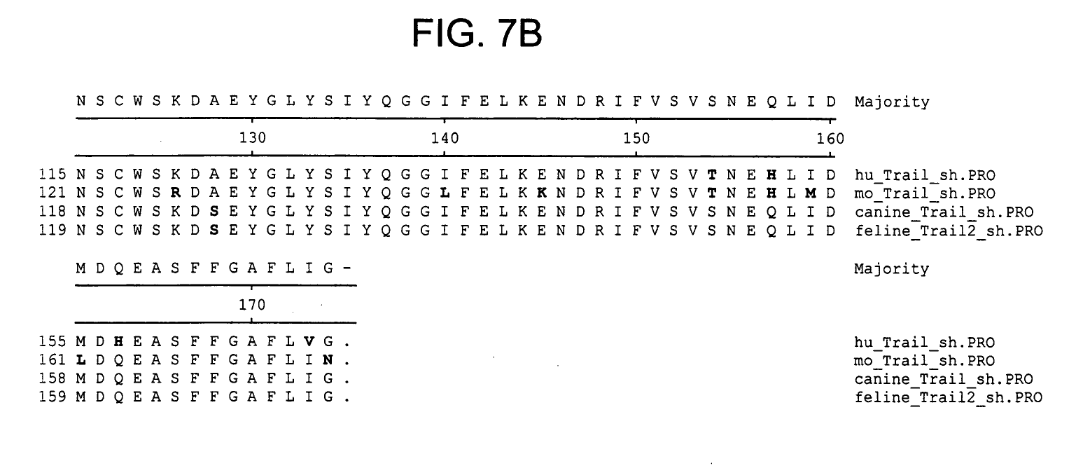

Due to the lack of DNA sequence homology between human and murine TRAIL, internal primers were designed to use RACE technology to obtain the canine and feline cDNA. The cDNAs encoding a portion of canine and feline TRAIL were amplified by RACE-PCR from canine and feline. The nucleotide sequence of canine TRAIL is shown in FIG. 2 (SEQ ID NO: 20), and the predicted amino acid sequence is shown in FIG. 3 (SEQ ID NO: 21). The nucleotide sequence of feline TRAIL is shown in FIG. 4 (SEQ ID NO: 22), and the predicted amino acid sequence is shown in FIG. 5 (SEQ ID NO: 23). The region corresponding to the open reading frame coding for canine TRAIL protein is in bold in FIG. 2 and the region corresponding to the coding sequence of feline TRAIL protein is in bold in FIG. 4. FIGS. 6A-6D shows the alignment of all known amino acid sequences of TRAIL, and the degree of homology between canine TRAIL and that of human and mouse is 80.3% and 63.5% respectively. The degree of homology between feline TRAIL and that of human and mouse is 83.2% and 65.3% respectively. The degree of homology between canine and feline TRAIL is 92.9%. FIGS. 7A-7B shows the alignment of all known amino acid sequences of soluble TRAIL, and the degree of homology between canine soluble TRAIL and that of human and mouse is 76.9% and 66.9% respectively. The degree of homology between feline soluble TRAIL and that of human and mouse is 82.2% and 69.9% respectively. The degree of homology between soluble canine and feline TRAIL is 92.4%.

6. Cloning of full-length canine and feline TRAIL. The full-length canine and feline genes were obtained as follows. For feline TRAIL, 5′-RACE-Ready DNA (see above) and 3′-RACE-Ready DNA were used as templates for RCR reactions, respectively. The primers used are: N7974C01-AGAGTACGCGGGGGCAGCAGTGAC (SEQ ID NO: 7) for upstream primer and N7974C02-CCCTCGAGTGTAGCCGATTAAAAAGGCCCCGAAAAAAC (SEQ ID NO: 8) for downstream primer. The PCR program used was as follows: 5 minutes at 95° C. to denature the template, followed by 30 cycles of 30 seconds at 94° C. and 1 minute at 55° C. and 1.5 minutes at 68° C., followed by 7 minutes at 74° C. The PCR products were analyzed by electrophoresis. Possible full-length TRAIL DNA was further analyzed by Southern analysis as described above. DIG-labeled Human TRAIL DNA as used as probe.

The PCR products which hybridized TRAIL probe were again analyzed by electrophoresis and DNA bands of approximately 0.9 kb length were excised and isolated from agarose gel using QIAquick Gel Extraction Kit. The purified band was then cloned into pUni/V5-His-TOPO cloning vector (Invitrogen, Carlsbad, Calif.) following manufacturer's instructions. Twenty-two independent clones containing inserts of the expected size (0.9 kb) were sequenced (Advanced Genetic Analysis Center, St. Paul, Minn.). Only one clone (#14) had the complete correct sequence when compared with feline RACE sequence. It was designated as pUni-feTRAIL-#14.

For canine TRAIL, dog heart total RNA (CRL 6252) was used for the first-strand cDNA synthesis reaction in the presence of oligo pdN6 and AMV reverse transcriptase. The reaction was performed at room temperature for 10 minutes, then at 42° C. for 50 minutes followed by at 70° C. for 10 minutes. The above DNA was then used as templates for PCR reactions. The primers used are: N7429A12-GCAGTGGATCCAACGCAGAGTACGCGGGAGCACGGACCGGCGGGGGGCAG (SEQ ID NO: 9) for upstream primer and N9294G07-CCAAGAGTAGATAATAAAGACAGC (SEQ ID NO: 10) for down stream primer. Pfx polymerase (GIBCO-Life Technologies) was used in the PCR reaction. The PCR program used was as follows: 5 minutes at 95° C. to denature the template, followed by 30 cycles of 30 seconds at 94° C. and 1 minute at 55° C. and 1.5 minutes at 68° C., followed by 7 minutes at 74° C. The PCR products were analyzed by electrophoresis. An abundant band of about 1.2 kb was clearly visible. This band was further confirmed by Southern analysis using DIG-labeled Human TRAIL DNA as a probe.

The above PCR product was then cloned into pUni/V5-His-TOPO cloning vector (Invitrogen, Carlsbad, Calif.) following manufacturer's instructions. Twenty independent clones containing inserts of the expected size (1.2 kb) were sequenced (Advanced Genetic Analysis Center, St. Paul, Minn.). Fourteen out of those twenty clones (#1, 2, 3, 4, 5, 8, 10, 11, 14, 17, 18, 22, 24, and 25) had a complete correct sequence when compared with the canine RACE sequence. It was designated as pUni-caTRAIL-#1, etc.

Therefore, full-length canine and feline cDNA clones were obtained by PCR reactions using first-strand cDNA as template. For canine TRAIL, dog heart total RNA (CRL 6252) was used for the first-strand cDNA synthesis reaction in the presence of oligo pdN6 and AMV reverse transcriptase. For feline TRAIL, 5′-RACE-Ready DNA (cat lung, see above) and 3′-RACE-Ready DNA were used as templates for RCR reactions, respectively. The primers used locate just outside of the TRAIL coding region. Both canine and feline full-length clones were analyzed by restriction analysis and confirmed by sequence analysis.

7. Cloning of soluble canine and feline TRAIL. In this experiment, human soluble TRAIL gene was obtained as a positive control. Human spleen marathon-ready DNA was used in PCR reaction using Advantage HF (high fidelity DNA polymerase, Invitrogen Inc.). The upstream primer for human soluble TRAIL used: N7974C06-CAACAAAATATGGATCCCATGGTGAGAGAAAGAGGT (SEQ ID NO: 11). The downstream primer which created fusion with V5 tag and His tag in the vector: R4428C10-TTCCAGGCTCGAGAGCCAACTAAAAAGGCCCCGAAAAAAC (SEQ ID NO: 12). The downstream primer without creating V5-His tag, R4428C11-TTCTCGAGCAGTTAGCCAACTAAAAAGGCCCCGAAAAAAC (SEQ ID NO: 13). The upstream primer for canine soluble TRAIL: N8071H11-ATTCCTTACATGGTAAGCGACCGAGGTTCTCAGAG (SEQ ID NO: 14). The down stream primer which created fusion with V5 tag and His tag in the vector: R1886B08-GTTTTTTCTCGAGTGCAGCGTATGTAGCCGATTAAA (SEQ ID NO: 15). The downstream primer without creating V5-His tag, R4429A09-GTTTTTTCTCGAGTGCAGCGTATTTAGCCG (SEQ ID NO: 16). The upstream primer for feline soluble TRAIL: N8071H10-TACATGGTAAGAGAAAGAGGTCCTCAGAGAGTAGCA (SEQ ID NO: 17). The downstream primer which created fusion with V5 tag and His tag in the vector: R1886B10-CCTCGAGTGTAGCCGATTAAAAAGGCCCCGAAAAAAC (SEQ ID NO: 18). The downstream primer without creating V5-His tag, R4429A11-CCTCGAGTTTAGCCGATTAAAAAGGCCCCGAAAAAAC (SEQ ID NO: 19). For both canine and feline soluble TRAIL genes, 5′-RACE-Ready DNA and 3′-RACE-Ready DNA (see above) were used as templates for RCR reactions. In all the PCR reactions, the DNA polymerase used is Advantage HF (Invitrogen Inc., Carlsbad, Calif.) and the PCR conditions were as follows: 5 minutes at 95° C. to denature the template, followed by 30 cycles of 30 seconds at 94° C. and 1 minute at 55° C. and 1.5 minutes at 68° C., followed by 7 minutes at 74° C. The PCR products were analyzed by electrophoresis. The above PCR product was then cloned into pUniN5-His-TOPO cloning vector (Invitrogen, Carlsbad, Calif.) following manufacturer's instructions. About six independent clones containing inserts of the expected size (0.6 kb) were sequenced (Advanced Genetic Analysis Center, St. Paul, Minn.) and they all have complete correct sequence when compared with canine RACE sequence.

The soluble version of human TRAIL protein, which contains the extracellular portion of the gene, was demonstrated to possess apoptosis-inducing activity for cancer cells, i.e. tumor killing activity. This form of protein can be conveniently produced in bacteria. To clone the soluble version of canine and feline TRAIL genes, primers were designed based on the sequence homology with human protein sequence and first-strand cDNA was used as templates. The extracellular portion of each gene (human, canine and feline TRAIL) was cloned into pUniNV5-His-TOPO cloning vector (Invitrogen, Carlsbad, Calif.). Two versions of clones were designed : one with protein fusion at its C-terminal with V5-His tag of the vector; the other contained only the native protein without tags. Six independent clones of each construct (six total) with the correct size of insert were sent and confirmed by sequence analysis. They are designated as follows: pUni/V5-His-sh-huTRAIL, pUni-sh-huTRAIL, pUniN5-His-sh-caTRAIL, pUni-sh-caTRAIL, pUniNV5-His-sh-feTRAIL, pUni-sh-feTRAIL.

### EXAMPLE 2

### Cloning of Soluble Trail Genes Into Expression Vectors

In the Example presented in this section, studies on subcloning canine and feline TRAIL into various expression vectors are described.

1. Subcloning of HA-tagged canine and feline TRAIL into mammalian expression vector. The soluble portion canine and feline TRAIL gene was subcloned into pDisplay vector by PCR amplification of soluble canine and feline TRAIL genes in pUniNV5-His-TOPO cloning vector: 5′ primer for canine TRAIL gene, S2633B10-GCCAGATCTGTAAGCGACCGAGGTTCTCAG (SEQ ID NO: 32); 5′ primer for feline TRAIL gene, S2633B09-GCCAGATCTGTAAGAGAAAGAGGTCCTCAG (SEQ ID NO: 33), 3′ primer, S2633B11-AAAACTGCAGTTAGCCGATTAAAAAGGCCCCG (SEQ ID NO: 34). To facilitate cloning, two restriction enzyme sites Bgl II (5′ primer) and Pst I (3′ primer) were incorporated into the primer sequences as shown by underline. The insert was fused in-frame to the signal peptide and HA epitope sequences present in the vector. The stop codon TAA (shown in bold) of TRAIL was included in the 3′ primer to terminate translation, therefore the vector-encoded PDGFR transmembrane domain downstream of the insert was not translated in the final plasmid construct (pDisplay-HA-ca-TRAIL and pDisplay-HA-fe-TRAIL).

2. Subcloning of canine TRAIL (without HA tag). Canine and feline TRAIL was subcloned into pSecTag2 B vector by PCR amplification of soluble canine and feline TRAIL genes in pUniNV5-His-TOPO cloning vector using primers: 5′ primer for canine TRAIL gene, S2633B06-GCTTGGTACCGTAAGCGACCGAGGTTCTCAG (SEQ ID NO: 35); 5′ primer for feline TRAIL gene, S2633B08-GCTTGGTACCGTAAGAGAAAGAGGTCCTCAG (SEQ ID NO: 36), 3′ primer, S2633B07-CTCCTCGAGTTAGCCGATTAAAAAGGCCCC (SEQ ID NO: 37). To facilitate cloning, two restriction enzyme sites Kpn 1 (5′ primer) and Xho I (3′ primer) were incorporated into the primer sequences as shown by underline. The insert was fused in-frame to the signal peptide sequences present in the vector. The stop codon TAA (shown in bold) of TRAIL was included in the 3′ primer to terminate translation. The final plasmid construct was designated pSecTag2-ca-TRAIL and pSecTag2-fe-TRAIL.

3. Cloning of soluble human, canine and feline TRAIL into bacterial expression vector, pBAD-Thio-E. Human, canine and feline TRAIL genes in pUni-V5-His-TOPO cloning vectors: pUniNV5-His-sh-huTRAIL, pUni-sh-huTRAIL, pUniNV5-His-sh-caTRAIL, pUni-sh-caTRAIL, pUni/V5-His-sh-feTRAIL, pUni-sh-feTRAIL were recombined with pBAD/Thio-E, in the presence of Cre Recombinase, according to Invitrogen's manufacture instruction. The new bacterial expression plasmids put TRAIL genes under bacterial promoter pBAD, which was inducible by arabinose. There was a Thio-protein (Thioredoxin) fusion at the N-terminals of all the proteins. The recombination reaction was carried out at 37° C. for 30 minutes and terminated at 65° C. for 5 minutes. Three independent clones of each construct with correct restriction analysis pattern were sent for sequence confirmation. Those plasmids are designated as follows: pBAD-Thio-V5His-sh-huTRAIL, pBAD-Thio-sh-huTRAIL, pBAD-Thio-V5His-sh-caTRAIL, pBAD-Thio-sh-caTRAIL, pBAD-Thio-V5His-sh-feTRAIL, pBAD-Thio-sh-feTRAIL.

4. Cloning of soluble human, canine and feline TRAIL into bacterial expression vector, pCRT7-E. Human, canine and feline TRAIL genes in pUni-V5-His-TOPO cloning vectors: pUniNV5-His-sh-huTRAIL, pUni-sh-huTRAIL, pUniNV5-His-sh-caTRAIL, pUni-sh-caTRAIL, pUniNV5-His-sh-feTRAIL, pUni-sh-feTRAIL feTRAIL were recombine with pCRT7-E, in the presence of Cre Recombinase, according to Invitrogen's manufacture instruction. The new bacterial expression plasmids put TRAIL genes under bacterial promoter pT7, which is inducible by IPTG (Isopropyl bata-D-Thiogalactopyranoside). The recombination reaction was carried out at 37° C. for 30 minutes and terminated at 65° C. for 5 minutes. Three independent clones of each construct with correct restriction analysis pattern were sent for sequence confirmation. Those plasmids are designated as follows: pCRT7-V5His-sh-huTRAIL, pCRT7-sh-huTRAIL, pCRT7-V5His-sh-caTRAIL, pCRT7-sh-caTRAIL, pCRT7-V5His-sh-feTRAIL, pCRT7-sh-feTRAIL.

5. Cloning of soluble canine and feline TRAIL into bacterial expression vector, pBAD/His A. Canine and feline TRAIL genes in pUni-V5-His-TOPO cloning vectors (pUni[V5-His-sh-caTRAIL, pUni-sh-caTRAIL, pUniNV5-His-sh-feTRAIL and pUni-sh-feTRAIL) were used as DNA template in PCR reactions. The new bacterial expression plasmids put TRAIL genes under bacterial promoter pBAD, which is inducible by arabinose. Only the native TRAIL proteins will be expressed and there is no Thio protein fusion at N-terminal. The PCR reaction was carried out with Pfu as polymerase and as follows: 5 minutes at 94° C. to denature the template, followed by 30 cycles of 45 seconds at 94° C. and 45 seconds at 52° C. and 1.5 minutes at 72° C., followed by 10 minutes at 72° C. The primers used: canine TRAIL upstream: 43114-001-GAATTGCCCTTATTCCTTCCATGGTAAGCGACCGAGGTTCT (SEQ ID NO: 38). The canine TRAIL downstream primer without generating V5-His tag fusion: 43114-003-TGTTTTTTCTCGAGTGCACTGCAGTTAGCCGATTAAAAAGG (SEQ ID NO: 39). The canine TRAIL downstream primer which generates V5-His tag fusion: 43114-004-CACAGTCGAGGCTGATAGCTGCAGTCAATGGTGATGGTGATG (SEQ ID NO: 40). Feline TRAIL upstream: 43114-002-CTCGAGGAATTGCCCTTTCCATGGTAAGAGAAAGAGGTCCT (SEQ ID NO: 41). The feline TRAIL downstream primer without generating V5-His tag fusion: 43114-005-CTCCCGAATTGCCCTTCCCTGCAGTTAGCCGATTAAAAAGG (SEQ ID NO: 42). The feline TRAIL downstream primer which generates V5-His tag fusion: 43114-006-CACAGTCGAGGCTGATAGCTGCAGTCAATGGTGATGGTGATGATG (SEQ ID NO: 43). After visualizing the PCR bands on the electrophoresis analysis, the PCR reactions were purified with Qiagen PCR Purification Kit. The purified DNA was then digested with Nco I and Pst I at 37° C. for three hours. Digested PCR DNA fragments were then excised and purified from electrophoresis gel using Qiagen's gel extraction kit. The gel purified PCR fragments were then ligated with Nco I and Pst I double digested plasmid vector, pBAD/Myc-His in the presence of T4 DNA ligase. The ligation reaction was carried out at room temperature for one hour. Three independent clones of each construct with correct restriction analysis pattern were sent for sequence confirmation. Those plasmids are designated as follows: pBAD-V5His-sh-caTRAIL, pBAD-sh-caTRAIL, pBAD-V5His-sh-feTRAIL and pBAD-sh-feTRAIL.

### EXAMPLE 3

### Expression of Trail Genes In Mammalian Cells

In the Example presented in this section, studies are described that identify methods to express and assay the novel canine and feline TRAIL genes expressed in mammalian cells.

1. Transfection of TRAIL. Human 293 cells grown in 6 well plate were transfected with 2.5 ug of plasmids encoding canine or feline TRAIL using Lipofectamine Plus (Gibco-BRL, Gaithersburg, Md., Cat. No. 10964-013).

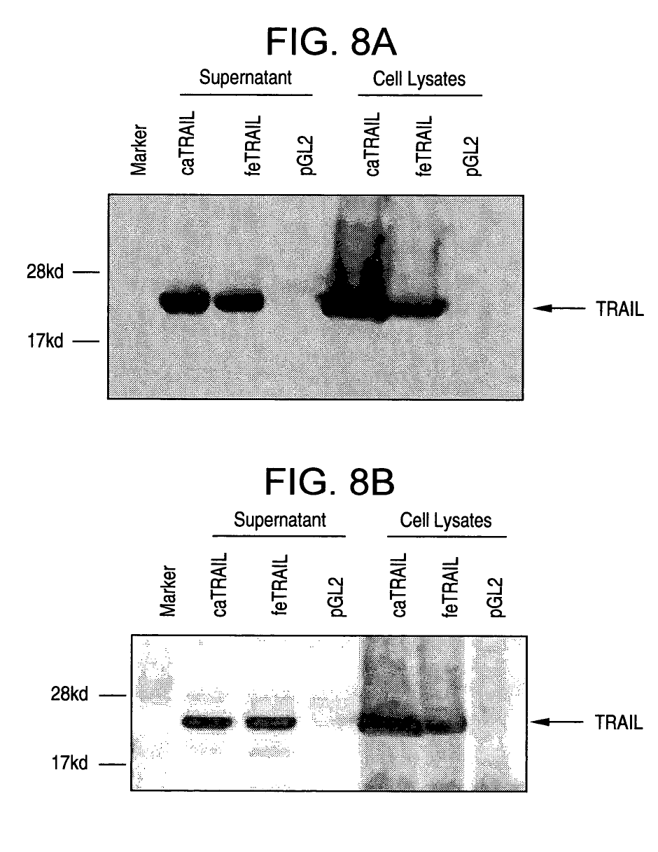

2. Detection of TRAIL by immunoblot analysis. 2 days post-transfection with plasmids pDisplay-HA-ca-TRAIL and pDisplay-HA-fe-TRAIL, cells were harvested by lysis in NuPAGE LDS Sample Buffer (NOVEX, San Diego, Calif.). The culture supernatants were harvested by centrifugation at 3000 rpm for 15 minutes. The proteins were separated by 10% Bis-Tris gel with MES running buffer and transferred to nitrocellulose membrane (NOVEX, San Diego, Calif.). For immunoblot analysis, HA antibody against HA epitope or anti-human TRAIL antibody (R & D Systems, cat. no. AF375) were diluted 1:1000 and incubated with the blot for 1.5 hours. After incubating for 30 minutes with alkaline phosphatase conjugated anti-mouse IgG (1:1000, Boehringer Mannheim, Indianapolis, Ind.), the bound antibody was detected using phosphatase substrate BCIP/NBT (KPL, Gaithersburg, Md.). The expression of transfected TRAIL was clearly detected from both cell lysates and culture supernatants (FIG. 8)

The cDNAs encoding canine and feline TRAIL were also subcloned into mammalian expression vector pSecTag2, creating pSecTag2-ca-TRAIL and pSecTag2-fe-TRAIL. Human 293 cells were transfected with these plasmids. The cells were harvested 48 hours post transfection for analysis. The expression of TRAIL was studied by Western immunoblot analysis. TRAIL expression was detected from cell lysates but not culture supernatants (data not shown).

Various anti-human TRAIL antibodies were tested for their cross-reactivity with canine or feline TRAIL proteins. Table 1 shows the results of the tests. AF375, anti-human TRAIL goat antisera (R & D Systems) demonstrated the strongest reactivity with both canine and feline TRAIL proteins. The antibodies from Sigma and Upstate Biotechnology did not have visible cross-reactivity for both canine and feline TRAIL.

3. MTT Assay for cell growth inhibition. Cell proliferation assay Kit (Roche, Cat. # 1 456 007) was used for this assay. U937 or any other types of cells were centrifuged at 2,000 rpm for 10 minutes. The cells were then resuspended in the complete growth medium and adjust cell counts at 2XI0⁵ cells/ml. 50 ul of cells (1×10⁴) and 50 ul of supernatant or other solutions containing apoptosis inducing agents (e.g. TRAIL) were used in 96-well plate. After 24 or 48 hrs of incubation at 37° C., 10 ul MTT solution was added to each well and the plate was incubated in CO₂ incubator for 4 hours. 100 ul MTT solvent was then added. Plates were left in 37° C. incubator overnight to allow MTT crystals to completely dissolve. Then the plates were read at 570 nm and 690 nm (background) on ELISA plate reader.

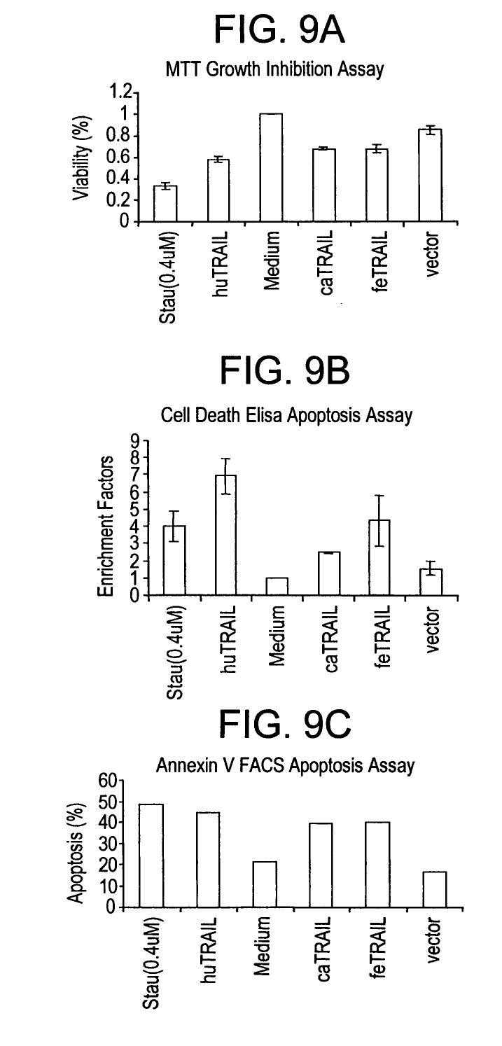

One important feature of TRAIL is its ability to specifically induce apoptosis and inhibit cell growth of tumor cells. U937 human tumor cells were grown in the presence or absence of TRAIL proteins produced from conditioned media of transfected 293 cells. The proliferation rate was normalized against control cells which were treated normal growth medium. Another control experiment was carried out with conditioned media from green fluorescent protein (GFP) transfected cells. The results from two independent experiments were summarized in FIG. 9A. The addition of HA-tagged canine and feline TRAIL inhibited the cell growth of U937 cells over 35% of that of control. The inhibitory activity seen with HA-tagged canine and feline TRAIL was comparable to that with the human commercial TRAIL proteins (Upstate Technologies, 40% level of controls).

4. Cell Death Detection ELISA Plus assay for apoptosis. The assay is based on a quantitative sandwich-enzyme-immunoassay-principle using mouse monoclonal antibodies directed against DNA and histones, respectively. This allows the specific determination of mono- and oligonucleosomes in the cytoplasmatic fraction of cell lysates. Roche kit, Cat. # 1 774 425, was used for this assay. 0.1 ml of U937 or any other types of cells (2×104 ) and 0.1 ml of tissue culture supernatant or other solutions containing apoptosis inducing agents (e.g. TRAIL) were used for this assay in 48-well plate. After 24 or 48 hrs of incubation at 3 7° C., the cells were spun at 2,000 rpm for 10 minutes. Cell pellet was then resuspended in 200 ul lysis buffer and incubated for 30 minutes at room temperature. The cell lysate was then centrifuged at 2,000 rpm for 10 minutes and diluted at 1:10 with incubation buffer. 20 ul of diluted lysate was added to coated plate (MTP from kit) and 80 ul of the immunoreagent was added to each well. MTP was then covered with adhesive cover foil and incubated under gentle shaking for 2 hours at room temperature. The solution was then removed thoroughly by tapping and subsequently rinsed 3 times with 250 ul incubation buffer. 100 ul ABTS solution was added and incubated on a plate shaker at 250 rpm for 15 minutes. Readings at 405 nm and 490 nm (background) were done on ELISA reader.

U937 human tumor cells were grown in the presence or absence of TRAIL proteins produced from conditioned media of transfected 293 cells. The apoptosis-induced cell death was measure in this assay. One control experiment was conducted with normal growth medium. Another control experiment was carried out with conditioned media from green fluorescent protein (GFP) transfected cells. The results from two independent experiments were summarized in FIG. 9B. The addition of HA-tagged canine and feline TRAIL induced apoptosis specific cell death of U937 cells 2.5 and 4 times that of control, respectively. The inhibitory activity seen with HA-tagged canine and feline TRAIL was comparable to that with the human commercial TRAIL proteins (Upstate Technologies, 7 times of controls).

5. Annexin V apoptosis assay. ApoScreen Annexin V Apoptosis Kit (Cat. No. 10010-02, Southern Biotechnology Assoc. Inc.) was used for this assay. Annexin V detects the change in position of phosphatidy serine (PS) in the cell membrane. In non-apoptotic cells, most phosphatidyl serine molecules are at the inner layer of the plasma membrane. After inducing apoptosis, PS redistributes to the outer layer of the membrane. PS translocation precedes other apoptotic events allowing early detection of apoptosis. 1 ml of U937 cells (2×10⁴) or any other types of cells and 1 ml of supernatant or other solutions containing apoptosis inducing agents (e.g. TRAIL) were used for assay in 6-well plate. After 48 hrs of incubation, cells were washed in 2 ml of cold PBS (Phosphate-buffered saline) and resuspended in 100 ul of cold 1× binding buffer. 10 ul of Annexin V-FITC was added to each tube and the tube was gently vortexed and subsequently incubated for 15 minutes in the dark at 4° C. 200 ul of cold 1× binding buffer was added to each tube and 10 ul-of PI was also added to each tube. The staining was then analyzed by flow cytometry.

U937 human tumor cells were grown in the presence or absence of TRAIL proteins produced from conditioned media of transfected 293 cells. The apoptosis of U937 cells induced by conditioned media was measured in this assay. One control experiment was done with normal growth medium. Another control experiment was carried out with conditioned media from green fluorescent protein (GFP) transfected cells. The results from two independent experiments were summarized in FIG. 9C. While medium and conditioned media from GFP transfected cells induce background apoptosis 15-20%, the addition of HA-tagged canine and feline TRAIL induced apoptosis of U937 cells at 40%. The apoptosis inducing activity seen with HA-tagged canine and feline TRAIL was comparable to that with the human commercial TRAIL proteins (Upstate Technologies, 45% of apoptosis).

### EXAMPLE 4

### Expression of Trail Genes Under pBAD-Thio Promotor in Bacterial Cells

In the Example presented in this section, studies are described that identify methods to express and assay the novel canine TRAIL as well as human TRAIL genes expressed in bacterial cells.

1. Expression of soluble TRAIL proteins under pBAD-Thio promoter in bacteria. Bacterial expression plasmids, pBAD-Thio-V5His-sh-huTRAIL, pBAD-Thio-sh-huTRAIL, pBAD-Thio-V5His-sh-caTRAIL, pBAD-Thio-sh-caTRAIL, pBAD-Thio-V5His-sh-feTRAIL, pBAD-Thio-sh-feTRAIL, were transformed into E. coli TOP 10 cells (Invitrogen, Calif.). Single colonies were inoculated into 4 ml of L broth containing 50 ug/ml of Kanamicin (L50Kan broth) grown in 37° C. shaker overnight. 0.1 ml of these overnight cultures was inoculated into 4 ml fresh L50kan broth and shaked at 37° C. for 2 hours. At this point, three different concentrations of arabinose were added to each culture: 0.2%, 0.02% and 0.002%. After the cultures were shaked for 4 more hours at 37° C., 1 ml of each culture was centrifuged and the bacterial pellets were lysed in 100 ul of 1×sample buffer at 70° C. for 10 minutes. 15 ul of those lysate samples were loaded onto 10% Bis-Tris gel (Invitrogen, NuPAGE 1.0 mm×15 well, NP0303) and stained with 0.25% (w/v) Brilliant Blue R in 40% (v/v) methanol and 7% (v/v) acetic acid.

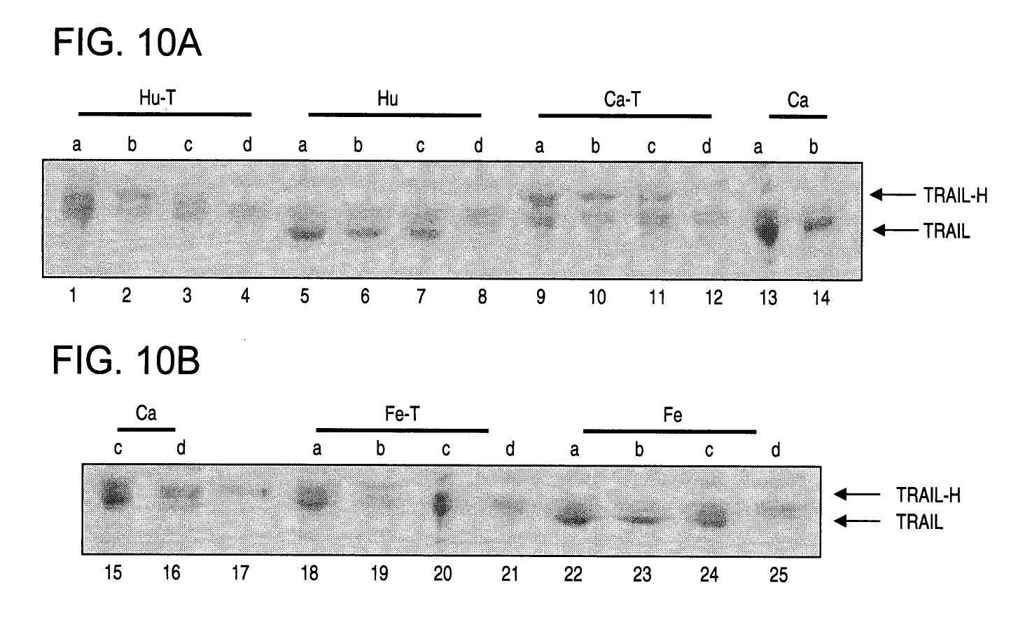

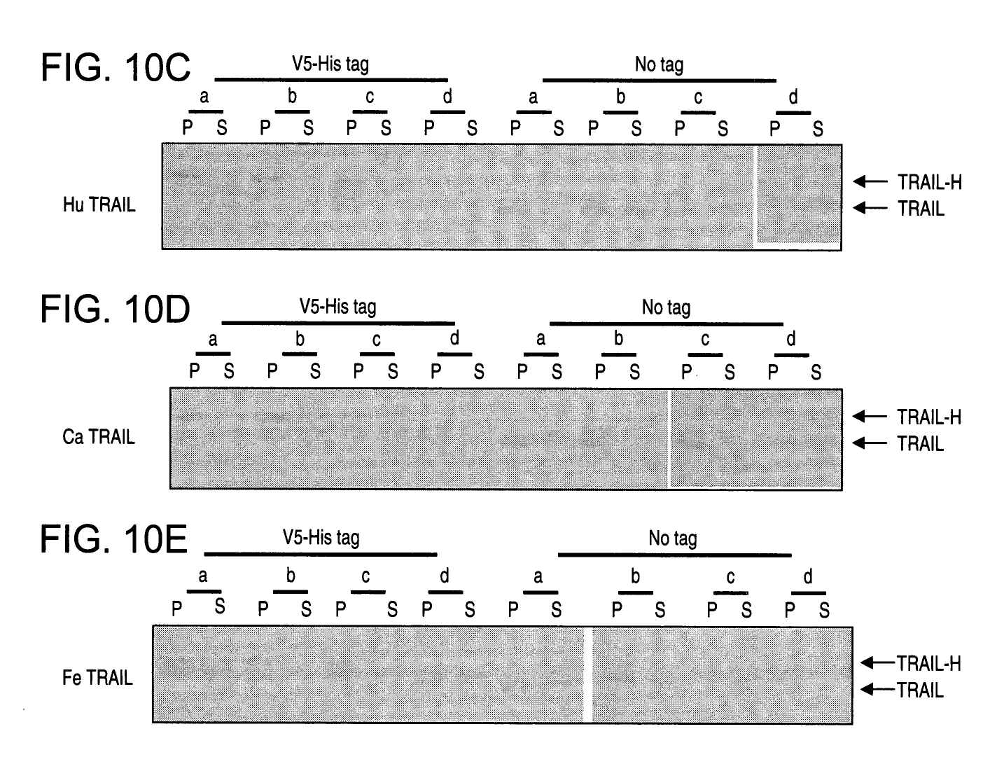

There was very clear induction of TRAIL-Thio protein expression for all six constructs (human with and without tag; canine with and without tag and feline with and without tag) with all the concentrations of arabinose (FIGS. 10A-10B). The identity of these TRAIL-Thio proteins was further confirmed by Western immunoblot analysis using anti human TRAIL antibody (cat. no. AF375, R & D Systems, data not shown) as well as anti-V5 antibodies (cat. no. 46-0708, Invitrogen, data not shown).

2. Solubility of soluble TRAIL-Thio proteins under pBAD-Thio promoter in bacteria. The solubility of TRAIL proteins expressed was examined by twice of freezing in dry ice and thawing at 37° C. waterbath of the bacterial lysate, followed by sonication (Branson Sonifier 250) at 20% duty cycle with output control of 3. The whole lysate was then centrifuged for 1 minutes at 12,000 rpm to separate soluble fraction (S) from insolubles, i.e. pellet (P). Both fractions were mixed with protein sample buffer. Equivalent volumes of both fractions were loaded onto 10% Bis-Tris gel (Invitrogen, Calif.) and stained with 0.25% Brilliant Blue R. Even though more than 50% of TRAIL-Thio proteins present in the insoluble fractions, there was significant amount of TRAIL-Thio in the soluble fractions (FIGS. 10C-10E).

3. Purification of TRAIL-Thio proteins expressed under pBAD-Thio promoter. 300 ml of bacteria culture expressing human or canine TRAIL proteins under pBAD-Thio promoter were centrifuged and the cell pellets were lysed with Anderson Emulsi Flex-C5 mechanical lysis devise into 30 ml of lysis buffer. The lysates were centrifuged at 10,000 rpm for 20 minutes and then again at 18,000 rpm for 45 minutes. 25 ml of the supernatant was loaded onto the prewet His-band Quick 900 Cartridges, following manufacture's instructions (Novagen). The initial flow-through was re-loaded to the columns to further enhance the binding of TRAIL protein to the columns. Two washes were followed: first with 20 ml of 1× binding buffer (5 mM imidazole, 0.5 M NaCL, 20 mM Tris-HCL, pH 7.9 and 0.1% Triton X-100), then with 15 ml of 0.5× wash buffer (30 mM imidazole, 0.25 M NaCL, 10 mM Tris-HCL, pH 7.9 and 0.1% Triton X-100). 4 ml of Elute buffer (1 M imidazole, 0.5 M NaCL and 20 mM Tris-HCL pH 7.9) was added to the column and 8 fractions (0.5 ml/tube) were collected.

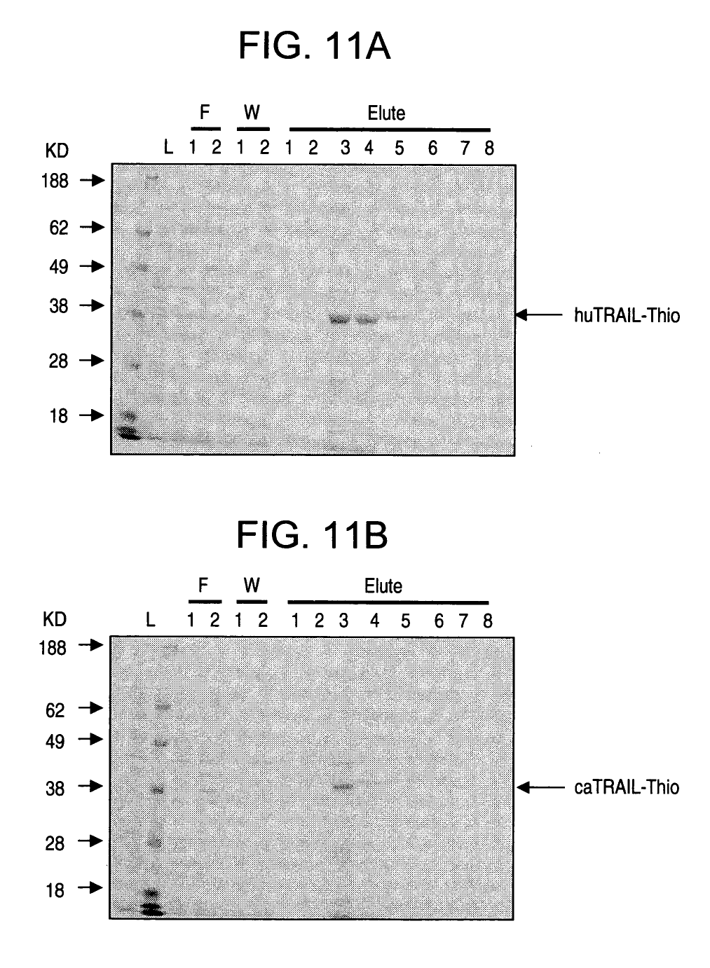

6 ul of each fraction was loaded onto 10% Bis-Tris gel and stained with 0.25% Brilliant Blue R. Both human and canine TRAIL-Thio proteins were clearly visible in fractions 3 and 4 (FIGS. 11A-11B). 4 samples of fractions (hu-E3, hu-E4, ca-E3 and caE2+E4) were enlarged to the volume of 10 ml and dialyzed against 2 liters of 1× PBS at 4° C. overnight. The dialysis apparatus used is Slide-A-Lyzer cassette (7,000 MWCO, 3-12 ml, PIERCE, Rockford, Ill.). The final dialyzed protein samples were added to the final concentration of 0.1 mM DTT and stored at −20° C.

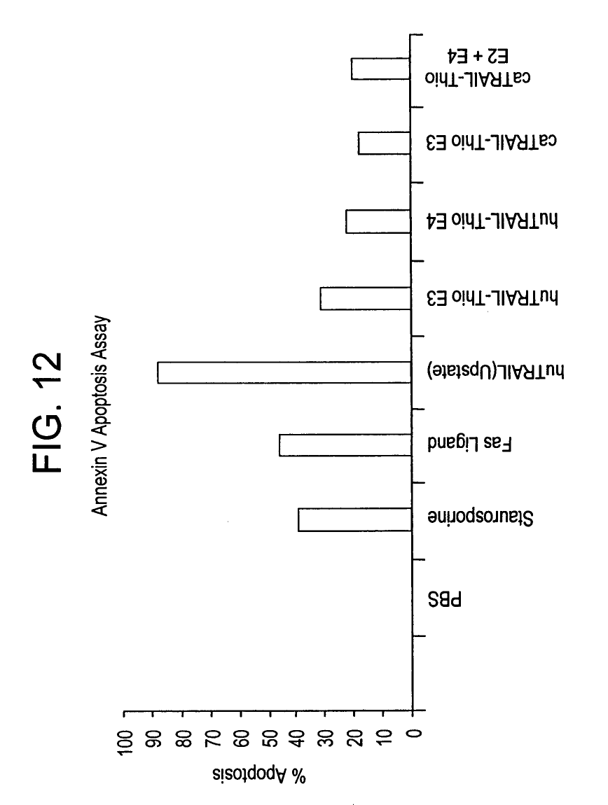

4. Induction of apoptosis of human tumor cells by bacteria expressed TRAIL-Thio in Annexin V assay. U937 human tumor cells were grown in the presence or absence of TRAIL-thio proteins semi-purified as mentioned above. The apoptosis of human tumor cells U937 induced by those proteins was measured in this assay. While negative control PBS barely induced any background apoptosis, both canine and human TRAIL-thio induced apoptosis of U937 cells about 20-30% (FIG. 12). The apoptosis inducing activity of human commercial TRAIL proteins (Upstate Technologies) was very strong in this experiment (above 85%).

### EXAMPLE 5

### Expression of Trail Genes Under T7 Promotor in Bacterial Cells

In the Example presented in this section, studies are described that identify methods to express and assay the novel feline and canine TRAIL genes expressed in bacterial cells.

1. Expression of soluble TRAIL proteins under T7 promoter in bacteria. Bacterial expression plasmids, pCRT7-V5His-sh-huTRAIL, pCRT7-sh-huTRAIL, pCRT7-V5His-sh-caTRAIL, pCRT7-sh-caTRAIL, pCRT7-V5His-sh-feTRAIL, pCRT7-sh-feTRAIL were transformed into E. coli BL21 cells (Invitrogen, Calif.). Single colonies were inoculated into 4 ml of L broth containing 50 ug/ml of Kanamicin and 34 ug/ml of Chloramphenicol (L50Kan34Cm broth) grown in 37° C. shaker overnight. 3.2 ml of these overnight cultures were inoculated into 150 ml fresh L50kan34Cm broth and shaked at 30° C. for 3 hours. At this point, IPTG solution was added to each culture to the final concentration of 1 mM. After the cultures were shaked for 2.5 more hours at 30° C., 1 ml of each culture was centrifuged and the bacterial pellets were lysed in 100 ul of 1× sample buffer at 70° C. for 10 minutes. 15 ul of those lysate samples were loaded onto 10% Bis-Tris gel (Invitrogen, NuPAGE 1.0 mm×15 well, NP0303) and stained with 0.25% (w/v) Brilliant Blue R in 40% (v/v) methanol and 7% (v/v) acetic acid.

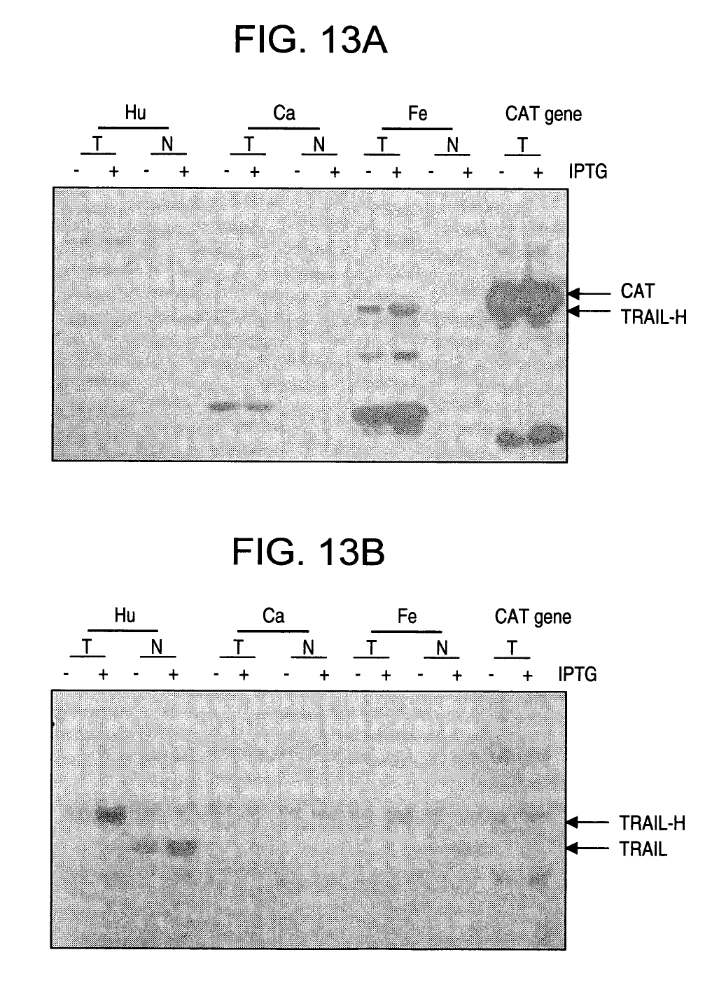

When analyzed by Brilliant Blue R stained gel, there is no clear induction of TRAIL protein expression for all six constructs (human with and without tag; canine with and without tag and feline with and without tag) with the presence of IPTG (data no shown). The same samples were further analyzed by Western immunoblot analysis using anti-V5 (Invitrogen) and anti-human TRAIL antibody (AF375, R & D Systems) as probe. There is a slight induction of feline and human TRAIL protein expression with the presence of IPTG (FIGS. 13A and 13B). Even for the positive control, CAT expression was only slightly enhanced with IPTG. There is no canine TRAIL protein expressed, V5-His tagged or non-tagged, despite several attempts on several independent clones. There was no sequence error found with those two canine TRAIL constructs.

2. Solubility of soluble TRAIL proteins under pT7 promoter in bacteria. 3 liters of bacterial cultures were centrifuged and the cell pellets were lysed with Anderson Emulsi Flex-C5 mechanical lysis devise into 300 ml of lysis buffer. The lysates were centrifuged at 9,000 rpm for 30 minutes (1^{st} pellet) and then again at 19,000 rpm for 90 minutes (2^{nd} pellet). The supernatant was then loaded onto an anion exchange Q Fast-Flow column which was equilibrated with 0.1 M Tris, pH 8.0. The flow-through was then dialyzed in 50 mM NaPO₄, pH 6.8 in a dialysis bag with 3K MWCO, followed by centrifugation for 90 minutes at 19,000 rpm (3^{rd} pellet) and passing a 0.22 um filter. 50 ml of above sup was then loaded onto a cation exchange S-sepharose column (Hi Trap™ SP HP, Amershan Pharmacia) which was equilibrated with 50 mM NaPO₄, pH 6.5. The column was washed with 5 ml of 50 mM NaPO₄, pH 6.5 and eluded with 8 ml of 50 mM PO₄, pH 6.5 plus 1 M NaCL. All samples were mixed with SDS protein sample buffer and loaded onto 10% Bis-Tris gel (Invitrogen, Calif.), which was then stained with 0.25% Brilliant Blue R or proceed further for Western analysis.

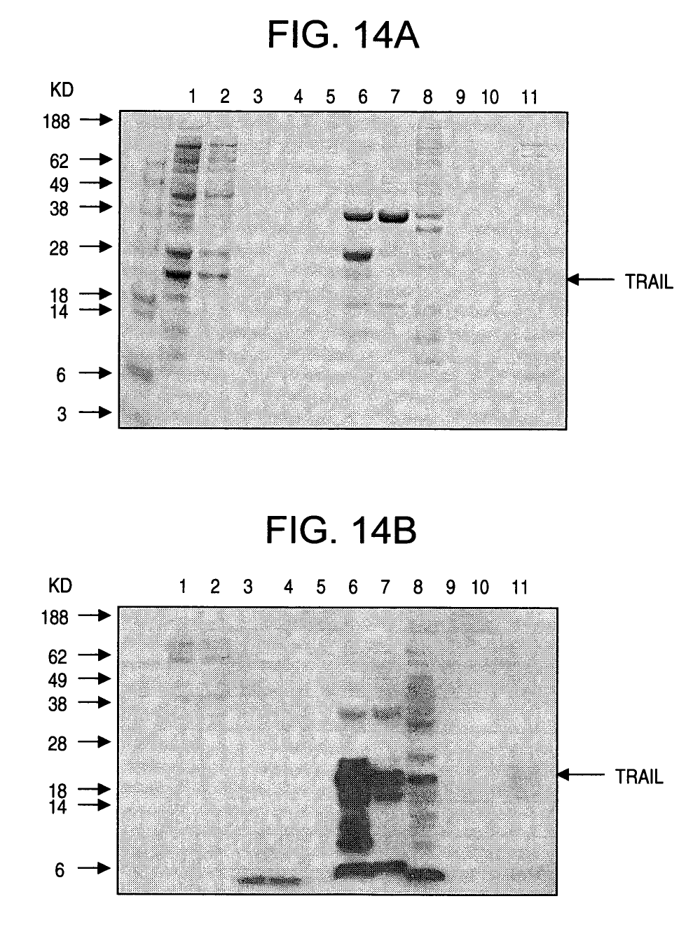

The solubility of TRAIL expressed from T7 promoter seems to be very poor and the majority of the TRAIL proteins were present in lysate pellets, i.e. insoluble fractions (FIGS. 14A and 14B).

### EXAMPLE 6

### Expression of Trail Genes Under pBAD Promotor in Bacterial Cells

In the Example presented in this section, studies are described that identify methods to express and assay the novel canine and feline TRAIL genes expressed in bacterial cells.

1. Expression of soluble TRAIL proteins under pBAD promoter in bacteria. Bacterial expression plasmids, pBAD-VSHis-sh-caTRAIL, pBAD-sh-caTRAIL, pBAD-V5His-sh-feTRAIL and pBAD-sh-feTRAIL, were transformed into E. coli TOP 10 cells (Invitrogen, Calif.). Single colonies were inoculated into 4 ml of L broth containing 100 ug/ml of Ampicillin (L100Amp broth) grown in 37° C. shaker overnight. 0.15 ml of these overnight cultures were inoculated into 4 ml fresh L100Amp broth and shaked at 37° C. for 2 hours. At this point, arabinose was added to the final concentration of 0.02%. After the cultures were shaked for 4 more hours at 37° C., 0.5 ml of each culture was centrifuged and the bacterial pellets were lysed in 150 ul of 1× sample buffer at 70° C. for 10 minutes. 12 ul of those lysate samples were loaded onto 10% Bis-Tris gel (Invitrogen, NuPAGE 1.0 mm×15 well, NP0303) and stained with 0.25% (w/v) Brilliant Blue R in 40% (v/v) methanol and 7% (v/v) acetic acid.

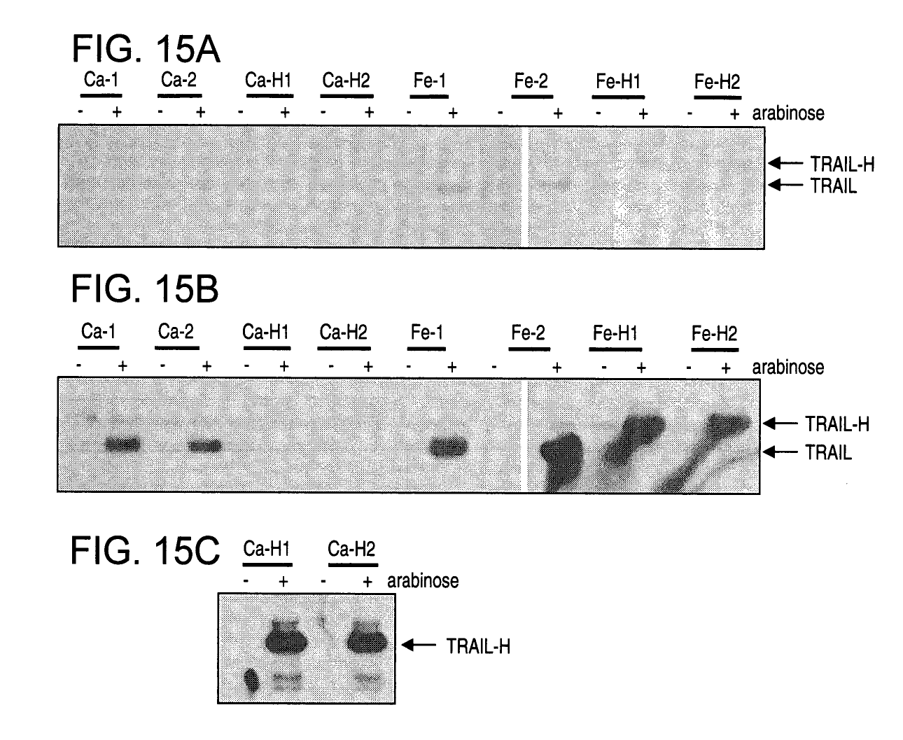

There was a very clear induction of TRAIL protein expression for feline constructs (with and without tag); the level for canine TRAIL protein expression was relatively low (FIG. 1 SA). The expression of canine TRAIL protein was clearly visible in Western immunoblot analysis using anti human TRAIL antibody (R & D Systems, FIG. 15B) as well as anti-V5 antibodies (Invitrogen, FIG. 15C).

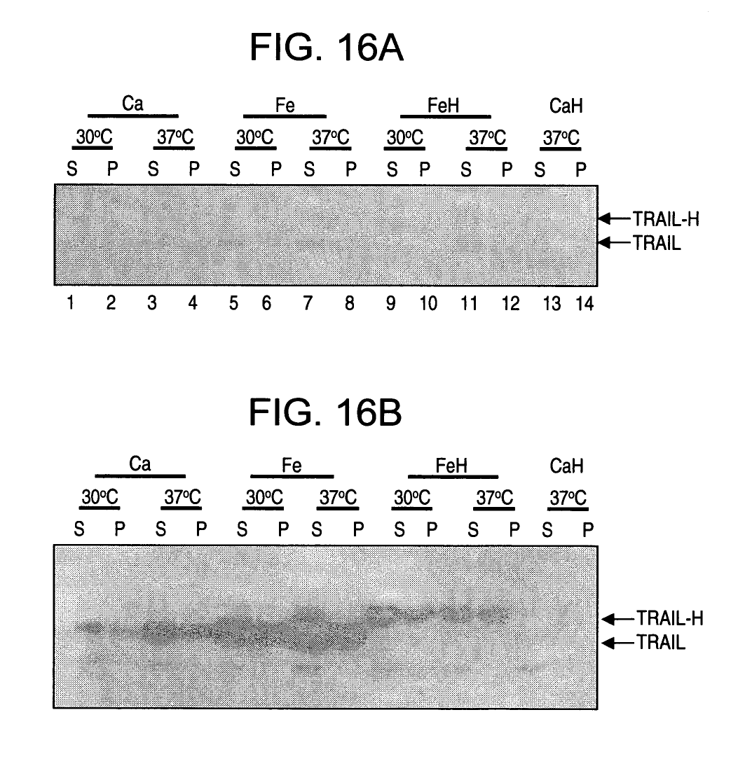

2. Solubility of soluble TRAIL proteins under pBAD promoter in bacteria. The solubility of TRAIL proteins expressed was examined by growing bacteria cultures at either 30° C. or 37° C. and twice of freezing in dry ice and thawing at 37° C. waterbath of the bacterial lysate, followed by sonication (Branson Sonifier 250) at 20% duty cycle with output control of 3 for 5 minutes. The whole lysate was then centrifuged for 5 minutes at 12,000 rpm to separate soluble fraction from insolubles. Both fractions were mixed with protein sample buffer. Equivalent volumes of both fractions were loaded onto 10% Bis-Tris gel (Invitrogen, Calif.) and stained with 0.25% Brilliant Blue R or analyzed by Western immunoblot with anti-TRAIl antibodies. The majority of TRAIL proteins were present in the soluble fractions at either 30° C. or 37° C. (FIGS. 16A and 16B). Furthermore, the level of soluble TRAIL proteins was more abundant at 37° C. than at 30° C.

3. Purification of TRAIL proteins expressed under pBAD promoter. 7 ml of bacteria culture expressing canine or feline TRAIL proteins under pBAD promoter were centrifuged and the cell pellets were resuspended into 4 ml of lysis buffer, followed by twice of freezing in dry ice and thawing at 37° C. waterbath and sonication (Branson Sonifier 250) at 20% duty cycle with output control of 3 for 5 minutes. The lysates were centrifuged at 12,000 rpm for 15 minutes. The supernatant was then loaded onto the prewet His-band Quick 900 Cartridges, following manufacture's instructions (Novagen). The initial flow-through was re-loaded to the columns to further enhance the binding of TRAIL protein to the columns. Two washes were followed: first with 20 ml of 1×binding buffer (5 mM imidazole, 0.5 M NaCL, 20 mM Tris-HCL, pH 7.9 and 0.1% Triton X-100), then with 15 ml of 0.5× wash buffer (30 mM imidazole, 0.25 M NaCL, 10 mM Tris-HCL, pH 7.9 and 0.1 % Triton X-100). 4 ml of Elute buffer (1 M imidazole, 0.5 M NaCL and 20 mM Tris-HCL pH 7.9) was added to the column and 8 fractions (0.5 ml/tube) were collected.

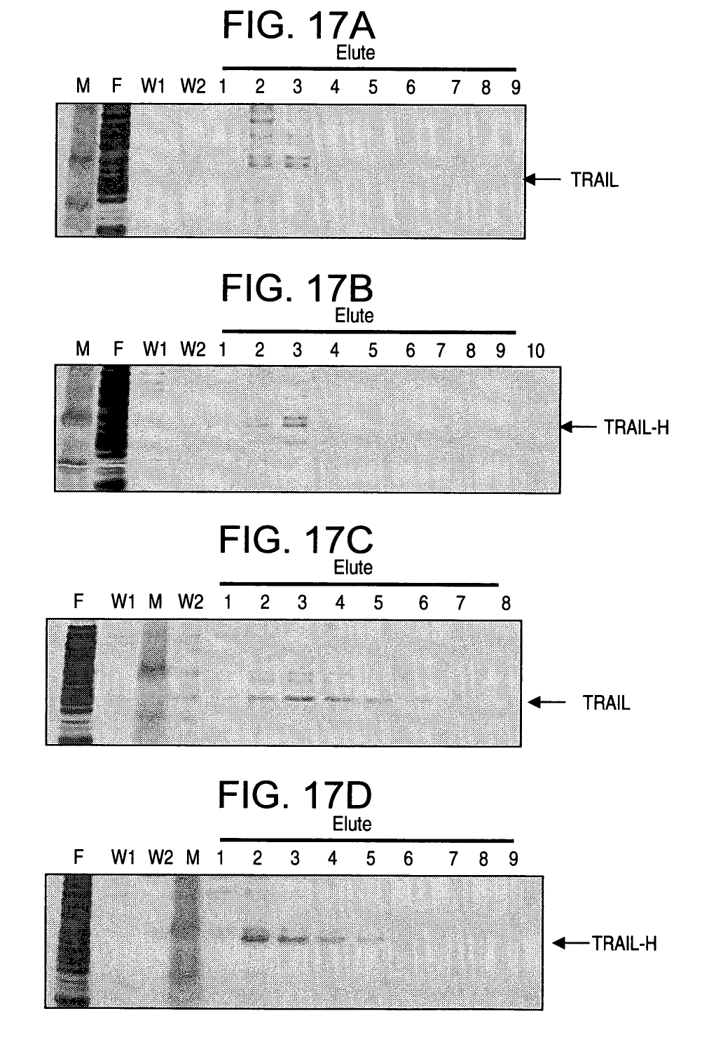

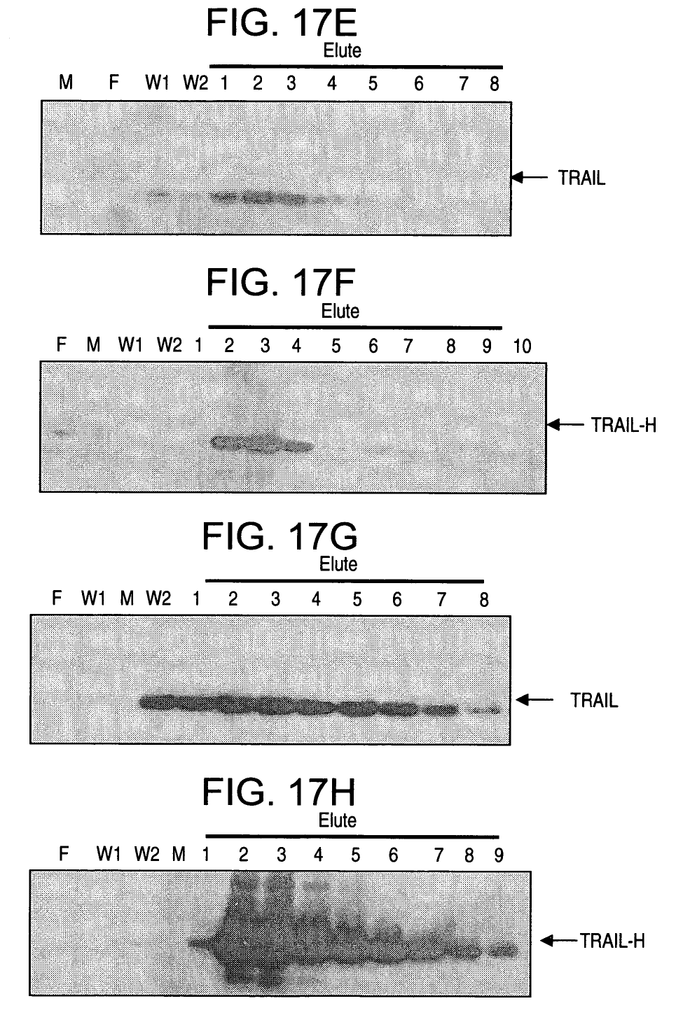

6 ul of each fraction was loaded onto 10 % Bis -Tris gel and stained with SilverXpress staining (Invitrogen, panel I, or analyzed by Western immunoblot with anti-TRAIL antibodies in panel II). For canine and feline TRAIL proteins with V5-His tag at their C-terminals, the eluted TRAIL was clearly visible in fractions 2, 3 and 4 (FIGS. 17B and. 17D). But for canine and feline TRAIL without any tag, the protein came off in a very broad peak, covering elution fractions 1 to 8 (FIGS. 17A and 17C). Furthermore, significant amount of TRAIL proteins was washed off with 0.5× wash buffer (lanes W2 of panels FIGS. 17A and 17C) for both canine and feline TRAIL proteins without any tags. Fractions were pooled together: canine TRAIL without V5-His tag (E2-6); canine TRAIL with V5-his tag (E2-7); feline TRAIL without V5-His tag (E1-8); feline TRAIL with V5-His tag (E2-8). Each sample was enlarged to the volume of 10 ml and dialyzed against 4 liters of 1× PBS at 4° C. overnight. The dialysis apparatus used is Slide-A-Lyzer cassette (7,000 MWCO, 3-12 ml, PIERCE, Rockford, Ill.). The final dialyzed protein samples were added to the final concentration of 0.1 mM DTT and stored at −20° C.

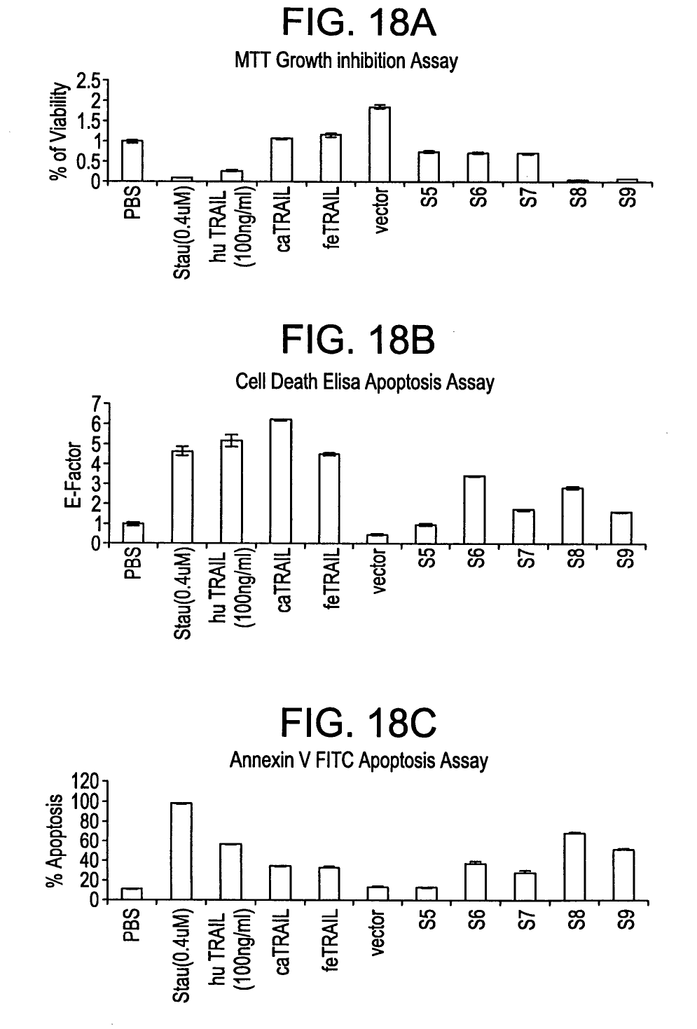

4. Inhibition of growth of human tumor cells by bacterial expressed TRAIL in MTT assay. U937 human tumor cells were grown in the presence or absence of TRAIL proteins expressed and purified from bacteria as described above. The proliferation rate was normalized against control cells which were treated normal growth medium. Another control experiment was carried out with PBS buffer. Both staurosporin (0.4 uM) and commercial human TRAIL (100 ng/ml) were assayed at the same time as positive controls. Also used in the assay are the mammalian expressed canine and feline TRAIL supernatants, as well as vector transfected supernatant. Five bacterial purified samples were assayed in this experiment. S5, bacterial column fractions which do not contain TRAIL, serve as negative control. S6, purified feline TRAIL from His-Band Quick 900 cartridges column. S7, purified feline TRAIL with V5-His tag. S8, semi-purified canine TRAIL, wash from the column (lanes W2 of FIG. 17A). S9, semi-purified feline TRAIL, wash from the column (lanes W2 of FIG. 17C). This result suggests that the addition of semi-purified canine and feline TRAIL inhibited the cell growth of U937 cells over 80% of that of control (FIG. 18A).

5. Induction of apoptosis of human tumor cells by bacterial expressed TRAIL in Cell Death ELISA assay. U937 human tumor cells were grown in the presence or absence of the same set of samples as used in the MTT growth inhibition assay. In addition to the S8 and S9, the semi-purified TRAIL protein samples, which was able to induce apoptosis of U937 cells, the more purified TRAIL samples, S6 and S7, were also positive in this assay (FIG. 18B).

6. Induction of apoptosis of human tumor cells by bacterial expressed TRAIL in Annexin V assay. U937 human tumor cells were grown in the presence or absence of the set of TRAIL protein samples as mentioned above. The apoptosis of human tumor cells U937 induced by those proteins was measured in this assay. Consistent with the results of Cell Death ELISA assay, S8 and S9, the semi-purified TRAIL protein samples, being able to induce apoptosis of U937 cells 7 times more than control sample S5 (FIG. 18C). Furthermore, the more purified TRAIL samples, S6 and S7, were also positive in this assay. The apoptosis inducing activity of human commercial TRAIL proteins (Upstate Technologies) was similar to the bacterial samples S8 and S9.

### EXAMPLE 7

### Apoptosis Inducing Activity of Trail for Various Cancer/Normal Cells

In the Example presented in this section, studies are described that identify the specificity of apoptosis inducing activity of TRAIL for various cancer/normal cells.

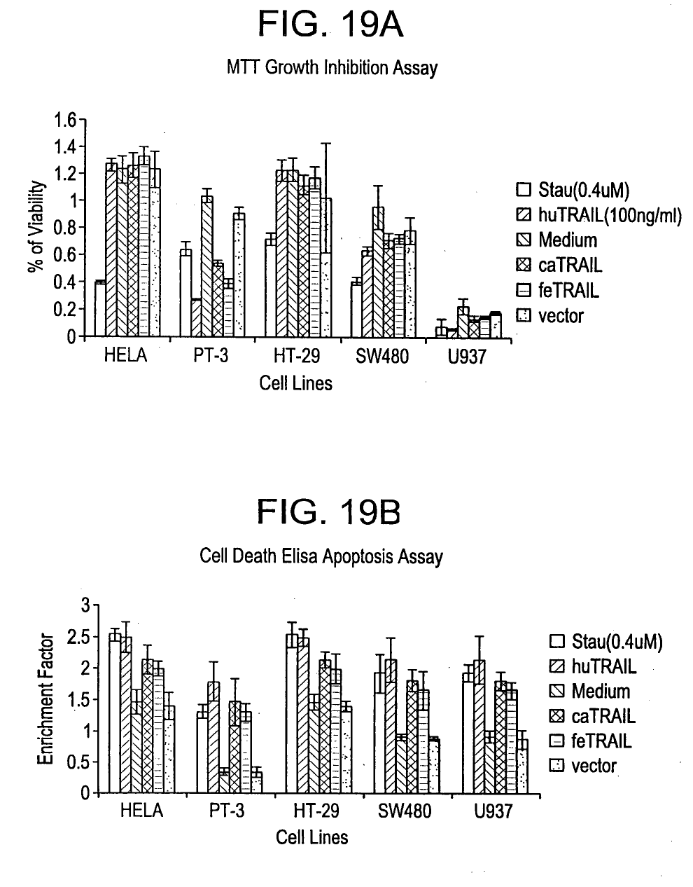

1. Apoptosis-inducing activity of canine and feline TRAIL for various human tumor cell lines. Five human tumor cell lines (HELA, cervix epitheloid carcinoma; PT-3, HT-29, colon adenocarcinoma; SW480, colon adenocarcinoma; and U937, histiocytic lymphoma) were assayed for their sensitivity against mammalian expressed canine and feline TRAIL proteins. Both MTT growth inhibition assay and cell death ELISA apoptosis assay were performed. For MTT assay, in addition to U937, another human tumor cell line, PT-3, was very sensitive to both canine and feline TRAIL induced apoptosis. The effect for SW480 was less clear (FIG. 19A).

Cell Death ELISA assay was also performed for the five human tumor cell lines. All five cell lines showed sensitivity to TRAIL induced apoptosis in this assay (FIG. 19B). Furthermore, both canine and feline TRAIL induced apoptosis very effectively. This result further confirmed that TRAIL is a potent anti-cancer therapeutic agent with broad therapeutic spectrum.

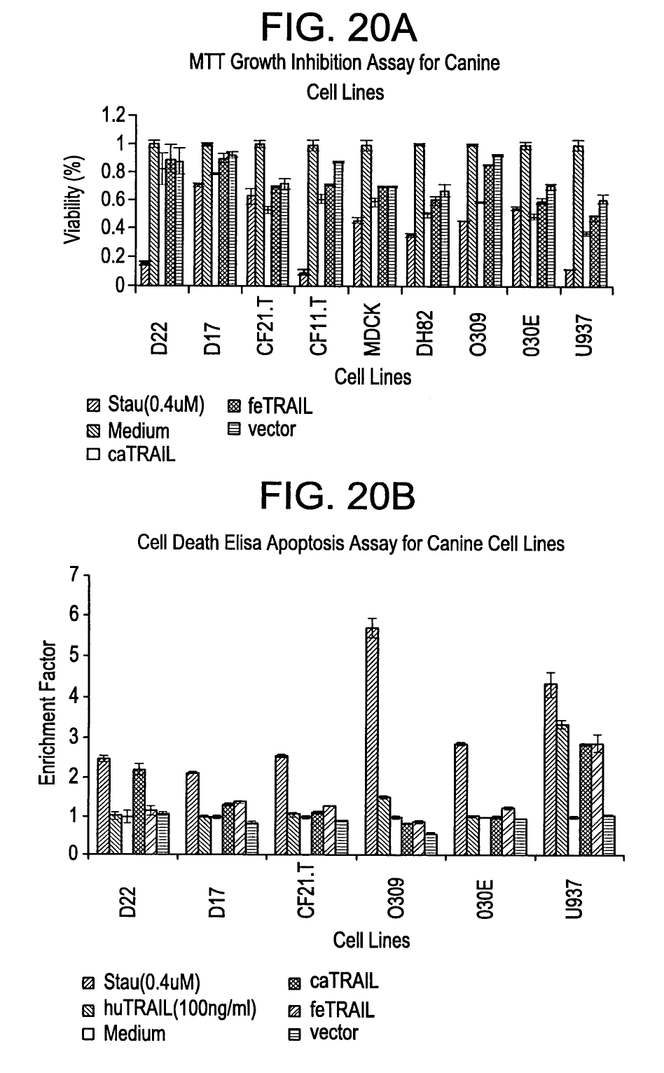

2. Apoptosis-inducing activity of canine and feline TRAIL for various canine tumor cell lines. Eight canine tumor cell lines (D22, canine osteosarcoma; D17, another canine osteosarcoma; CF21.T, canine arm/shoulder cancer; CF11.T, canine osteosarcoma; MDCK, canine kidney cell line; DH82, canine histiocytosis; 0309 and 030E, both independent canine histiocytosis clones) were assayed for their sensitivity against mammalian expressed canine and feline TRAIL proteins. U937 human tumor cell line was assayed at the same time and used as positive control. For MTT cell growth inhibition assay, canine TRAIL containing culture supernatant induced apparent apoptosis for all the cell lines tested; while the effect of feline TRAIL was less clear for cell lines MDCK, CF21.T, D17 and D22 (FIG. 20A). This result demonstrates the efficacy of canine TRAIL as a potent therapeutics for canine tumor cell lines.

For Cell Death ELISA apoptosis assay, canine TRAIL induced apparent apoptosis for D22, D17 and, to a lesser extent, CF21.T and 0309 (FIG. 20B). Feline TRAIL also had an effect on D17, CF21.T, 0309 and 030E. It is interesting to note that although commercial human TRAIL induced potent apoptosis for human U937 cells, its effect on canine cell lines were very minimal (FIG. 20B and data not shown). Canine and feline TRAIL demonstrated potent apoptosis-inducing activity for various human tumor cell lines (FIGS. 19A & 19B). The species specificity of TRAIL for its susceptible cell lines seems to be uni-directional, i.e. canine and feline TRAIL induce apoptosis of human tumor cell lines, while human TRAIL cannot effectively induce apoptosis of canine tumor cell lines.

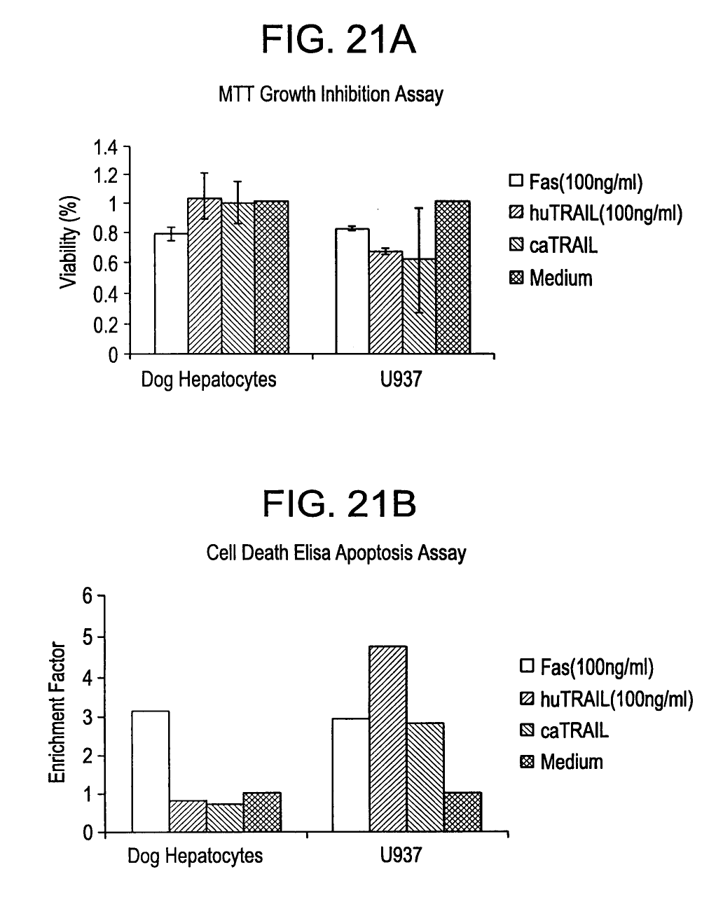

3. Apoptosis-inducing activity of canine and feline TRAIL for normal canine hepatocytes. Normal dog hepatocytes were assayed for their sensitivity against mammalian expressed canine and feline TRAIL proteins. Cell Death ELISA assay clearly demonstrated that while normal dog hepatocytes were very sensitive to Fas ligand treatment, both canine and feline TRAIL proteins had no effect on those cells, while U937 was sensitive to both Fas ligand and TRAIL treatment (FIG. 21B). This conclusion was supported by the result of the MTT assay (FIG. 21A). Fas ligand is a well known agent which causes liver toxicity in vivo. The fact that TRAIL proteins do not cause apoptosis of liver cells in vitro, indicates that there likely to be little, if any, liver toxicity of TRAIL proteins when employed in vivo.

Here the clonings of the canine and feline apoptosis inducer TRAIL genes are reported. The degree of homology between canine TRAIL and that of human and mouse is 80.3% and 63.5% respectively. The degree of homology between feline TRAIL and that of human and mouse is 83.2% and 65.3. From the alignments of all known amino acid sequences of soluble TRAIL, the degree of homology between canine soluble TRAIL and that of human and mouse is 76.9% and 66.9% respectively. The degree of homology between feline soluble TRAIL and that of human and mouse is 82.2% and 69.9% respectively. The degree of homology between soluble canine and feline TRAIL was 92.4%. Western immunoblot assays confirmed that, when TRAIL expressed in mammalian cells, the proteins were secreted into conditioned media. Both canine and feline TRAIL were also shown to specifically induce apoptosis of cancer cells at a level comparable to their human counterpart.

Human TRAIL has been shown to inhibit the growth of a wide variety of primary and metastatic tumors. Furthermore, the treatment with TRAIL can be repeated many times without inducing drug resistance or side effects. These apoptosis inducers can also be conveniently combined with other cancer therapies such as surgery, chemotherapy, radiation therapy and immunotherapy to achieve superior therapeutic effects. These properties have made TRAIL a very attractive candidate for treating canine and feline cancers, where a safe, efficacious, and broad-spectrum therapy is very much in need. However, the successful application of TRAIL as a cancer therapy probably will involve repeated, continuing treatment in order to achieve long-term tumor suppression and dormancy. The cloning and identification of canine and feline TRAIL allows for the treatment of dog and cat tumors using species-specific apoptosis inhibitors, thereby minimizing the risk of evoking immune responses under repeated administration. Finally, spontaneous canine tumors are very similar to their human correlates in histopathologic and biologic behavior (MacEwen, 1990, Cancer Metastasis Rev 9(2): 125-36), therefore experimental results obtained from canine tumors will also provide valuable information for human cancer biology and treatment.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

TABLE 1
Cross-reactivity of canine and feline TRAIL proteins with various anti-human TRAIL antibodies.

| | | | | Working | Degree to | Degree to |
|---|---|---|---|---|---|---|
| Antibodies | Source | Cat. No. | Made in Species | dilution | canine Trail | Feline Trail |
| Anti-human TRAIL | R&D systems | AF375 | Goat | 1:500 | +++ | +++ |
| Anti-human TRAIL | R&D systems | MAB375 | Mouse | 1:250 | + | + |
| Anti-human TRAIL | R&D systems | MAB687 | Mouse | 1:250 | ± | ± |
| Anti-TRAIL IgG | Upstate Biotechnology | 06-954 | Rabbit | 1:500 | − | − |
| Anti-TRAIL | Sigma | T9191 | Rabbit | 1:500 | − | − |

Mammalian expressed canine/feline TRAIL proteins with HA tag were tested with various anti-human TRAIL antibodies in Western immunoblot assays. The intensity was measured at ++++ when anti-HA antibody was used in the assay.

What is claimed:

1. An isolated nucleic acid molecule comprising a nucleotide sequence that encodes a TRAIL comprising:
a) a polypeptide as shown in SEQ ID NO: 21; or
b) a polypeptide as shown in SEQ ID NO: 23.

2. An isolated nucleic acid molecule comprising:
a) a nucleic acid as shown in SEQ ID NO: 20; or
b) a nucleic acid as shown in SEQ ID NO: 22.

3. An isolated nucleic acid molecule comprising a complement of the nucleic acid molecule of any one of claims 1-2.

4. An isolated nucleic acid molecule which hybridizes to the complement of the nucleic acid molecule of any one of claims 1-2 under highly stringent conditions.

5. An isolated nucleic acid molecule which hybridizes to the complement of the nucleic acid molecule of any one of claims 1-2 under moderately stringent conditions.

6. The isolated nucleic acid molecule of claim 4, wherein said isolated nucleic acid molecule encodes a TRAIL, with the proviso that said TRAIL is not human or mouse TRAIL.

7. The isolated nucleic acid molecule of claim 5, wherein said isolated nucleic acid molecule encodes a TRAIL, with the proviso that said TRAIL is not human or mouse TRAIL.

8. A vector comprising the nucleic acid of any one of claims 1-2.

9. An expression vector comprising the nucleic acid of any one of claims 1-2 operatively associated with a regulatory region controlling the expression of the polypeptide encoded by said nucleic acid.

10. A host cell genetically engineered to contain the nucleic acid of any one of claims 1-2.

11. A host cell genetically engineered to express the nucleic acid of any one of claims 1-2 operatively associated with a regulatory region controlling expression of the polypeptide encoded by said nucleic acid.

12. A transgenic, non-human animal which has been genetically engineered to contain a transgene comprising the nucleic acid of any one of claims 1-2.

13. The transgenic, non-human animal of claim 12, wherein the transgene is expressed.

14. An isolated polypeptide comprising an amino acid sequence of:
a) SEQ ID NO: 21; or
b) SEQ ID NO: 23.

15. An antibody which binds to the isolated polypeptide of claim 14.

16. An isolated polypeptide comprising an amino acid sequence encoded by the isolated nucleic acid molecule of claim 4.

17. An isolated polypeptide comprising an amino acid sequence encoded by the isolated nucleic acid molecule of claim 5.

18. An isolated fusion polypeptide comprising a fusion peptide and an amino acid sequence of:
a) SEQ ID NO: 21; or
b) SEQ ID NO: 23.

19. An isolated fusion polypeptide comprising a fusion peptide and an amino acid sequence encoded by the isolated nucleic acid molecule of claim 4.

20. An isolated fusion polypeptide comprising a fusion peptide and an amino acid sequence encoded by the isolated nucleic acid molecule of claim 5.

21. A method for treating an apoptosis-related disorder in a subject comprising administering to the subject a compound which modulates the function, activity and/or expression of a TRAIL-encoding sequence in said subject.

22. The method of claim 21, wherein the compound enhances or increases the function, activity and/or expression of the TRAIL-encoding sequence.

23. The methods of any one of claims 21-22, wherein the compound is selected from the group consisting of a small organic molecule, an antibody, a ribozyme or an antisense molecule.

24. The method of any one of claims 21-22, wherein the apotosis-related disorder is selected from the group consisting of cancer, neurodegenerative disease, lupus erythematosus, rheumatoid arthritis and multiple sclerosis.

25. The method of claim 21, wherein said TRAIL-encoding sequence encodes an amino acid sequence comprising:
a) SEQ ID NO: 21; or
b) SEQ ID NO: 23.

26. The method of claim 21, wherein said subject is a dog or a cat.

27. A method for identifying a compound which modulates expression of a TRAIL-encoding sequence comprising:
a) contacting a test compound to a cell that expresses a TRAIL sequence;
b) measuring a level of TRAIL-encoding sequence expression in the cell;
c) comparing the level of TRAIL-encoding sequence expression in the cell in the presence of the test compound to a level of TRAIL-encoding sequence expression in the cell in the absence of the test compound,
wherein if the level of TRAIL sequence expression in the cell in the presence of the test compound differs from the level of expression of the TRAIL sequence in the cell in the absence of the test compound, a compound that modulates expression of a TRAIL sequence is identified.

28. The method of claim 27, wherein the TRAIL sequence is endogenously expressed within the cell.

29. The method of claim 27, wherein the TRAIL-encoding sequence encodes an amino acid sequence comprising:
a) SEQ ID NO: 21; or
b) SEQ ID NO: 23.

30. The method of claim 27, wherein said TRAIL-encoding sequence comprises:
a) a nucleic acid as set forth in SEQ ID NO: 20; or
b) a nucleic acid as set forth in SEQ ID NO: 22.

31. A method for identifying a compound which modulates activity of an TRAIL sequence product comprising:
a) contacting a test compound to a cell that expresses a TRAIL sequence product;
b) measuring a level of TRAIL sequence product in the cell;
c) comparing the level of TRAIL sequence product activity in the cell in the presence of the test compound to a level of TRAIL sequence product activity in the cell in the absence of the test compound, wherein if the level of TRAIL sequence product activity in the cell in the presence of the test compound differs from the level of TRAIL sequence product activity in the cell in the absence of the test compound, a compound that modulates activity of a TRAIL sequence product is identified.

32. The method of claim 31, wherein the TRAIL sequence product comprises:
a) a peptide as set forth in SEQ ID NO: 21; or
b) a peptide as set forth in SEQ ID NO: 23.

33. A method for modulating the activity and/or expression of a TRAIL-encoding sequence in a cell comprising administering to the cell a compound which modulates the activity and/or expression of a TRAIL-encoding sequence in the cell.

34. The method of claim 33, wherein the compound is selected from the group consisting of a small organic molecule, an antibody, a ribozyme or an antisense molecule.

35. The method of claim 33, wherein said TRAIL-encoding sequence encodes an amino acid sequence comprising:
a) SEQ ID NO: 21; or
b) SEQ ID NO: 23.

36. The method of claim 33, wherein said TRAIL-encoding sequence comprises:
a) a nucleic acid as shown in SEQ ID NO: 20; or
b) a nucleic acid as shown in SEQ ID NO: 22.
